(19) United States
(12) Patent Application Publication
(10) Pub. No.: US 2026/0325663 A1
(43) Pub. Date: Oct. 22, 2026

(54) PHARMACEUTICAL COMPOSITION COMPRISING ANTIBODY-DRUG CONJUGATE

(71) Applicant: CSPC MEGALITH BIOPHARMACEUTICAL CO., LTD., Shijiazhuang (CN)

(72) Inventors: Weiqiang HAN, Shijiazhuang (CN); Ru XIE, Shijiazhuang (CN); Xuming WANG, Shijiazhuang (CN); Li SU, Shijiazhuang (CN); Na ZHANG, Shijiazhuang (CN); Shanshan WANG, Shijiazhuang (CN); Fangyuan TIAN, Shijiazhuang (CN); Xiongfei SUN, Shijiazhuang (CN); Xiwu HUI, Shijiazhuang (CN); Bing YAO, Shijiazhuang (CN)

(21) Appl. No.: 19/484,359

(22) PCT Filed: May 16, 2024
(86) PCT No.: PCT/CN2024/093528

§ 371 Date: Nov. 14, 2025

(30) Foreign Application Priority Data
May 16, 2023 (CN) 202310548571.8

(51) Int. Cl. A61K 47/68; A61K 9/00; A61K 9/19; A61K 47/22; A61K 47/26; A61P 35/00

(52) U.S. Cl. CPC A61K 47/68037; A61K 9/0019; A61K 9/19; A61K 47/22; A61K 47/26; A61K 47/6849; A61K 47/6855; A61K 47/6889; A61P 35/00

(57) ABSTRACT

A pharmaceutical composition comprising an antibody-drug conjugate, a preparation method therefor and a use thereof. Specifically, provided are a pharmaceutical composition comprising an antibody-drug conjugate represented by formula I, which is an injection, a preparation method therefor, and a use thereof in the preparation for a drug for treating proliferative diseases.

### CROSS-REFERENCE TO RELATED APPLICATIONS

The present application is a U.S. National Stage Application under 35 U.S.C. § 371 of International Patent Application No. PCT/CN2024/093528, filed on May 16, 2024, which claims priority to Chinese Application No. 202310548571.8 filed on May 16, 2023, the disclosure of which is incorporated by reference herein in its entirety.

### TECHNICAL FIELD

The present invention relates to a pharmaceutical composition of an antibody-drug conjugate and a process for preparing the composition.

### BACKGROUND OF ART

The antibody-drug conjugate (ADC) consists of three different components (antibody, linker and drug). The ADC technology is to couple an antibody and a drug molecule together through a linker, utilize the specificity of the antibody to transport the drug molecule in a targeted manner to a target tissue to play a role, e.g. reducing the systemic toxic and side effects of the drug, expanding the drug treatment window, and improving the therapeutic potential of the antibody.

EGFR is an expression product of the proto-oncogene c-erbB1, and it is a member of the epidermal growth factor receptor (HER) family. This family includes HER1 (erbB1, EGFR), HER2 (erbB2, NEU), HER3 (erbB3), and HER4 (erbB4). EGFR is a transmembrane protein that plays an important role in cell growth, development, and differentiation. Among others, the overexpression of EGFR and its binding with specific ligands such as EGF and TGFα can lead to abnormal activation of EGFR; the activated EGFR can further activate and regulate multiple signal transduction pathways, thereby inhibiting tumor cell apoptosis, promoting tumor cell proliferation and migration, and promoting tumor angiogenesis by mediating VEGF expression, thus playing an important role in the process such as tumor occurrence, development, malignancy, and prognosis. Therefore, blocking the EGFR-mediated signal transduction pathway can achieve therapeutic effects against tumors.

HER3 (also known as ERBB3, human epidermal growth factor receptor 3) is a transmembrane receptor and a member of the epidermal growth factor receptor family (EGFR). The HER3 gene is located on the long arm of human chromosome 12 (12q13), and it encodes a protein with a molecular weight of either 160 or 180 kDa. HER3 is known to be expressed in various cancers, such as breast cancer, lung cancer, and colorectal cancer, and it forms heterodimers with tyrosine kinase receptors such as HER2 and EGFR, after which HER3 is phosphorylated, thereby inducing cancer cell growth or apoptosis inhibition signals.

CN202211461614.0 discloses an EGFR-targeted antibody-drug conjugate:

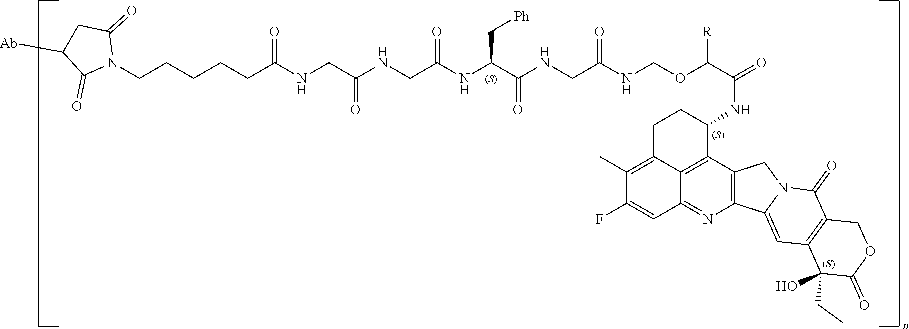

wherein Ab is an antibody targeting HER2, HER3, or EGFR or an antigen-binding fragment thereof, R is selected from C1-6 alkyl, and n is an integer of 1-8 or a decimal of 1-8. In vitro studies have shown that this ADC has good inhibitory effects on various tumor cells.

In the study of antibody and antibody-drug conjugate (ADC) formulations, the formation of aggregates and the generation of degradation products cause pharmaceutically undesirable side effects, which pose risks of increased immunogenicity in patients receiving drug treatment or risks related to venous disorders. For the above reasons, it is necessary to inhibit the formation of aggregates and the generation of degradation products when preparing related formulations. Particularly when studying pharmaceutical formulations of antibody-drug conjugates, more technical challenges may be faced. It is necessary to consider not only the specific properties of the antibody part but also the specific properties of the drug-linker part. For example, during storage, small molecule payloads may detach from the antibody-drug conjugate, which can affect both the efficacy and toxicity of the relevant drug.

It is our research direction to develop stable pharmaceutical compositions to solve the problems of aggregation and degradation of ADC molecules in the pharmaceutical compositions of antibody-drug conjugates.

### SUMMARY OF THE INVENTION

The present invention provides a pharmaceutical composition, which contains an antibody-drug conjugate represented by formula I:

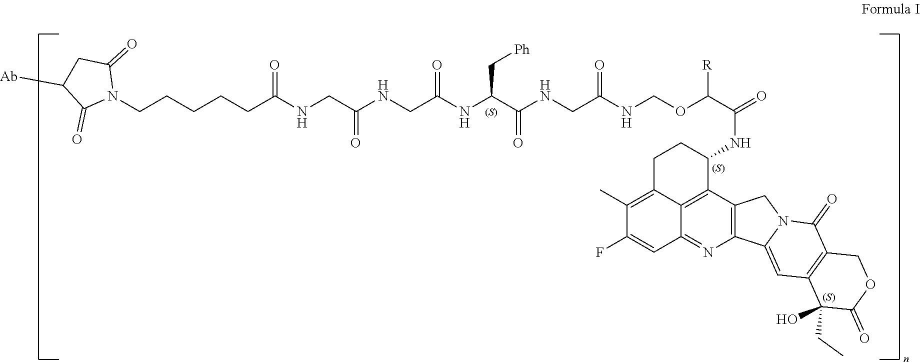

wherein Ab is an antibody targeting HER2, HER3 or EGFR or an antigen-binding fragment thereof, R is C₁₋₆alkyl, and n is an integer of 1-8 or a decimal of 1-8.

Further, n is an integer of 4-8 or a decimal of 4-8.

Further, when n is an integral 1-8, it can be 1, 2, 3, 4, 5, 6, 7, or 8; when n is an integral of 4-8, it can be 4, 5, 6, 7, or 8.

Further, when n is a decimal, it refers to the average number of the linker-drug molecules conjugated per antibody unit.

In a preferable example of the present invention, R is C₁₋₃alkyl.

In a preferable example of the present invention, R is methyl, ethyl, propyl, or isopropyl.

In a preferable example of the present invention, R is methyl.

Further, the antibody-drug conjugate represented by formula I has a structure represented by formula Ia or formula Ib:

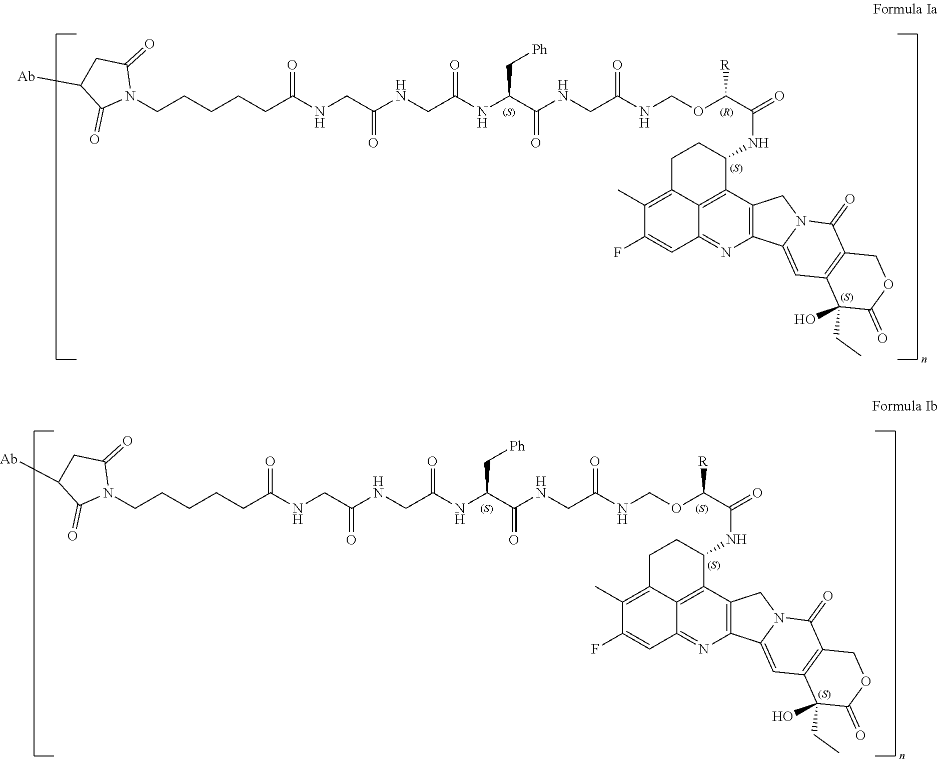

wherein in formula Ia and formula Ib, Ab, R and n are defined as in formula I.

Further, the antibody-drug conjugate represented by formula I has a structure represented by formula I-1, Ia-1 or Ib-1:

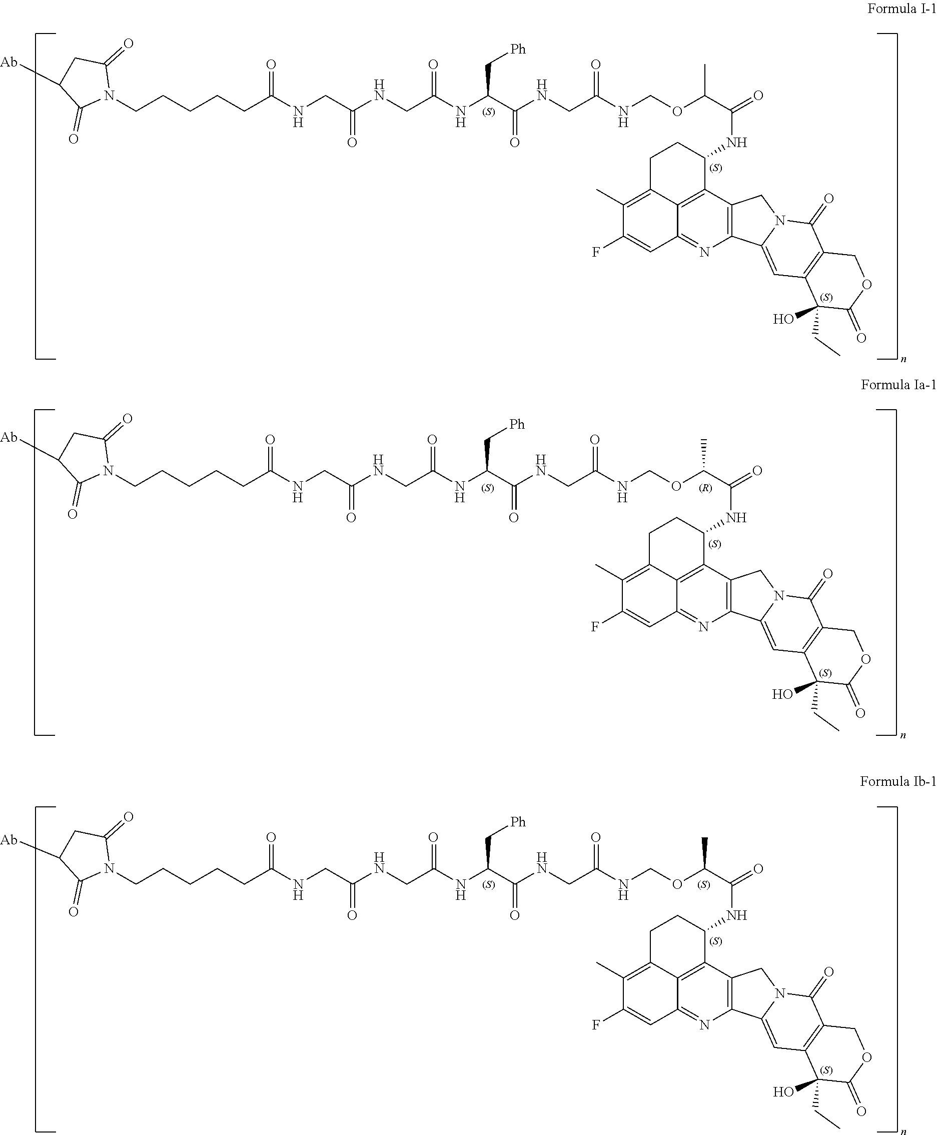

wherein in formula I-1, formula Ia-1 and formula Ib-1, Ab and n are defined as in formula I.

In a preferable example of the present invention, in the above-mentioned antibody-drug conjugate, in case that the Ab is an antibody targeting HER2 or an antigen-binding fragment thereof, it comprises a variable region of a heavy chain (HV) and a variable region of a light chain (LV), wherein, a sequence of the variable region of the heavy chain comprises a heavy chain complementarity determining region 1 (HCDR1), a heavy chain complementarity determining region 2 (HCDR2) and a heavy chain complementarity determining region 3 (HCDR3), a sequence of the variable region of the light chain comprises a light chain complementarity determining region 1 (LCDR1), a light chain complementarity determining region 2 (LCDR2) and a light chain complementarity determining region 3 (LCDR3), wherein the amino acid sequence of HCDR1 is represented by SEQ ID NO:1, the amino acid sequence of HCDR2 is represented by SEQ ID NO:2, the amino acid sequence of HCDR3 is represented by SEQ ID NO:3, and/or the amino acid sequence of LCDR1 is represented by SEQ ID NO:4, the amino acid sequence of LCDR2 is represented by SEQ ID NO:5, the amino acid sequence of LCDR3 is represented by SEQ ID NO:6.

Further preferably, in case that the Ab is an antibody targeting HER2 or an antigen-binding fragment thereof, it comprises a variable region of a heavy chain (HV) and a variable region of a light chain (LV), wherein the amino acid sequence of HV is represented by SEQ ID NO:7, and the amino acid sequence of LV is represented by SEQ ID NO:8.

More preferably, in case that the Ab is an antibody targeting HER2 or an antigen-binding fragment thereof, it comprises a heavy chain (HC) and a light chain (LC), wherein the amino acid sequence of HC is represented by SEQ ID NO:9, and the amino acid sequence of of LC is represented by SEQ ID NO:10.

In a preferable example of the present invention, in case that the Ab is an antibody targeting HER3 or an antigen-binding fragment thereof, it comprises a variable region of a heavy chain (HV) and a variable region of a light chain (LV), wherein, a sequence of the variable region of the heavy chain comprises a heavy chain complementarity determining region 1 (HCDR1), a heavy chain complementarity determining region 2 (HCDR2) and a heavy chain complementarity determining region 3 (HCDR3), a sequence of the variable region of the light chain comprises a light chain complementarity determining region 1 (LCDR1), a light chain complementarity determining region 2 (LCDR2) and a light chain complementarity determining region 3 (LCDR3), wherein the amino acid sequence of HCDR1 is represented by SEQ ID NO:11, the amino acid sequence of HCDR2 is represented by SEQ ID NO:12, the amino acid sequence of HCDR3 is represented by SEQ ID NO:13, and/or the amino acid sequence of LCDR1 is represented by SEQ ID NO:14, the amino acid sequence of LCDR2 is represented by SEQ ID NO:15, the amino acid sequence of LCDR3 is represented by SEQ ID NO:16.

Further preferably, in case that the Ab is an antibody targeting HER3 or an antigen-binding fragment thereof, it comprises a variable region of a heavy chain (HV) and a variable region of a light chain (LV), wherein the amino acid sequence of HV is represented by SEQ ID NO:17, and the amino acid sequence of LV is represented by SEQ ID NO:18.

More preferably, in case that the Ab is an antibody targeting HER3 or an antigen-binding fragment thereof, it comprises a heavy chain (HC) and a light chain (LC), wherein the amino acid sequence of HC is represented by SEQ ID NO:19, the amino acid sequence of LC is represented by SEQ ID NO:20.

In a preferable example of the present invention, in case that the Ab is an antibody targeting EGFR or an antigen-binding fragment thereof, it comprises a variable region of a heavy chain (HV) and a variable region of a light chain (LV), wherein, a sequence of the variable region of the heavy chain comprises a heavy chain complementarity determining region 1 (HCDR1), a heavy chain complementarity determining region 2 (HCDR2) and a heavy chain complementarity determining region 3 (HCDR3), a sequence of the variable region of the light chain comprises a light chain complementarity determining region 1 (LCDR1), a light chain complementarity determining region 2 (LCDR2) and a light chain complementarity determining region 3 (LCDR3), wherein the amino acid sequence of HCDR1 is represented by SEQ ID NO:21, the amino acid sequence of HCDR2 is represented by SEQ ID NO:22, the amino acid sequence of HCDR3 is represented by SEQ ID NO:23, and/or the amino acid sequence of LCDR1 is represented by SEQ ID NO:24, the amino acid sequence of LCDR2 is represented by SEQ ID NO:25, the amino acid sequence of LCDR3 is represented by SEQ ID NO:26.

In a preferable example of the present invention, in case that the Ab is an antibody targeting EGFR or an antigen-binding fragment thereof, it comprises a variable region of a heavy chain (HV) and a variable region of a light chain (LV), wherein the amino acid sequence of HCDR1 is represented by SEQ ID NO:31 or SEQ ID NO:35 or SEQ ID NO:38, the amino acid sequence of HCDR2 is represented by SEQ ID NO:22, the amino acid sequence of HCDR3 is represented by SEQ ID NO:23 or SEQ ID NO:32, and/or the amino acid sequence of LCDR1 is represented by SEQ ID NO:24, the amino acid sequence of LCDR2 is represented by SEQ ID NO:25, the amino acid sequence of LCDR3 is represented by SEQ ID NO:26.

In a preferable example of the present invention, in case that the Ab is an antibody targeting EGFR or an antigen-binding fragment thereof, it comprises a variable region of a heavy chain (HV) and a variable region of a light chain (LV), wherein the amino acid sequence of HCDR1 is represented by SEQ ID NO:31 or SEQ ID NO:35 or SEQ ID NO:38, the amino acid sequence of HCDR2 is represented by SEQ ID NO:22, the amino acid sequence of HCDR3 is represented by SEQ ID NO:32, and the amino acid sequence of LCDR1 is represented by SEQ ID NO:24, the amino acid sequence of LCDR2 is represented by SEQ ID NO:25, the amino acid sequence of LCDR3 is represented by SEQ ID NO:26.

Further preferably, in case that the Ab is an antibody targeting EGFR or an antigen-binding fragment thereof, it comprises a variable region of a heavy chain (HV) and a variable region of a light chain (LV), wherein the amino acid sequence of HV is represented by SEQ ID NO:27, the amino acid sequence of LV is represented by SEQ ID NO:28.

Further preferably, in case that the Ab is an antibody targeting EGFR or an antigen-binding fragment thereof, it comprises a variable region of a heavy chain (HV) and a variable region of a light chain (LV), wherein the amino acid sequence of HV is represented by SEQ ID NO:33 or SEQ ID NO:36 or SEQ ID NO:39, the amino acid sequence of LV is represented by SEQ ID NO:28.

More preferably, in case that the Ab is an antibody targeting EGFR or an antigen-binding fragment thereof, it comprises a heavy chain (HC) and a light chain (LC), wherein the amino acid sequence of HC is represented by SEQ ID NO:29, the amino acid sequence of LC is represented by SEQ ID NO:30.

More preferably, in case that the Ab is an antibody targeting EGFR or an antigen-binding fragment thereof, it comprises a heavy chain (HC) and a light chain (LC), wherein the amino acid sequence of HC is represented by SEQ ID NO:34 or SEQ ID NO:37 or SEQ ID NO:40, the amino acid sequence of LC is represented by SEQ ID NO:30.

In case that Ab is an antibody targeting HER2, HER3 or EGFR, the antibody is preferably a monoclonal antibody, and the monoclonal antibody is preferably selected from human-source antibody, humanized antibody, and chimeric antibody.

In case that Ab is an antigen-binding fragment targeting HER2, HER3 or EGFR, the antigen-binding fragment is preferably selected from Fab′, (Fab′)2, Fab, Fv, scFv, dAb.

In the present invention, the numbering rule of the antibody amino acid sequence adopts the Kabat numbering rule.

Further, the antibody-drug conjugate represented by formula I has a structure as shown below:

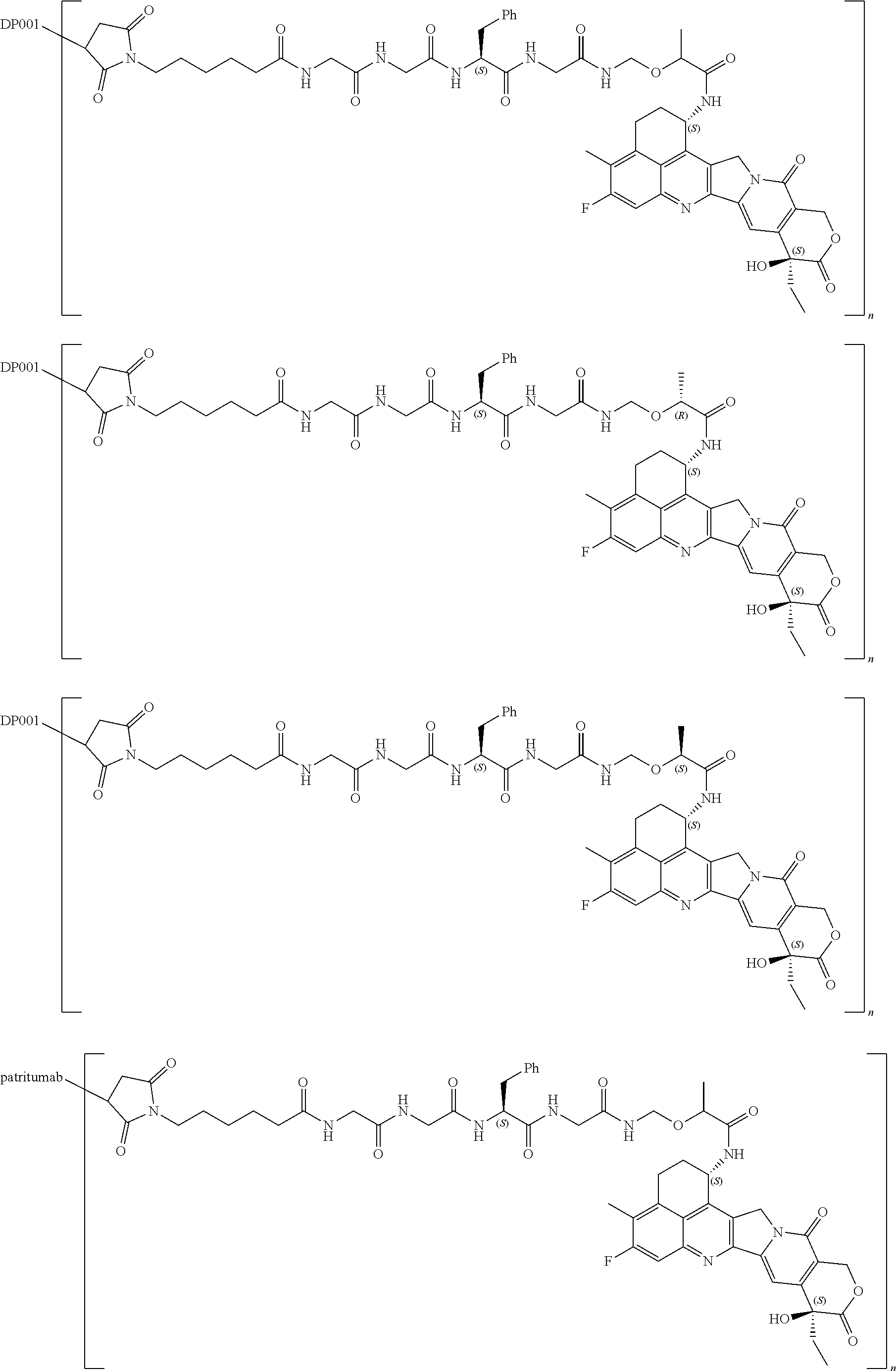

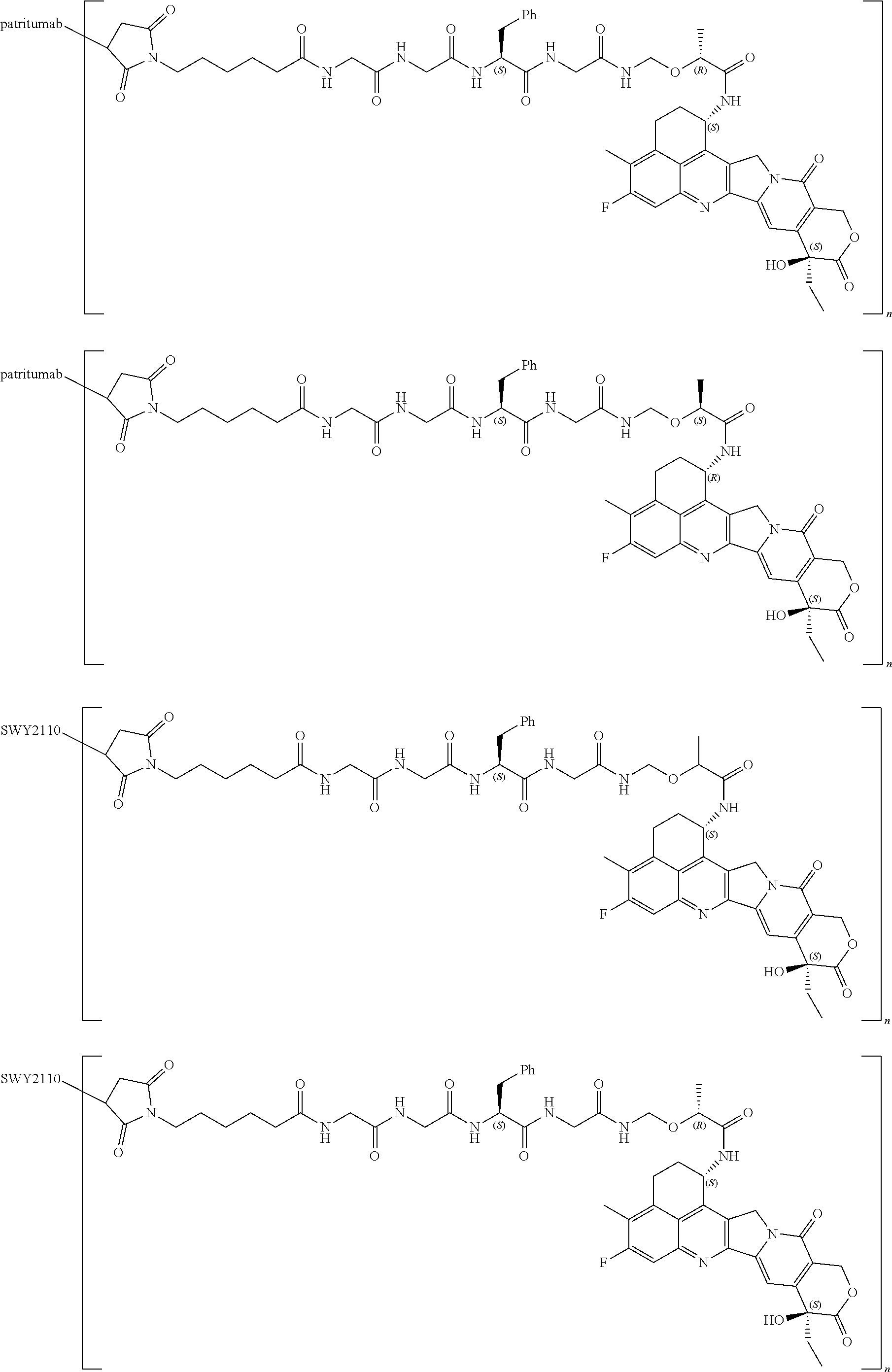

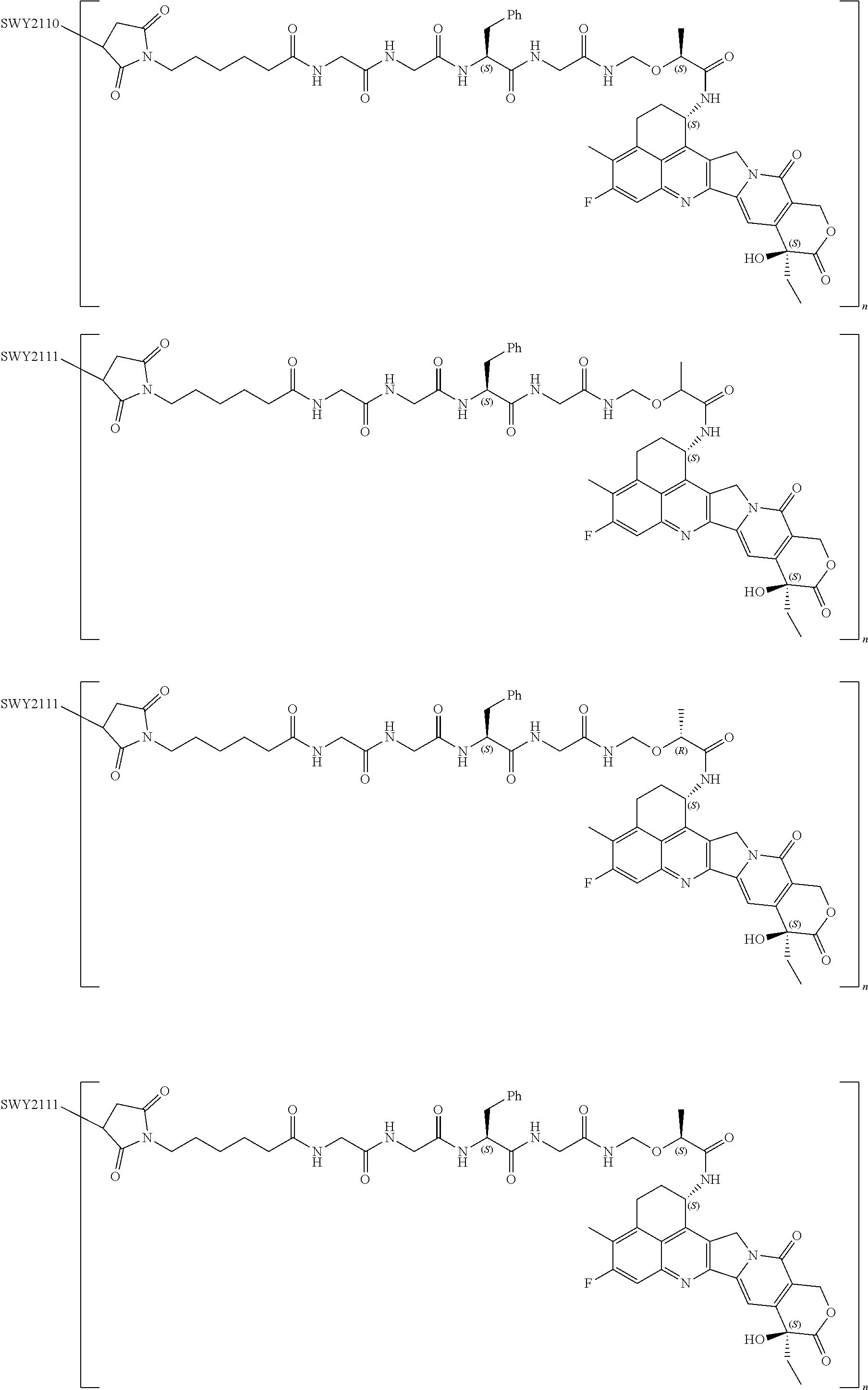

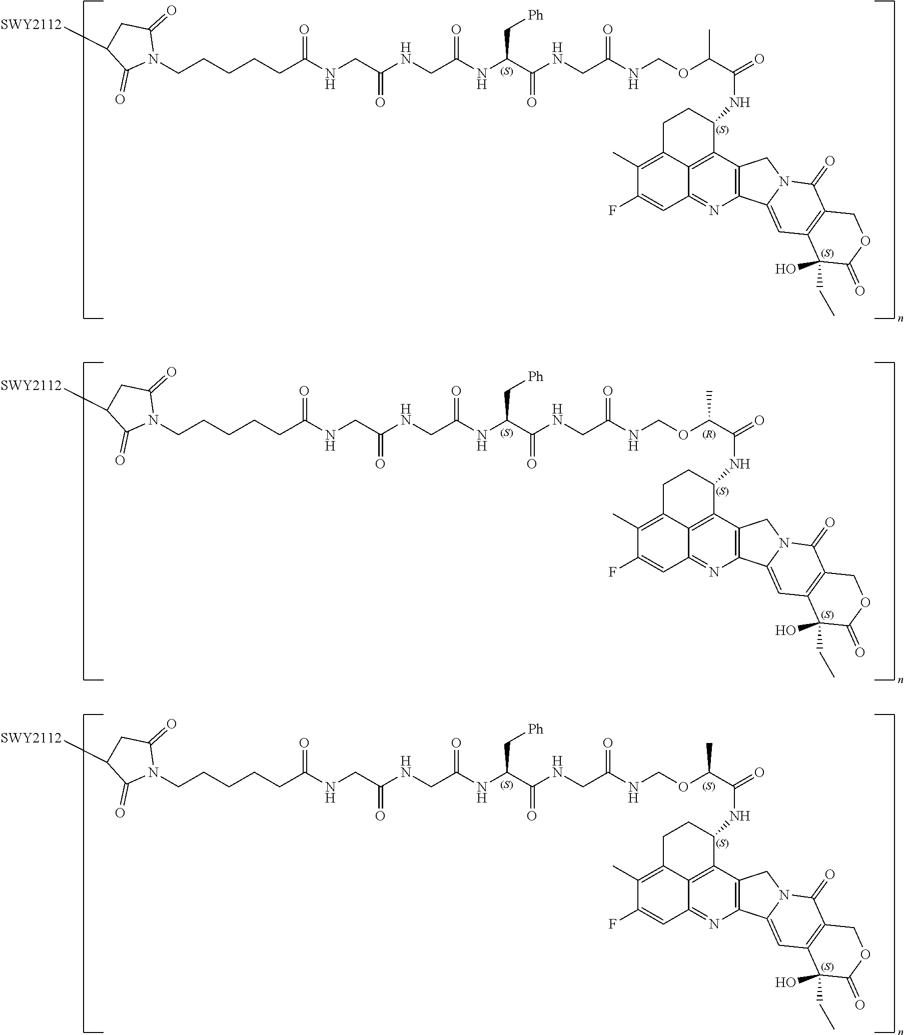

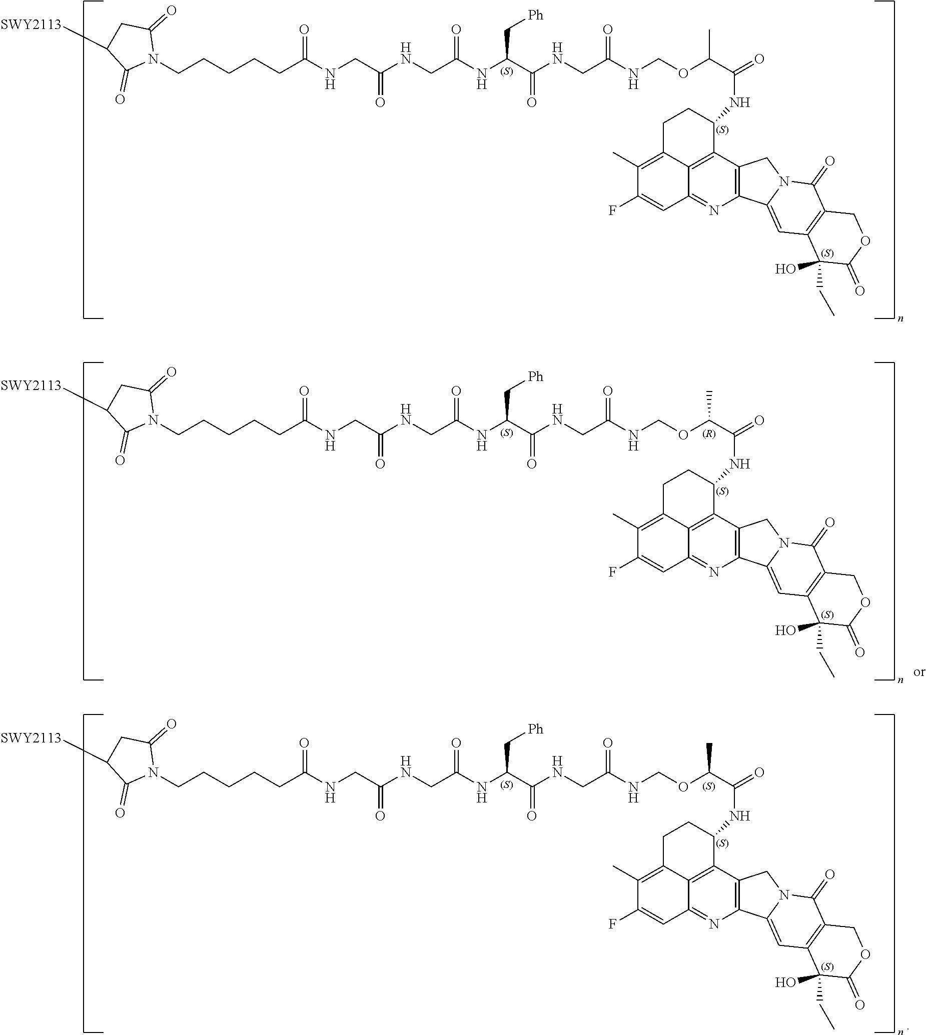

Further, the antibody-drug conjugate represented by formula I has a structure as shown below:

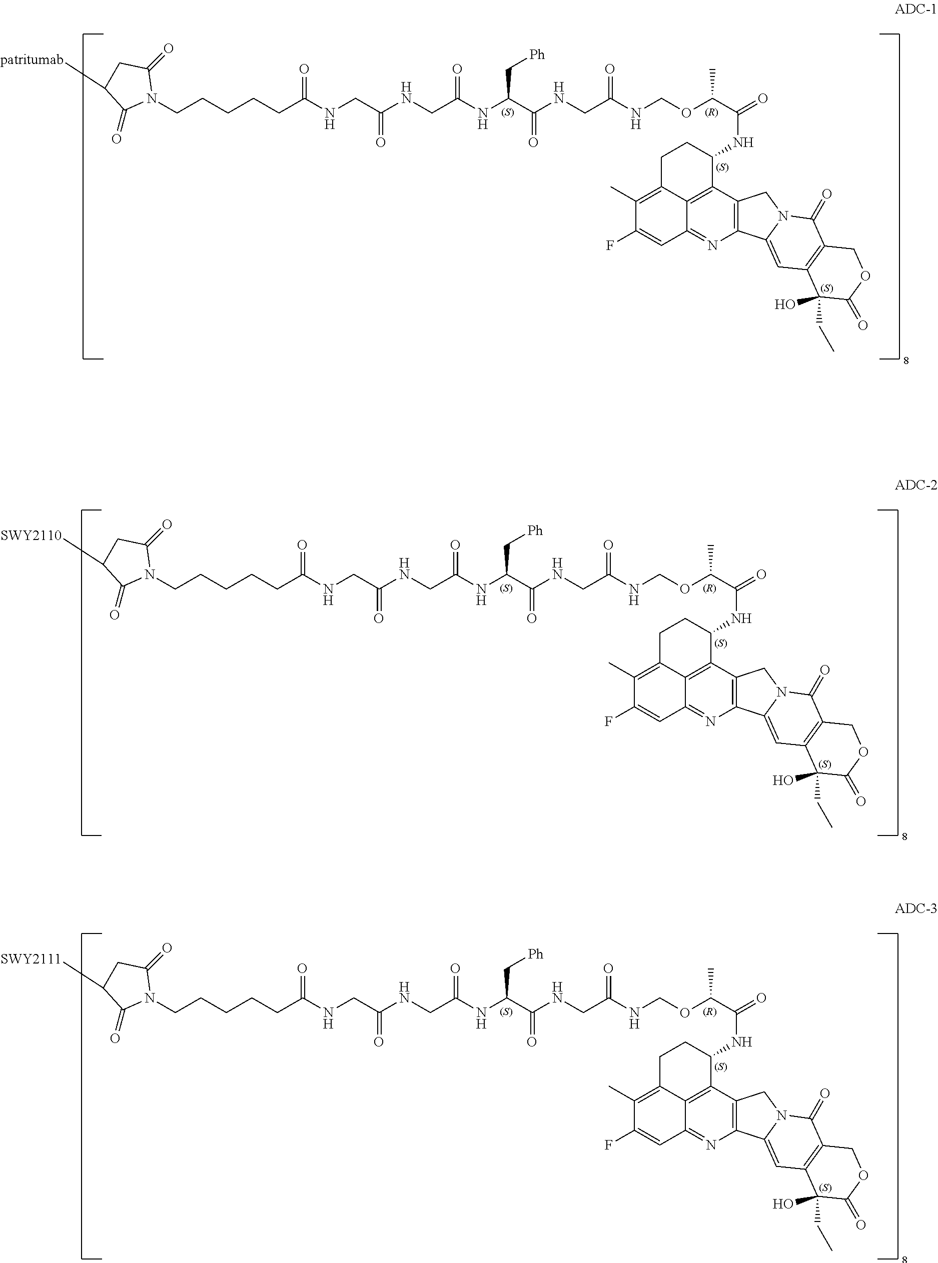

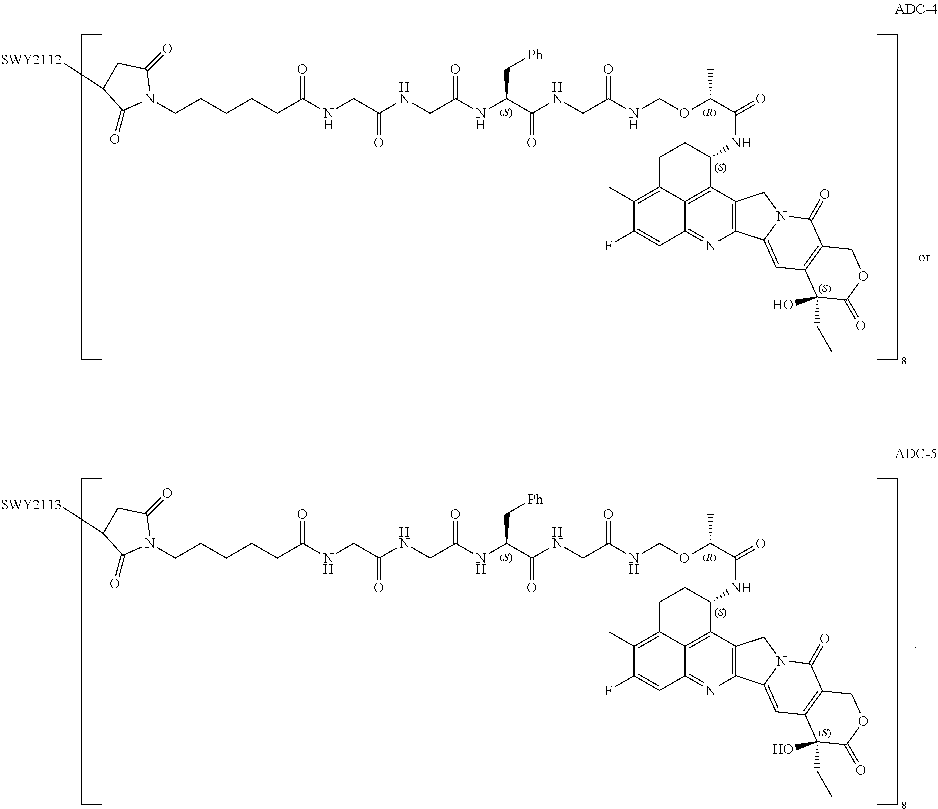

In the above-mentioned antibody-drug conjugates:

DP001 is an antibody having two heavy chains with the amino acid sequence represented by SEQ ID NO:9 and two light chains with the amino acid sequence represented by SEQ ID NO:10;

Patritumab is an antibody having two heavy chains with the amino acid sequence represented by SEQ ID NO:19 and two light chains with the amino acid sequence represented by SEQ ID NO:20;

SWY2110 is an antibody having two heavy chains with the amino acid sequence represented by SEQ ID NO:29 and two light chains with the amino acid sequence represented by SEQ ID NO:30;

SWY2111 is an antibody having two heavy chains with the amino acid sequence represented by SEQ ID NO:34 and two light chains with the amino acid sequence represented by SEQ ID NO:30;

SWY2112 is an antibody having two heavy chains with the amino acid sequence represented by SEQ ID NO:37 and two light chains with the amino acid sequence represented by SEQ ID NO:30;

SWY2113 is an antibody having two heavy chains with the amino acid sequence represented by SEQ ID NO:40 and two light chains with the amino acid sequence represented by SEQ ID NO:30;
n is an integer of 1-8 or a decimal of 1-8.

Further, n is an integer of 4-8 or a decimal of 4-8.

Further, n is 8.

Further, when n is an integral 1-8, it can be 1, 2, 3, 4, 5, 6, 7, or 8; when n is an integral of 4-8, it can be 4, 5, 6, 7, or 8.

Furthermore, the pharmaceutical composition is an injection, preferably an injection solution or a lyophilized injection, more preferably a lyophilized injection.

The lyophilized injection is a solid injection (i.e., lyophilized powder), which can be used by dissolving in water (preferably water for injection) at the time of use, and which is obtained by lyophilizing an aqueous solution containing a predetermined amount of the drug component.

In the present invention, water for injection refers to water that meets the specifications under the section “water for injection” of the Chinese Pharmacopoeia (2020).

Furthermore, the pharmaceutical composition comprises the antibody-drug conjugate represented by formula I and a buffering agent.

Furthermore, the pharmaceutical composition further comprises a stabilizer.

Furthermore, the pharmaceutical composition further comprises a surfactant.

Furthermore, the pharmaceutical composition may further comprise water.

In some embodiments, the pharmaceutical composition comprises the antibody-drug conjugate represented by formula I, a buffering agent, a stabilizer, and a surfactant.

In some embodiments, the pharmaceutical composition further comprises water.

In some embodiments, in the pharmaceutical composition, the antibody-drug conjugate represented by formula I is selected from ADC-1, ADC-2, ADC-3, ADC-4, and ADC-5.

In some embodiments, the buffering agent includes, but is not limited to, acetate, succinate, gluconate, histidine salt, oxalate, lactate, phosphate, citrate, tartrate, fumarate, glycylglycine, tris(hydroxymethyl)aminomethane (Tris), 2-(N-morpholino)ethanesulfonic acid (MES), and other organic acid buffering agents.

In some embodiments, the histidine salt buffering agent is a buffering agent containing histidine ions. Examples of histidine salt buffering agents include histidine-histidine hydrochloride, histidine-acetate, histidine-phosphate, histidine-sulfate, and other buffering agents, wherein the histidine-histidine hydrochloride buffering agent is prepared from histidine and histidine hydrochloride, and the histidine-acetate buffering agent is prepared from histidine and acetic acid. Similarly, the succinate buffering agent may be succinic acid-sodium succinate, and the citrate buffering agent may be citric acid-sodium citrate.

In some embodiments, the histidine-histidine hydrochloride buffering agent is preferred.

In some embodiments, the stabilizer includes, but is not limited to, saccharides (such as sucrose and trehalose), polyols (such as mannitol and sorbitol), amino acids (such as L-serine, sodium glutamate, alanine, glycine, and sarcosine).

In some embodiments, the surfactant includes, but is not limited to, polysorbates (such as polysorbate 20 and polysorbate 80).

In the following embodiments describing pH, weight percentage content (in weight %), and content (in weight/volume such as mg/ml), in the absence of contrary teaching, the pH value of the pharmaceutical composition is the pH value of the aqueous solution of the pharmaceutical composition (wherein the water is water for injection); in the absence of contrary teaching, when describing the weight percentage content, it is based on the total weight of the pharmaceutical composition being 100 wt % and on the premise that the pharmaceutical composition does not include water for injection; in the absence of contrary teaching, when describing the content, the pharmaceutical composition includes water for injection and is based on the volume of the pharmaceutical composition.

In some embodiments, the pH of the pharmaceutical composition is 4.0-7.5 (e.g. about 4.0, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0), preferably 4.5-7.0, further preferably 4.5-5.5, further preferably 5.0-5.5, most preferably about 5.0.

In the above embodiments and similar embodiments below, “about” before a point value includes the value itself and values within an error range understandable by those skilled in the art.

In some embodiments, in the pharmaceutical composition, the weight percentage content of the antibody-drug conjugate represented by formula I (such as ADC-1, ADC-2, ADC-3, ADC-4, and ADC-5) is 1-30% (e.g. about 2%, about 3%, about 4%, about 5%, about 6%, about 7%, about 8%, about 8.5%, about 9%, about 9.5%, about 10%, about 10.5%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%), preferably 5-25%, 8-20%, 8-15%, 8-10%, 15-25%.

In some embodiments, in the pharmaceutical composition, the content of the antibody-drug conjugate represented by formula I (such as ADC-1, ADC-2, ADC-3, ADC-4, and ADC-5) is 1-30 mg/ml (e.g. about 1 mg/ml, about 2 mg/ml, about 3 mg/ml, about 4 mg/ml, about 5 mg/ml, about 6 mg/ml, about 7 mg/ml, about 8 mg/ml, about 8.5 mg/ml, about 9 mg/ml, about 9.5 mg/ml, about 10 mg/ml, about 10.5 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml), preferably 5-25 mg/ml, further preferably 5-20 mg/ml, 5-15 mg/ml, 15-25 mg/ml.

In some embodiments, in the pharmaceutical composition, the weight percentage content of the buffering agent (such as histidine-histidine hydrochloride) is 1-10% (e.g. about 1%, about 2%, about 2.8%, about 2.9%, about 3%, about 3.1%, about 3.2%, about 4%, about 4.5%, about 4.6%, about 4.7%, about 4.8%, about 4.9%, about 5%, about 5.1%, about 5.2%, about 5.3%, about 5.4%, about 5.5%, about 6%, about 6.5%, about 7%, about 8%, about 9%, about 9.9%, about 10%), preferably 1-9.9%, 2-6%, 2-5%, 4-6%.

In some embodiments, in the pharmaceutical composition, the content of the buffering agent (such as histidine-histidine hydrochloride) is 1-10 mg/ml (e.g. about 1 mg/ml, about 2 mg/ml, about 3 mg/ml, about 4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 6 mg/ml, about 6.5 mg/ml, about 7 mg/ml, about 8 mg/ml, about 9 mg/ml, about 10 mg/ml), preferably 2-6 mg/ml, 2-4 mg/ml, 3-6 mg/ml, 4-6 mg/ml.

In some embodiments, in the pharmaceutical composition, the buffering agent is histidine-histidine hydrochloride, wherein the weight percentage content of histidine is 0.1-1% (e.g. about 0.1%, about 0.2%, about 0.25%, about 0.26%, about 0.27%, about 0.28%, about 0.29%, about 0.3%, about 0.31%, about 0.32%, about 0.33%, about 0.34%, about 0.35%, about 0.4%, about 0.45%, about 0.5%, about 0.55%, about 0.6%, about 0.7%, about 0.8%, about 0.9%, about 1%), preferably 0.1-0.5%, 0.2-0.4%.

In some embodiments, in the pharmaceutical composition, the buffering agent is histidine-histidine hydrochloride, wherein the content of histidine is 0.1-1 mg/ml (e.g. about 0.1 mg/ml, about 0.2 mg/ml, about 0.21 mg/ml, about 0.22 mg/ml, about 0.23 mg/ml, about 0.24 mg/ml, about 0.25 mg/ml, about 0.26 mg/ml, about 0.27 mg/ml, about 0.28 mg/ml, about 0.29 mg/ml, about 0.3 mg/ml, about 0.31 mg/ml, about 0.32 mg/ml, about 0.33 mg/ml, about 0.34 mg/ml, about 0.35 mg/ml, about 0.4 mg/ml, about 0.45 mg/ml, about 0.5 mg/ml, about 0.55 mg/ml, about 0.6 mg/ml, about 0.7 mg/ml, about 0.8 mg/ml, about 0.9 mg/ml, about 1 mg/ml), preferably 0.1-0.5 mg/ml, 0.1-0.3 mg/ml, 0.2-0.4 mg/ml.

In some embodiments, in the pharmaceutical composition, the buffering agent is histidine-histidine hydrochloride, wherein the weight percentage content of histidine hydrochloride is 1-10% (e.g. about 1%, about 2%, about 2.5%, about 2.6%, about 2.7%, about 2.8%, about 2.9%, about 3%, about 3.5%, about 4%, about 4.5%, about 4.6%, about 4.7%, about 4.8%, about 4.9%, about 5%, about 5.1%, about 5.2%, about 5.3%, about 5.4%, about 5.5%, about 6%, about 6.5%, about 7%, about 8%, about 9%, about 9.9%, about 10%), preferably 1-9.9%, 2-6%, 2-4%, 3-6%, 3.5-5.5%, 4-5%.

In some embodiments, in the pharmaceutical composition, the buffering agent is histidine-histidine hydrochloride, wherein the content of histidine hydrochloride is 1-10 mg/ml (e.g. about 1 mg/ml, about 2 mg/ml, about 2.5 mg/ml, about 2.6 mg/ml, about 2.7 mg/ml, about 2.8 mg/ml, about 2.9 mg/ml, about 3 mg/ml, about 3.5 mg/ml, about 4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 6 mg/ml, about 6.5 mg/ml, about 7 mg/ml, about 8 mg/ml, about 9 mg/ml, about 10 mg/ml), preferably 2-6 mg/ml, 2-4 mg/ml, 3-6 mg/ml, about 4-5.5 mg/ml, about 4.5-5 mg/ml.

In some embodiments, in the pharmaceutical composition, the weight percentage content of the stabilizer (such as trehalose, sucrose) is 50-90% (e.g. about 50%, about 60%, about 70%, about 75%, about 80%, about 81%, about 82%, about 83%, about 84%, about 85%, about 86%, about 87%, about 88%, about 89%, about 90%), preferably 60-90%, 70-90%, 80-90%.

In some embodiments, in the pharmaceutical composition, the content of the stabilizer (such as trehalose, sucrose) is 50-110 mg/ml (e.g. about 50 mg/ml, about 60 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 100 mg/ml, about 105 mg/ml, about 110 mg/ml), preferably 70-100 mg/ml, 80-100 mg/ml, 75-95 mg/ml.

In some embodiments, in the pharmaceutical composition, the weight percentage content of the surfactant (such as polysorbate 80) is 0.1-1% (e.g. about 0.1%, about 0.15%, about 0.16%, about 0.17%, about 0.18%, about 0.19%, about 0.2%, about 0.25%, about 0.26%, about 0.27%, about 0.28%, about 0.29%, about 0.3%, about 0.31%, about 0.32%, about 0.33%, about 0.34%, about 0.35%, about 0.4%, about 0.45%, about 0.5%, about 0.55%, about 0.6%, about 0.7%, about 0.8%, about 0.9%, about 1%), preferably 0.2-0.4%, further preferably 0.15-0.4%, 0.15-0.35%, 0.25-0.35%.

In some embodiments, in the pharmaceutical composition, the content of the surfactant (such as polysorbate 80) is 0.1-1 mg/ml (e.g. about 0.1 mg/ml, about 0.15 mg/ml, about 0.16 mg/ml, about 0.17 mg/ml, about 0.18 mg/ml, about 0.19 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.7 mg/ml, about 0.8 mg/ml, about 0.9 mg/ml, about lmg/ml), preferably 0.1-0.4 mg/ml, 0.15-0.25 mg/ml, 0.2-0.4 mg/ml, 0.25-0.35 mg/ml.

In some embodiments, the pharmaceutical composition, in terms of weight percentage content, contains 1-30% (e.g. about 2%, about 3%, about 4%, about 5%, about 6%, about 7%, about 8%, about 8.5%, about 9%, about 9.5%, about 10%, about 10.5%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%) of the antibody-drug conjugate represented by formula I (such as ADC-1, ADC-2, ADC-3, ADC-4, and ADC-5), and 0.1-1% (e.g. about 0.1%, about 0.2%, about 0.25%, about 0.26%, about 0.27%, about 0.28%, about 0.29%, about 0.3%, about 0.31%, about 0.32%, about 0.33%, about 0.34%, about 0.35%, about 0.4%, about 0.45%, about 0.5%, about 0.55%, about 0.6%, about 0.7%, about 0.8%, about 0.9%, about 1%) of histidine, 1-10% (e.g. 1-9.9%, or about 1%, about 2%, about 2.5%, about 2.6%, about 2.7%, about 2.8%, about 2.9%, about 3%, about 3.5%, about 4%, about 4.5%, about 4.6%, about 4.7%, about 4.8%, about 4.9%, about 5%, about 5.1%, about 5.2%, about 5.3%, about 5.4%, about 5.5%, about 6%, about 6.5%, about 7%, about 8%, about 9%, about 9.9%, about 10%) of histidine hydrochloride, 50-90% (e.g. about 50%, about 60%, about 70%, about 75%, about 80%, about 81%, about 82%, about 83%, about 84%, about 85%, about 86%, about 87%, about 88%, about 89%, about 90%) of trehalose, 0.1-1% (e.g. about 0.1%, about 0.15%, about 0.16%, about 0.17%, about 0.18%, about 0.19%, about 0.2%, about 0.25%, about 0.26%, about 0.27%, about 0.28%, about 0.29%, about 0.3%, about 0.31%, about 0.32%, about 0.33%, about 0.34%, about 0.35%, about 0.4%, about 0.45%, about 0.5%, about 0.55%, about 0.6%, about 0.7%, about 0.8%, about 0.9%, about 1%) of polysorbate 80.

In some embodiments, the pharmaceutical composition, in terms of weight percentage content, contains 1-30% (e.g. about 2%, about 3%, about 4%, about 5%, about 6%, about 7%, about 8%, about 8.5%, about 9%, about 9.5%, about 10%, about 10.5%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%) of the antibody-drug conjugate represented by formula I (such as ADC-1, ADC-2, ADC-3, ADC-4, and ADC-5), and 0.1-1% (e.g. about 0.1%, about 0.2%, about 0.25%, about 0.26%, about 0.27%, about 0.28%, about 0.29%, about 0.3%, about 0.31%, about 0.32%, about 0.33%, about 0.34%, about 0.35%, about 0.4%, about 0.45%, about 0.5%, about 0.55%, about 0.6%, about 0.7%, about 0.8%, about 0.9%, about 1%) of histidine, 1-10% (e.g. 1-9.9%, or about 1%, about 2%, about 2.5%, about 2.6%, about 2.7%, about 2.8%, about 2.9%, about 3%, about 3.5%, about 4%, about 4.5%, about 4.6%, about 4.7%, about 4.8%, about 4.9%, about 5%, about 5.1%, about 5.2%, about 5.3%, about 5.4%, about 5.5%, about 6%, about 6.5%, about 7%, about 8%, about 9%, about 9.9%, about 10%) of histidine hydrochloride, 50-90% (e.g. about 50%, about 60%, about 70%, about 75%, about 80%, about 81%, about 82%, about 83%, about 84%, about 85%, about 86%, about 87%, about 88%, about 89%, about 90%) of sucrose, 0.1-1% (e.g. about 0.1%, about 0.15%, about 0.16%, about 0.17%, about 0.18%, about 0.19%, about 0.2%, about 0.25%, about 0.26%, about 0.27%, about 0.28%, about 0.29%, about 0.3%, about 0.31%, about 0.32%, about 0.33%, about 0.34%, about 0.35%, about 0.4%, about 0.45%, about 0.5%, about 0.55%, about 0.6%, about 0.7%, about 0.8%, about 0.9%, about 1%) of polysorbate 80.

In some embodiments, the pharmaceutical composition contains 1-30 mg/ml (e.g. about 1 mg/ml, about 2 mg/ml, about 3 mg/ml, about 4 mg/ml, about 5 mg/ml, about 6 mg/ml, about 7 mg/ml, about 8 mg/ml, about 8.5 mg/ml, about 9 mg/ml, about 9.5 mg/ml, about 10 mg/ml, about 10.5 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml) of the antibody-drug conjugate represented by formula I (such as ADC-1, ADC-2, ADC-3, ADC-4, and ADC-5), and 0.1-1 mg/ml (e.g. about 0.1 mg/ml, about 0.2 mg/ml, about 0.21 mg/ml, about 0.22 mg/ml, about 0.23 mg/ml, about 0.24 mg/ml, about 0.25 mg/ml, about 0.26 mg/ml, about 0.27 mg/ml, about 0.28 mg/ml, about 0.29 mg/ml, about 0.3 mg/ml, about 0.31 mg/ml, about 0.32 mg/ml, about 0.33 mg/ml, about 0.34 mg/ml, about 0.35 mg/ml, about 0.4 mg/ml, about 0.45 mg/ml, about 0.5 mg/ml, about 0.55 mg/ml, about 0.6 mg/ml, about 0.7 mg/ml, about 0.8 mg/ml, about 0.9 mg/ml, about lmg/ml) of histidine, 1-10 mg/ml (e.g. about 1 mg/ml, about 2 mg/ml, about 2.5 mg/ml, about 2.6 mg/ml, about 2.7 mg/ml, about 2.8 mg/ml, about 2.9 mg/ml, about 3 mg/ml, about 3.5 mg/ml, about 4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 6 mg/ml, about 6.5 mg/ml, about 7 mg/ml, about 8 mg/ml, about 9 mg/ml, about 10 mg/ml) of histidine hydrochloride, 50-110 mg/ml (e.g. about 50 mg/ml, about 60 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 100 mg/ml, about 105 mg/ml, about 110 mg/ml) of trehalose, 0.1-1 mg/ml (e.g. about 0.1 mg/ml, about 0.15 mg/ml, about 0.16 mg/ml, about 0.17 mg/ml, about 0.18 mg/ml, about 0.19 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.7 mg/ml, about 0.8 mg/ml, about 0.9 mg/ml, about 1 mg/ml) of polysorbate 80.

In some embodiments, the pharmaceutical composition contains 1-30 mg/ml (e.g. about 1 mg/ml, about 2 mg/ml, about 3 mg/ml, about 4 mg/ml, about 5 mg/ml, about 6 mg/ml, about 7 mg/ml, about 8 mg/ml, about 8.5 mg/ml, about 9 mg/ml, about 9.5 mg/ml, about 10 mg/ml, about 10.5 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml) of the antibody-drug conjugate represented by formula I (e.g. ADC-1, ADC-2, ADC-3, ADC-4, ADC-5), and 0.1-1 mg/ml (e.g. about 0.1 mg/ml, about 0.2 mg/ml, about 0.21 mg/ml, about 0.22 mg/ml, about 0.23 mg/ml, about 0.24 mg/ml, about 0.25 mg/ml, about 0.26 mg/ml, about 0.27 mg/ml, about 0.28 mg/ml, about 0.29 mg/ml, about 0.3 mg/ml, about 0.31 mg/ml, about 0.32 mg/ml, about 0.33 mg/ml, about 0.34 mg/ml, about 0.35 mg/ml, about 0.4 mg/ml, about 0.45 mg/ml, about 0.5 mg/ml, about 0.55 mg/ml, about 0.6 mg/ml, about 0.7 mg/ml, about 0.8 mg/ml, about 0.9 mg/ml, about 1 mg/ml) of histidine, 1-10 mg/ml (e.g. about 1 mg/ml, about 2 mg/ml, about 2.5 mg/ml, about 2.6 mg/ml, about 2.7 mg/ml, about 2.8 mg/ml, about 2.9 mg/ml, about 3 mg/ml, about 3.5 mg/ml, about 4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 6 mg/ml, about 6.5 mg/ml, about 7 mg/ml, about 8 mg/ml, about 9 mg/ml, about 10 mg/ml) of histidine hydrochloride, 50-110 mg/ml (e.g. about 50 mg/ml, about 60 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 100 mg/ml, about 105 mg/ml, about 110 mg/ml) of sucrose, 0.1-1 mg/ml (e.g. about 0.1 mg/ml, about 0.15 mg/ml, about 0.16 mg/ml, about 0.17 mg/ml, about 0.18 mg/ml, about 0.19 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.7 mg/ml, about 0.8 mg/ml, about 0.9 mg/ml, about 1 mg/ml) of polysorbate 80.

In some embodiments, after the pharmaceutical composition is dissolved, it contains about 10 mg/ml of the antibody-drug conjugate represented by formula I, about 0.33 mg/ml of histidine, about 4.79 mg/ml of histidine hydrochloride, about 0.3 mg/ml of polysorbate 80, about 90 mg/ml of trehalose, and has a pH of about 5.0.

In some embodiments, the pharmaceutical composition contains about 10 mg/ml of the antibody-drug conjugate represented by formula I, about 0.33 mg/ml of histidine, about 4.79 mg/ml of histidine hydrochloride, about 0.3 mg/ml of polysorbate 80, about 90 mg/ml of trehalose, and has a pH of about 5.0.

In some embodiments, the pharmaceutical composition is a lyophilized injection, and when the lyophilized injection is diluted with water for injection, the diluted pharmaceutical composition contains about 10 mg/ml of the antibody-drug conjugate represented by formula I, about 0.33 mg/ml of histidine, about 4.79 mg/ml of histidine hydrochloride, about 0.3 mg/ml of polysorbate 80, about 90 mg/ml of trehalose, based on the total volume of the diluted pharmaceutical composition, and the pH of the diluted pharmaceutical composition is about 5.0, preferably, the antibody-drug conjugate represented by formula I is selected from SWY2110-JSSW-001, SWY2111-JSSW-001, and SWY2113-JSSW-001, preferably SWY2110-JSSW-001.

In some embodiments, the pharmaceutical composition is an injection solution, and the injection solution contains about 10 mg/ml of the antibody-drug conjugate represented by formula I, about 0.33 mg/ml of histidine, about 4.79 mg/ml of histidine hydrochloride, about 0.3 mg/ml of polysorbate 80, and about 90 mg/ml of trehalose, and the pH of the injection solution is about 5.0, preferably, the antibody-drug conjugate represented by formula I is selected from SWY2110-JSSW-001, SWY2111-JSSW-001, and SWY2113-JSSW-001, preferably SWY2110-JSSW-001.

In other words, in an embodiment of the present invention, the pharmaceutical composition contains about 10 mg/ml of the antibody-drug conjugate represented by formula I, about 0.33 mg/ml of histidine, about 4.79 mg/ml of histidine hydrochloride, about 0.3 mg/ml of polysorbate 80, about 90 mg/ml trehalose, and the balance of water, and the pH of the pharmaceutical composition is about 5.0, preferably, the antibody-drug conjugate represented by formula I is selected from SWY2110-JSSW-001, SWY2111-JSSW-001, and SWY2113-JSSW-001, preferably SWY2110-JSSW-001; or the pharmaceutical composition contains or consists of about 10 parts by weight of the antibody-drug conjugate represented by formula I, about 0.33 parts by weight of histidine, about 4.79 parts by weight of histidine hydrochloride, about 0.3 parts by weight of polysorbate 80, about 90 parts by weight of trehalose, preferably, the antibody-drug conjugate represented by formula I is selected from SWY2110-JSSW-001, SWY2111-JSSW-001, and SWY2113-JSSW-001, preferably SWY2110-JSSW-001.

In some embodiments, after the pharmaceutical composition is dissolved, it contains about 20 mg/ml of the antibody-drug conjugate represented by formula I, about 0.21 mg/ml of histidine, about 2.86 mg/ml of histidine hydrochloride, about 0.2 mg/ml of polysorbate 80, and about 80 mg/ml of sucrose, and has a pH of about 5.0.

In some embodiments, the pharmaceutical composition contains about 20 mg/ml of the antibody-drug conjugate represented by formula I, about 0.21 mg/ml of histidine, about 2.86 mg/ml of histidine hydrochloride, about 0.2 mg/ml of polysorbate 80, and about 80 mg/ml of sucrose, and has a pH of 5.0.

In some embodiments, the pharmaceutical composition is a lyophilized injection, and when the lyophilized injection is diluted with water for injection, the diluted pharmaceutical composition contains about 20 mg/ml of the antibody-drug conjugate represented by formula I, about 0.21 mg/ml of histidine, about 2.86 mg/ml of histidine hydrochloride, about 0.2 mg/ml of polysorbate 80, and about 80 mg/ml of sucrose, based on the total volume of the diluted pharmaceutical composition, and the pH of the diluted pharmaceutical composition is about 5.0, preferably, the antibody-drug conjugate represented by formula I is patritumab-JSSW-001.

In some embodiments, the pharmaceutical composition is an injection solution, and the injection solution contains about 20 mg/ml of the antibody-drug conjugate represented by formula I, about 0.21 mg/ml of histidine, about 2.86 mg/ml of histidine hydrochloride, about 0.2 mg/ml of polysorbate 80, and about 80 mg/ml of sucroseAnd the pH of the injection solution is about 5.0, preferably, the antibody-drug conjugate represented by formula I is patritumab-JSSW-001.

In other words, in an embodiment of the present invention, the pharmaceutical composition contains about 20 mg/ml of the antibody-drug conjugate represented by formula I, about 0.21 mg/ml of histidine, about 2.86 mg/ml of histidine hydrochloride, about 0.2 mg/ml of polysorbate 80, about 80 mg/ml of sucrose, and the balance of water, and the pH of the pharmaceutical composition is about 5.0, preferably, the antibody-drug conjugate represented by formula I is patritumab-JSSW-001; or the pharmaceutical composition contains or consists of about 20 parts by weight of the antibody-drug conjugate represented by formula I, about 0.21 parts by weight of histidine, about 2.86 parts by weight of histidine hydrochloride, about 0.2 parts by weight of polysorbate 80, and about 80 parts by weight of sucrose, preferably, the antibody-drug conjugate represented by formula I is patritumab-JSSW-001.

In some embodiments, the antibody-drug conjugate represented by formula I is one of ADC-1, ADC-2, ADC-3, ADC-4, and ADC-5.

In another aspect, the present invention also relates to a method for preparing the pharmaceutical composition, wherein the method comprises the following steps:
S1) preparing the antibody-drug conjugate represented by formula I;
S2) preparing an ultrafiltration-exchanging buffer;
S3) exchanging the antibody-drug conjugate obtained in S1 into the buffer obtained in S2.

Optionally, the method further comprises S4) freeze-drying.

Furthermore, step S2) comprises weighing a buffering agent and a stabilizer according to the amounts in a formula, adding water for injection to dilute to the target formulation volume, stirring and mixing uniformly to obtain the exchanging buffer.

Furthermore, step S3) comprises exchanging a certain amount of the antibody-drug conjugate obtained in S1 into the buffer obtained in S2 through ultrafiltration with an ultrafiltration membrane, diluting the antibody-drug conjugate to the target concentration, and then adding the surfactant according to its volume fraction to obtain a stock solution.

Furthermore, in step S3, the preparation method optionally comprises a step of sterile filtration of the obtained buffer.

The present invention provides use of the pharmaceutical composition mentioned in the present invention in manufacture of a medicament for treating proliferative diseases. The proliferative disease is preferably a disease associated with abnormal expression of HER2, HER3 or EGFR, including cancer. The cancer is selected from breast cancer, ovarian cancer, cervical cancer, uterine cancer, prostate cancer, kidney cancer, urethra cancer, bladder cancer, liver cancer, gastric cancer, endometrial cancer, salivary gland cancer, esophageal cancer, melanoma, neuroglioma, neuroblastoma, sarcoma, lung cancer (e.g. small cell lung cancer and non-small cell lung cancer), colon cancer, rectal cancer, colorectal cancer, leukemia (e.g. acute lymphoblastic leukemia, acute myeloid leukemia, acute promyelocytic leukemia, chronic myelocytic leukemia, chronic lymphocytic leukemia), bone cancer, skin cancer, thyroid cancer, pancreatic cancer or lymphoma (e.g. Hodgkin lymphoma, non-Hodgkin lymphoma or relapsed anaplastic large cell lymphoma).

The present invention provides a method for treating or preventing proliferative diseases, which method includes administering a therapeutically effective dose of the pharmaceutical composition of the present invention to a patient in need thereof; the proliferative disease is preferably a disease associated with abnormal expression of HER2, HER3 or EGFR, including cancer. The cancer is selected from breast cancer, ovarian cancer, cervical cancer, uterine cancer, prostate cancer, kidney cancer, urethra cancer, bladder cancer, liver cancer, gastric cancer, endometrial cancer, salivary gland cancer, esophageal cancer, melanoma, neuroglioma, neuroblastoma, sarcoma, lung cancer (e.g. small cell lung cancer and non-small cell lung cancer), colon cancer, rectal cancer, colorectal cancer, leukemia (e.g. acute lymphoblastic leukemia, acute myeloid leukemia, acute promyelocytic leukemia, chronic myelocytic leukemia, chronic lymphocytic leukemia), bone cancer, skin cancer, thyroid cancer, pancreatic cancer or lymphoma (e.g. Hodgkin lymphoma, non-Hodgkin lymphoma or relapsed anaplastic large cell lymphoma).

The present invention provides use of the pharmaceutical composition mentioned in the present invention in manufacture of a medicament for treating a drug-resistant proliferative disease. The drug-resistant proliferative disease is a drug-resistant disease associated with abnormal expression of HER2, HER3 or EGFR, including cancer. The drug-resistant cancer is preferably a drug-resistant cancer caused by mutation in HER2, HER3 or EGFR genes. The cancer is preferably breast cancer, ovarian cancer, cervical cancer, uterine cancer, prostate cancer, kidney cancer, urethra cancer, bladder cancer, liver cancer, gastric cancer, endometrial cancer, salivary gland cancer, esophageal cancer, melanoma, neuroglioma, neuroblastoma, sarcoma, lung cancer (e.g. small cell lung cancer and non-small cell lung cancer), colon cancer, rectal cancer, colorectal cancer, leukemia (e.g. acute lymphoblastic leukemia, acute myeloid leukemia, acute promyelocytic leukemia, chronic myelocytic leukemia, chronic lymphocytic leukemia), bone cancer, skin cancer, thyroid cancer, pancreatic cancer or lymphoma (e.g. Hodgkin lymphoma, non-Hodgkin lymphoma or relapsed anaplastic large cell lymphoma), further preferably colon cancer, rectal cancer, lung cancer or pancreatic cancer. Among others, the drug resistance preferably refers to being resistant to receptor tyrosine kinase inhibitors, further preferably resistant to first-generation, second-generation or third-generation EGFR inhibitors, still further preferably resistant to gefitinib, erlotinib, icotinib, afatinib, dacomitinib, osimertinib or almonertinib, especially resistant to gefitinib, afatinib, osimertinib or almonertinib, more preferably resistant to gefitinib or osimertinib.

The present invention provides a method for treating or preventing a drug-resistant proliferative disease, which method includes administering a therapeutically effective dose of the pharmaceutical composition of the present invention to a patient in need thereof; the drug-resistant proliferative disease is a drug-resistant disease associated with abnormal expression of HER2, HER3 or EGFR, including cancer. The drug-resistant cancer is preferably a drug-resistant cancer caused by mutation in HER2, HER3 or EGFR genes. The cancer is preferably breast cancer, ovarian cancer, cervical cancer, uterine cancer, prostate cancer, kidney cancer, urethra cancer, bladder cancer, liver cancer, gastric cancer, endometrial cancer, salivary gland cancer, esophageal cancer, melanoma, neuroglioma, neuroblastoma, sarcoma, lung cancer (e.g. small cell lung cancer and non-small cell lung cancer), colon cancer, rectal cancer, colorectal cancer, leukemia (e.g. acute lymphoblastic leukemia, acute myeloid leukemia, acute promyelocytic leukemia, chronic myelocytic leukemia, chronic lymphocytic leukemia), bone cancer, skin cancer, thyroid cancer, pancreatic cancer or lymphoma (e.g. Hodgkin lymphoma, non-Hodgkin lymphoma or relapsed anaplastic large cell lymphoma), further preferably colon cancer, rectal cancer, lung cancer or pancreatic cancer. Among others, the drug resistance preferably refers to being resistant to receptor tyrosine kinase inhibitors, further preferably resistant to first-generation, second-generation or third-generation EGFR inhibitors, still further preferably resistant to gefitinib, erlotinib, icotinib, afatinib, dacomitinib, osimertinib or almonertinib, especially resistant to gefitinib, afatinib, osimertinib or almonertinib, more preferably resistant to gefitinib or osimertinib.

The pharmaceutical composition of the present invention can be used alone or in combination with other anti-tumor agents.

The process for preparing the pharmaceutical composition mentioned in the present invention comprises the following steps:
S1. Reduction of antibody;

Specifically, the antibody medium is exchanged with PBS6.0/EDTA to prepare a solution having an antibody concentration of 10 mg/mL. An appropriate amount of 10 mM aqueous TCEP solution and 1M aqueous dipotassium hydrogen phosphate solution are added according to the reduction amount. After confirming that the pH of the solution is proper, the incubation at 37° C. is performed for several hours to reduce the disulfide bonds in the antibody.
S2: Conjugation of antibody and linker-drug compound;

Specifically an appropriate amount of a solution (10 mM) of the compound dissolved in dimethyl sulfoxide is added to the above solution at room temperature, and the mixture is mixed evenly and reacted at room temperature for 0.5-4 hours to attach the linker-drug compound to the antibody. Next, 100 mM aqueous NAC solution is added, and the mixture is further stirred at room temperature to terminate the reaction.
S3. Purification of antibody-drug conjugates:

Specifically, the reaction solution is purified by ultrafiltration using ultrafiltration centrifuge tubes. 25 mM 2-(N-morpholine)ethanesulfonic acid (MES) is added as purification buffer to the reaction solution, pH=6.5. The resulting solution is concentrated to 1-2 mL, the purification buffer is added again to make the exchanging factor greater than 1000 times. Thus, the non-conjugated linker-drug compound and other low molecular weight reagents are removed to obtain the purified antibody-drug conjugate.

Compared with known antibody-drug conjugates in the prior art such as DS-8201 or current laboratory samples with better effects on different targets as controls, the antibody-drug conjugates contained in the pharmaceutical composition of the present invention show better anti-cell proliferation effect and significant tumor inhibitory effect. For drug-resistant tumors such as those caused by mutation in HER2, HER3 or EGFR genes, especially those resistant to anti-tumor agents of receptor tyrosine kinase inhibitors, the antibody-drug conjugates of the present invention have superior inhibitory activity to DS-8201, have better stability, including plasma stability and buffer stability, have lower toxicity, have better bystander effect, and have a wider therapeutic window. The present invention carries out the pH-dependent modification of the antibody, and the ADC obtained by conjugating the modified antibody with the linker-drug compound has comparable tumor inhibitory activity to the original ADC in terms of efficacy, and it shows reduced on-target toxicity to normal tissues in terms of safety. The ADC obtained after antibody optimization further expands the therapeutic window.

The pharmaceutical composition of the present invention can be stored for an extended period at 2-8° C. under a specified condition, ensuring the stability of the physicochemical properties of the antibody component while minimizing the dissociation of free drugs. Long-term stability studies showed no significant changes in the physicochemical properties of the antibody component, meeting the storage requirements for antibody formulations. Additionally, the drug-to-antibody ratio (DAR) and the distribution of DAR8 in the ADC portion exhibited no significant changes, indicating consistent quality of the conjugated drug during storage. Although some dissociation of free drugs occurred, the amount was minimal and did not pose any safety risks for clinical administration, thereby allowing for long-term storage.

### Definition

Unless otherwise specified, the term “antibody-drug conjugate (ADC)” refers to the linkage of an antibody (such as a monoclonal antibody) or an antibody fragment to a biologically active cytotoxin drug through a stable chemical linker compound.

Unless otherwise specified, the term “linker-drug compound” refers to a partial structure in the “antibody-drug conjugate”, which is composed of the linker compound and the drug compound.

In the present invention, the linker-drug compound and the antibody are attached by conjugation manners conventionally in the art, including lysine conjugation, reductive disulfide bond conjugation between light and heavy chains, and directional conjugation (Beck, Alain, and Janice M. Reichert. “Antibody-drug conjugates: present and future.” MAbs. Vol. 6. No. 1. Taylor & Francis, 2014.; McCombs, Jessica R., and Shawn C. Owen. “Antibody drug conjugates: design and selection of linker, payload and conjugation chemistry.” The AAPS journal 17 (2015): 339-351.). In the present invention, the reductive disulfide bond conjugation between the light and heavy chains is preferred, which involves the attachment through the reaction of thiol groups (sulfur atoms of cysteine residues) formed after reducing one or more of disulfide bond sites between the light and heavy chains (two sites between the heavy chains, and two sites between the heavy and light chain).

Unless otherwise specified, the term “alkyl” refers to a monovalent saturated aliphatic hydrocarbon group, including a linear or branched group containing 1-20 carbon atoms, preferably containing 1-10 carbon atoms (namely C₁₋₁₀alkyl), further preferably containing 1-8 carbon atoms (C₁₋₈alkyl), more preferably containing 1-6 carbon atoms (namely C₁₋₆alkyl). For example “C₁₋₆alkyl” means that the group is an alkyl group, and the number of carbon atoms in the carbon chain is between 1 and 6 (specifically 1, 2, 3, 4, 5 or 6). Examples include, but are not limited to methyl, ethyl, n-propyl, isopropyl, n-butyl, isobutyl, tert-butyl, sec-butyl, n-pentyl, neopentyl, 1,1-dimethylpropyl, 1,2-dimethylpropyl, 2,2-dimethylpropyl, 1-ethylpropyl, 2-methylbutyl, 3-methylbutyl, n-hexyl and the like.

Unless otherwise specified, the term “about” means that the value falls within an acceptable error range of the specific value as determined by a person of ordinary skill in the art, which depends in part on how the value is measured or determined (i.e., the limitations of the measurement system). For example, each occurrence of “about” in the field to which the present invention belongs may imply a range of ±20%, ±15%, ±10%, +5%, or +1% of the specific value indicated thereafter.

### DESCRIPTION OF THE DRAWINGS

FIG. 1 shows the decection result for the DAR value of DP001-JSSW-001.

FIG. 2 shows the decection result for the DAR value of DP001-JSSW-001.

FIG. 3 shows the decection result for the DAR value of partitumab-JSSW-001.

FIG. 4 shows the decection result for the DAR value of SWY2110-JSSW-001.

FIG. 5 shows the decection result for the DAR value of SWY2111-JSSW-001.

FIG. 6 shows the decection result for the DAR value of SWY2112-JSSW-001.

FIG. 7 shows the decection result for the DAR value of SWY2113-JSSW-001.

FIG. 8 shows the decection result for the DAR value of partitumab-Dxd.

FIG. 9 shows the decection result for the DAR value of SWY2110-Dxd.

FIG. 10 shows the decection result for the DAR value of SWY2110-Vc MMAE.

FIG. 11 shows the effect of DP001-ADC on the tumor volume of HER2-expressing human breast cancer JIMT-1 xenograft tumor in NU/NU mice.

FIG. 12 shows the effect of patritumab-ADC on the tumor volume of human lung cancer PC9-GR (gefitinib-resistant cell) xenograft tumor in NU/NU mice.

FIG. 13 shows the effect of SWY2110-ADC on the tumor volume of human colorectal cancer DiFi xenograft tumor in NU/NU mice.

FIG. 14 shows the effect of SWY2110-ADC on the tumor volume of human lung cancer PC9-GR (gefitinib-resistant cell) xenograft tumor in NU/NU mice.

FIG. 15 shows the detection result of the inhibitory activity of SWY2110-ADC on DiFi cells.

FIG. 16 shows the detection result of the inhibitory activity of SWY2110-ADC on PC9-GR cells FIG. 17 shows the detection result of the bystander effect of DP001-ADC.

FIG. 18 shows the effect of SWY2110-ADC on the tumor volume of human lung cancer PC9-AR (PC9-Del19/T790M/C797S, osimertinib-resistant cell) xenograft tumor in NU/NU mice.

FIG. 19 shows the effect of SWY2110-ADC, SWY2111-ADC, SWY2112-ADC, SWY2113-ADC on the tumor volume of human lung cancer NCI-H1975 xenograft tumor in NU/NU mice.

FIG. 20 shows the effect of SWY2110-ADC, SWY2111-ADC, SWY2112-ADC, SWY2113-ADC on the tumor volume of human breast cancer MDA-MB-468 xenograft tumor in NOD-SCID mice.

FIG. 21 shows the effect of SWY2110-ADC, SWY2111-ADC, SWY2112-ADC, SWY2113-ADC on the tumor volume of human colorectal cancer DiFi xenograft tumor in NU/NU mice.

FIG. 22 shows the effect of SWY2110-ADC, SWY2111-ADC, SWY2112-ADC, SWY2113-ADC on the tumor volume of human lung cancer PC9-GR (gefitinib-resistant cell) xenograft tumor in NU/NU mice.

FIG. 23 shows the effect of patritumab-ADC on the tumor volume of human lung cancer PC9-DTC (Dell9/T790M/C797S, osimertinib-resistant cell) xenograft tumor in NOD-SCID mice.

FIG. 24 shows the effect of patritumab-ADC on the tumor volume of human colon cancer SW620 xenograft tumor in NU/NU mice.

FIG. 25 shows the detection result of the inhibitory activity of patritumab-ADC on DiFi cells.

### DETAILED DESCRIPTION

The present invention is further described below in connection with specific examples. It should be understood that these examples are intended to illustrate the present invention only and are not intended to limit the scope of the present invention. Experimental methods for which no specific conditions are indicated in the following examples are generally in accordance with conventional conditions or in accordance with the conditions recommended by the manufacturers. Unless otherwise defined, all professional and scientific terms used herein have the same meaning as those familiar to those skilled in the art. In addition, any methods and materials similar or equivalent to those described herein may be used in the methods of the present invention. The preferred embodiments and materials shown in this disclosure are illustrative only.

The structures of the compounds according to the present invention are determined by nuclear magnetic resonance (NMR) or/and liquid chromatograph mass spectrometry (LC-MS) or/and liquid chromatography (HPLC). The instrument used for NMR is Bruker Avance III 400 MHz nuclear magnetic resonance apparatus; the instrument used for LC-MS is SHIMADZU LC-20AD-PDA-LCMS-2020; and the instrument used for HPLC is SHIMADZU LC-20AD-PDA high performance liquid chromatograph.

The starting materials in the examples of the present invention are known and commercially available, or can be synthesised using those methods known in the art or according to those methods known in the art.

The antibodies of the present invention can be prepared by hybridoma technology described by Kohler et al., Nature (1975) for the first time, or by recombinant DNA method (U.S. Pat. No. 4,816,567, etc.).

### Preparation Example

An antibody having two heavy chains with the amino acid sequence represented by SEQ ID NO:9 and two light chains with the amino acid sequence represented by SEQ ID NO:10 was defined as DP001;

An antibody having two heavy chains with the amino acid sequence represented by SEQ ID NO: 19 and two light chains with the amino acid sequence represented by SEQ ID NO:20 was defined as patritumab;

An antibody having two heavy chains with the amino acid sequence represented by SEQ ID NO:29 and two light chains with the amino acid sequence represented by SEQ ID NO:30 was defined as SWY2110;

An antibody having two heavy chains with the amino acid sequence represented by SEQ ID NO:34 and two light chains with the amino acid sequence represented by SEQ ID NO:30 was defined as SWY2111;

An antibody having two heavy chains with the amino acid sequence represented by SEQ ID NO:37 and two light chains with the amino acid sequence represented by SEQ ID NO:30 was defined as SWY2112;

An antibody having two heavy chains with the amino acid sequence represented by SEQ ID NO:40 and two light chains with the amino acid sequence represented by SEQ ID NO:30 was defined as SWY2113.

### Antibody Sequence Description

1. HER2 antibody DP001 (trastuzumab)
Light chain (SEQ ID NO: 10):
DIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Heavy chain (SEQ ID NO: 9):
EVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYA
MDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHY
TQKSLSLSPGK
DP001 HCDR1: GFNIKDTYIH (SEQ ID NO: 1)
DP001 HCDR2: RIYPTNGYTRYAD (SEQ ID NO: 2)
DP001 HCDR3: WGGDGFYAMDY (SEQ ID NO: 3)
DP001 LCDR1: RASQDVNTAVA (SEQ ID NO: 4)
DP001 LCDR2: SASFLYS (SEQ ID NO: 5)
DP001 LCDR3: QQHYTTPPT (SEQ ID NO: 6)
DP001 HV: SEQ ID NO: 7
EVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYA
MDYWGQGTLVTVSS
DP001 LV: SEQ ID NO: 8
DIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIK
2. HER3 antibody patritumab
Light chain (SEQ ID NO: 20):
DIEMTQSPDSLAVSLGERATINCRSSQSVLYSSSNRNYLAWYQQNPGQPPKL
LIYWASTRESGVPDRFSGSGSGTDFTLTISSLQAEDVAVYYCQQYYSTPRTFGQGTK
VEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Heavy chain (SEQ ID NO: 19):
QVQLQQWGAGLLKPSETLSLTCAVYGGSFSGYYWSWIRQPPGKGLEWIGEI
NHSGSTNYNPSLKSRVTISVETSKNQFSLKLSSVTAADTAVYYCARDKWTWYFDL
WGRGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKS
CDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSL
SLSPGK
Patritumab HCDR1: GGSFSGYYWS SEQ ID NO: 11
Patritumab HCDR2: EINHSGSTNYNPSLKS SEQ ID NO: 12
Patritumab HCDR3: DKWTWYFDL SEQ ID NO: 13
Patritumab LCDRI: RSSQSVLYSSSNRNYLA SEQ ID NO: 14
Patritumab LCDR2: WASTRES SEQ ID NO: 15
Patritumab LCDR3: QQYYSTPRT SEQ ID NO: 16
Patritumab HV: SEQ ID NO: 17
QVQLQQWGAGLLKPSETLSLTCAVYGGSFSGYYWSWIRQPPGKGLEWIGEI
NHSGSTNYNPSLKSRVTISVETSKNQFSLKLSSVTAADTAVYYCARDKWTWYFDL
WGRGTLVTVSS
Patritumab LV: SEQ ID NO: 18
DIEMTQSPDSLAVSLGERATINCRSSQSVLYSSSNRNYLAWYQQNPGQPPKL
LIYWASTRESGVPDRFSGSGSGTDFTLTISSLQAEDVAVYYCQQYYSTPRTFGQGTK
VEIK
3. EGFR antibody SWY2110
Light chain (SEQ ID NO: 30):
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Heavy chain (SEQ ID NO: 29):
QVQLQESGPGLVKPSETLSLTCTVSGFSLSNYDVHWVRQAPGKGLEWLGVI
WSGGNTDYNTPFTSRLTISVDTSKNQFSLKLSSVTAADTAVYYCARALDYYDYEFA
YWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSPGK
SWY2110 HCDR1: NYDVH SEQ ID NO: 21
SWY2110 HCDR2: VIWSGGNTDYNTPFTS SEQ ID NO: 22
SWY2110 HCDR3: ALDYYDYEFAY SEQ ID NO: 23
SWY2110 LCDR1: RASQSIGTNIH SEQ ID NO: 24
SWY2110 LCDR2: YASESIS SEQ ID NO: 25
SWY2110 LCDR3: QQNNEWPTS SEQ ID NO: 26
SWY2110 HV: SEQ ID NO: 27
QVQLQESGPGLVKPSETLSLTCTVSGFSLSNYDVHWVRQAPGKGLEWLGVI
WSGGNTDYNTPFTSRLTISVDTSKNQFSLKLSSVTAADTAVYYCARALDYYDYEFA
YWGQGTLVTVSS
SWY2110 LV: SEQ ID NO: 28
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
4. EGFR antibody SWY2111
Light chain (SEQ ID NO: 30):
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Heavy chain (SEQ ID NO: 34):
QVQLQESGPGLVKPSETLSLTCTVSGFSLSDYDVHWVRQAPGKGLEWLGVI
WSGGNTDYNTPFTSRLTISVDTSKNQFSLKLSSVTAADTAVYYCARALDDYDYEFA
YWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSPGK
SWY2111 HCDR1: DYDVH SEQ ID NO: 31
SWY2111 HCDR2: VIWSGGNTDYNTPFTS SEQ ID NO: 22
SWY2111 HCDR3: ALDDYDYEFAY SEQ ID NO: 32
SWY2111 LCDR1: RASQSIGTNIH SEQ ID NO: 24
SWY2111 LCDR2: YASESIS SEQ ID NO: 25
SWY2111 LCDR3: QQNNEWPTS SEQ ID NO: 26
SWY2111 HV: SEQ ID NO: 33
QVQLQESGPGLVKPSETLSLTCTVSGFSLSDYDVHWVRQAPGKGLEWLGVI
WSGGNTDYNTPFTSRLTISVDTSKNQFSLKLSSVTAADTAVYYCARALDDYDYEFA
YWGQGTLVTVSS
SWY2111 LV: SEQ ID NO: 28
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
5. EGFR antibody SWY2112
Light chain (SEQ ID NO: 30):
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Heavy chain (SEQ ID NO: 37):
QVQLQESGPGLVKPSETLSLTCTVSGFSLSEYDVHWVRQAPGKGLEWLGVI
WSGGNTDYNTPFTSRLTISVDTSKNQFSLKLSSVTAADTAVYYCARALDDYDYEFA
YWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSPGK
SWY2112 HCDR1: EYDVH SEQ ID NO: 35
SWY2112 HCDR2: VIWSGGNTDYNTPFTS SEQ ID NO: 22
SWY2112 HCDR3: ALDDYDYEFAY SEQ ID NO: 32
SWY2112 LCDR1: RASQSIGTNIH SEQ ID NO: 24
SWY2112 LCDR2: YASESIS SEQ ID NO: 25
SWY2112 LCDR3: QQNNEWPTS SEQ ID NO: 26
SWY2112 HV: SEQ ID NO: 36
QVQLQESGPGLVKPSETLSLTCTVSGFSLSEYDVHWVRQAPGKGLEWLGVI
WSGGNTDYNTPFTSRLTISVDTSKNQFSLKLSSVTAADTAVYYCARALDDYDYEFA
YWGQGTLVTVSS
SWY2112 LV: SEQ ID NO: 28
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
6. EGFR antibody SWY2113
Light chain (SEQ ID NO: 30):
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Heavy chain (SEQ ID NO: 40):
QVQLQESGPGLVKPSETLSLTCTVSGFSLSHYDVHWVRQAPGKGLEWLGVI
WSGGNTDYNTPFTSRLTISVDTSKNQFSLKLSSVTAADTAVYYCARALDDYDYEFA
YWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSPGK
SWY2113 HCDR1: HYDVH SEQ ID NO: 38
SWY2113 HCDR2: VIWSGGNTDYNTPFTS SEQ ID NO: 22
SWY2113 HCDR3: ALDDYDYEFAY SEQ ID NO: 32
SWY2113 LCDR1: RASQSIGTNIH SEQ ID NO: 24
SWY2113 LCDR2: YASESIS SEQ ID NO: 25
SWY2113 LCDR3: QQNNEWPTS SEQ ID NO: 26
SWY2113 HV: SEQ ID NO: 39
QVQLQESGPGLVKPSETLSLTCTVSGFSLSHYDVHWVRQAPGKGLEWLGVI
WSGGNTDYNTPFTSRLTISVDTSKNQFSLKLSSVTAADTAVYYCARALDDYDYEFA
YWGQGTLVTVSS
SWY2113 LV: SEQ ID NO: 28
EIVLTQSPDFQSVTPKEKVTITCRASQSIGTNIHWYQQKPDQSPKLLIKYASE
SISGIPSRFSGSGSGTDFTLTINSLEAEDAATYYCQQNNEWPTSFGQGTKLEIK

### Example 1

### N-((2R,10S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-6-(2,5-dioxo-2,5-dihydro-1H-pyrrol-1-yl)hexanamide (JSSW-001)

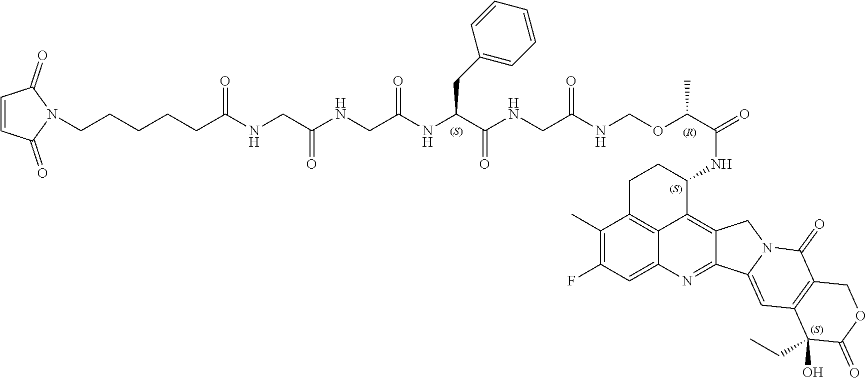

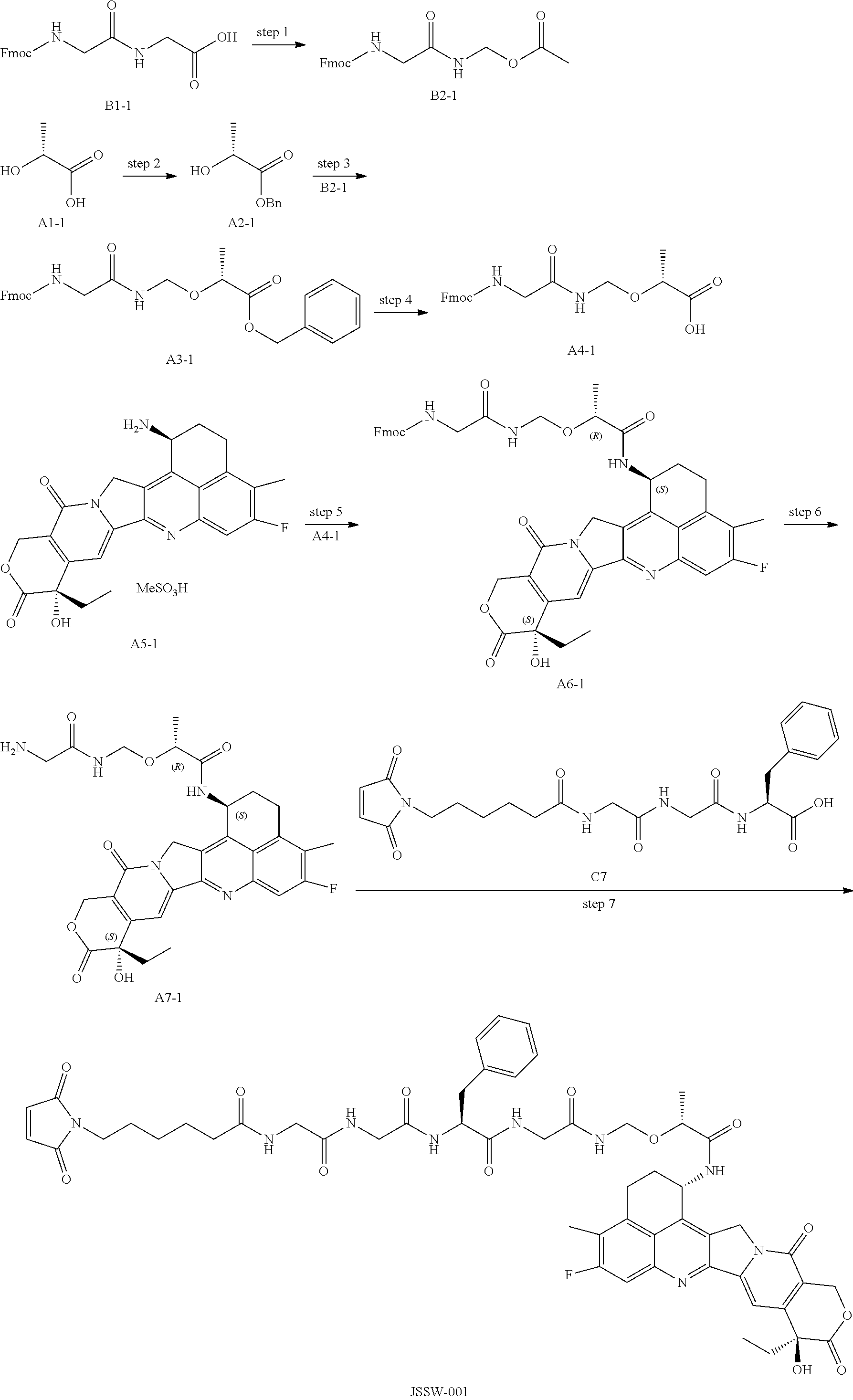

Step 1: Preparation of compound B2-1: B1-1 (1.0 g, 2.28 mmol), anhydrous tetrahydrofuran (30 mL), toluene (10 mL), pyridine (0.5 mL), and lead tetraacetate (1.83 g, 4.12 mmol) were added to a 100 mL three-neck flask. The solution turned orange and was heated under reflux. After one hour, the solution became colorless, and a white solid was formed. After a three-hour reaction, the solution was filtered using celite, the solid was rinsed with ethyl acetate, and the filtrate was dried using a rotary evaporator. The obtained crude product was purified by column chromatography to produce a solid (640 mg). LC-MS[M+Na]⁺: m/z 391.1.

Step 2: Preparation of compound A2-1: A1-1 (2.7 g, 30.0 mmol) and N,N-dimethylformamide (30 mL) were added to a 100 mL three-neck flask, followed by the addition of potassium carbonate (4.17 g, 30.0 mmol) and benzyl bromide (2.4 mL, 20.0 mmol). After reacting at room temperature for 14 hours, the reaction mixture was added dropwise into water. Extraction was carried out using ethyl acetate, and the organic layers were combined. The combined organic layers were washed with saturated brine, dried over anhydrous magnesium sulfate, filtered, and concentrated. The crude product was purified by column chromatography (PE/EA=3:1) to produce 2.61 g of the target compound as a colorless liquid.

¹H NMR (400 MHz, CDCl₃): δ 7.33-7.41 (m, 5H), 5.21 (s, 2H), 4.29-4.35 (m, 1H), 2.77 (d, J=5.6 Hz, 1H), 1.44 (d, J=6.8 Hz, 3H).

Step 3: Preparation of compound A3-1: A2-1 (720 mg, 4.0 mmol), B2-1 (368 mg, 1.0 mmol), and dichloromethane (5 mL) were added to a 100 mL three-neck flask. Pyridinium 4-methylbenzenesulfonate (75 mg, 0.30 mmol) was then added. The reaction mixture was heated to reflux overnight. After the reaction was complete, ethyl acetate (20 mL) was added to the reaction mixture, which was then washed with water three times and dried over anhydrous magnesium sulfate. The magnesium sulfate was removed by filtration, and the solution was concentrated. The obtained crude product was purified by column chromatography (PE/EA=2:1) to produce a white solid A3-1 (290 mg).

LC-MS[M+Na]⁺: m/z 511.1. ¹H NMR (400 MHz, CDCl₃): δ 7.77 (d, J=7.2 Hz, 2H), 7.58 (d, J=7.6 Hz, 2H), 7.40 (t, J=7.6 Hz, 2H), 7.26-7.37 (m, 6H), 6.70 (brs, 1H), 5.12-5.30 (m, 3H), 4.76-4.89 (m, 2H), 4.45 (d, J=6.8 Hz, 1H), 4.19-4.27 (m, 2H), 3.69-3.85 (m, 2H), 1.41 (d, J=6.8 Hz, 3H).

Step 4: Preparation of compound A4-1: A3-1 (291 mg, 0.60 mmol) dissolved in tetrahydrofuran:ethyl acetate (4 mL/2 mL) was added to a 100 mL three-neck flask, along with Pd/C (10% w/w, 60 mg). After purging with hydrogen gas three times, a hydrogen balloon was attached, and the mixture was stirred overnight. After the reaction was complete, the mixture was filtered through celite, rinsed with methanol, and the organic layer was concentrated to produce the product A4-1. LC-MS[M+Na]⁺: m/z 399.1.

Step 5: Preparation of compound A6-1: A4-1 (50 mg, 0.13 mmol, 1.3eq), exatecan mesylate (A5-1) (50 mg, 0.049 mmol, 1.0eq), 2-(7-azabenzotriazol-1-yl)-N,N,N′,N′-tetramethyluronium hexafluorophosphate (48 mg, 0.13 mmol, 1.3eq), and N,N-diisopropylethylamine (36 mg, 0.28 mmol, 3.0eq) were dissolved in N,N-dimethylformamide (2 mL) in a 25 mL single-neck flask. The mixture was stirred overnight at room temperature. It was then diluted with ethyl acetate and washed with semi-saturated citric acid, followed by washing with brine, sodium bicarbonate solution, and brine again. The organic layer was dried over anhydrous sodium sulfate, and filtered. The anhydrous sodium sulfate was removed and the filtrate was concentrated. Purification was carried out using prep-TLC with a developer system of (DCM/MeOH=15:1) to produce the product A6-1 (56 mg). LC-MS[M+H]⁺: m/z 816.3.

Step 6: Preparation of compound A7-1: A6-1 (56 mg, 0.069 mmol), N,N-dimethylformamide (2 mL), and piperidine (12 mg, 0.14 mmol) were added to a 25 mL single-neck flask and stirred for 2 hours. The solution was then concentrated to produce a white solid. LC-MS[M+H]⁺: m/z 594.3.

Step 7: Preparation of compound JSSW-001: A7-1 (0.069 mmol), C7 (66 mg, 0.14 mmol), 2-(7-azabenzotriazol-1-yl)-N,N,N′,N′-tetramethyluronium hexafluorophosphate (53 mg, 0.14 mmol), and N,N-diisopropylethylamine (36 mg, 0.28 mmol) were dissolved in N,N-dimethylformamide (2 mL) in a 25 mL single-neck flask. The mixture was stirred for 2 hours at room temperature. It was then diluted with ethyl acetate (30 mL) and washed with semi-saturated citric acid, followed by washing with brine, sodium bicarbonate solution, and brine again. The organic layer was dried over anhydrous sodium sulfate, and filtered. The anhydrous sodium sulfate was removed and the filtrate was concentrated. Purification was carried out using prep-TLC with a developer system of (DCM/MeOH=15:1) to produce the product JSSW-001 (21 mg).

LC-MS[M+H]⁺: m/z 1048. ¹H NMR (400 MHz, DMSO-d6): δ 8.63 (t, J=6.4 Hz, 1H), 8.53 (d, J=8.8 Hz, 1H), 8.28 (t, J=6.0 Hz, 1H), 8.03-8.10 (m, 2H), 7.98 (t, J=6.0 Hz, 1H), 7.77 (d, J=11.2 Hz, 1H), 7.30 (s, 1H), 7.14-7.25 (m, 5H), 6.99 (s, 2H), 6.51 (s, 1H), 5.56-5.62 (m, 1H), 5.40 (s, 2H), 5.08-5.23 (m, 2H), 4.66-4.71 (m, 1H), 4.42-4.56 (m, 2H), 4.09-4.15 (m, 1H), 3.54-3.76 (m, 7H), 3.09-3.27 (m, 1H), 2.97-3.02 (m, 1H), 2.67-2.77 (m, 1H), 2.38 (s, 3H), 2.16-2.21 (m, 2H), 2.05-2.10 (m, 2H), 1.83-1.89 (m, 2H), 1.37-1.46 (m, 7H), 1.14-1.33 (m, 4H), 0.87 (t, J=7.2 Hz, 3H).

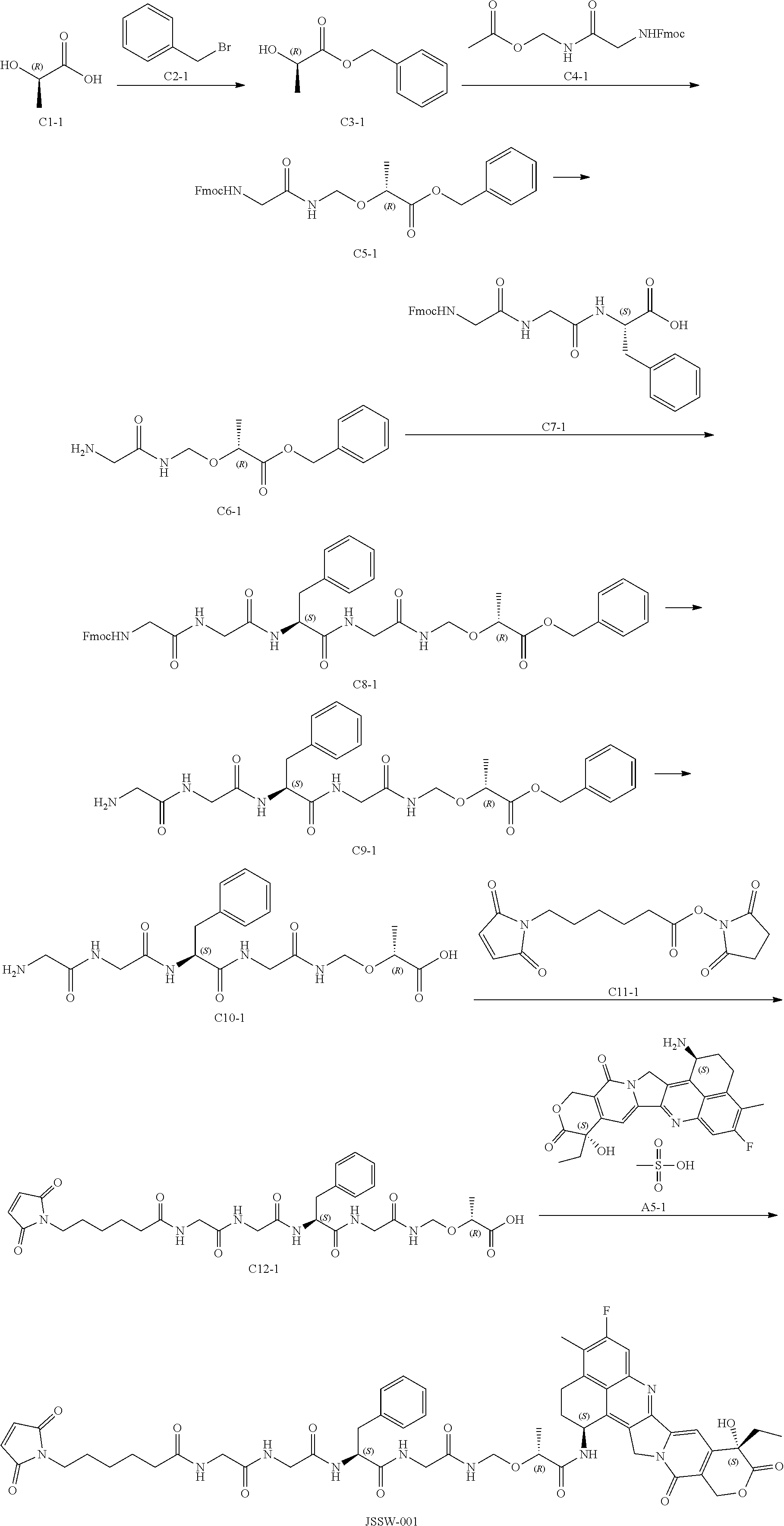

Step 1: Compound C1-1 (8 g, 88.86 mmol) and C2-1 (22.66 g, 133.33 mmol) were added to a 250 mL reaction flask, followed by the addition of dimethylacetamide (DMAc) (40 mL, 5 v/w). The mixture was stirred until dissolved completely, and the temperature was controlled to 0-10° C. Then, N,N-diisopropylethylamine (DIEA) (34.45 g, 266.6 mmol) was added dropwise. After the dropwise addition, the mixture was allowed to warm naturally to about 25° C. and stirred for 16 hours at this temperature. After TLC monitoring showed the complete reaction of the starting materials, the reaction mixture was poured into 120 mL of ice water. Methyl tert-butyl ether (MTBE) (40 mL) was added, and the mixture was stirred, allowed to stand for phase separation. The aqueous phase was extracted four times with MTBE (40 mL*4). The organic phases were combined and washed once with 0.5M hydrochloric acid solution (40 mL), once with water (40 mL), and twice with saturated brine (40 mL*2), dried over anhydrous sodium sulfate, filtered, concentrated, and purified by wet loading and column chromatography using a gradient system of petroleum ether:ethyl acetate=30:1→20:1→10:1 to produce a light yellow liquid C3-1. LC-MS[M+Na]⁺: m/z 203.

Step 2: Under nitrogen protection, C4-1 (6 g, 16.29 mmol, 1.0eq) and C3-1 (3.23 g, 17.92 mmol, 1.1 eq) were dissolved in tetrahydrofuran (THF) (60 mL, 10V/w). The temperature was lowered to 10-15° C., and p-toluenesulfonic acid (TsOH) (281 mg, 1.63 mmol, 0.1 eq) was added. After the completion of the addtion, the reaction was carried out at 14-18° C. for 4 hrs under TLC monitoring. After the reaction was complete, the reaction mixture was added to ice water (60 mL) and extracted with ethyl acetate (EA) (60 mL*3). The organic phases were combined and washed with saturated aqueous NaHCO₃ solution (60 mL), water (60 mL*2), and saturated brine (60 mL), dried over anhydrous sodium sulfate, filtered, concentrated by evaporating to dryness, and purified by column chromatography using a gradient system of n-hexane:ethyl acetate=10:1→5:1→3:1→2:1→1:1 to produce a colorless oily substance C5-1. LC-MS[M+Na]⁺: m/z 511.

Step 3: Under nitrogen protection, C5-1 (2.8 g, 5.74 mmol, 1.0eq) was dissolved in DMAc (28 mL, 10V/w). The temperature was lowered to 14-18° C., and 1,8-diazabicyclo[5.4.0]undec-7-ene (DBU) (436.6 mg, 2.87 mmol, 0.5eq) was added dropwise. The mixture was stirred at this temperature for 1.5 h. TLC monitoring showed the complete reaction of the starting materials. The temperature was then lowered to 0-10° C., and pyridinium p-toluenesulfonate (PPTS) (721.23 mg, 2.87 mmol, 0.5eq), 3-ethyl-1-(3-dimethylaminopropyl)carbodiimide hydrochloride (EDCI) (1.1 g, 5.74 mmol, 1.0eq), 1-hydroxybenzotriazole (HOBT) (775 mg, 5.74 mmol, 1.0eq), and C7-1 (2.45 g, 4.88 mmol, 0.85eq) were added. After the completion of the addition, the reaction was carried out at 0-10° C. for 3-4 hrs under TLC monitoring until C7-1 was completely reacted. The reaction mixture was added to ice water (100 mL) and extracted with 2-methyl tetrahydrofuran (2-Me THF) (100 mL*3). The organic phases were combined and washed with 0.5M hydrochloric acid (150 mL*2), saturated aqueous NaHCO₃ solution (100 mL*3), water (100 mL), and saturated brine (100 mL), dried over anhydrous sodium sulfate, filtered, concentrated by evaporating to dryness, and purified by column chromatography using a gradient system of DCM→DCM:MeOH=80:1→60:1→50:1→40:1→30:1→20:1 to produce a white foamy solid C8-1 (3.1 g, yield 72%). LC-MS[M+Na]⁺: m/z 772.

Step 4: Under nitrogen protection, C8-1 (3.1 g, 4.14 mmol, 1.0eq) was dissolved in dichloromethane (DCM) (46.5 mL, 15V/w) at a room temperature of 25-30° C. DBU (314.6 mg, 2.07 mmol, 0.5eq) was added dropwise, and the mixture was stirred at this temperature for 16 h. TLC monitoring showed the complete reaction of the starting materials. The reaction mixture was directly subjected to wet column chromatography using a gradient system of DCM→DCM:MeOH=50:1→30:1→20:1→10:1 to produce a white foamy solid C9-1. LC-MS[M+H]⁺: m/z 528.

Step 5: Under nitrogen protection, C9-1 (1.7 g, 3.2 mmol, 1.0eq) was dissolved in water (25.5 mL, 15V/w) and tert-butanol (8.5 mL, 5V/w). The mixture was stirred until completely dissolved. The atmosphere was replaced with nitrogen gas once, and Pd/C (0.34 g, 20% w/w) was added. The atmosphere was replaced with hydrogen gas three times. The hydrogenation reaction was carried out at a room temperature of 25-30° C. for 12 hrs. LCMS monitoring showed the complete reaction of the starting materials. The reaction mixture was filtered, and the filter cake was washed with water (25 mL*2). The filtrate was filtered again, and the tert-butanol was removed by concentration. The aqueous phase was directly lyophilized to produce a white powdery solid C10-1 (1.5 g in total, yield 100%). LC-MS[M+H]⁺: m/z 438.

Step 6: Under nitrogen protection, C10-1 (1.4 g, 3.2 mmol, 1.0eq) was dissolved in acetonitrile (14 mL, 10 V/w) and water (28 mL, 20 V/w). C11-1 (1.86 g, 6.097 mmol, 1.1 eq) was added, and the mixture was stirred until completely dissolved. The temperature was lowered to 0-10° C., and DIEA (330.24 mg, 2.56 mmol) was added dropwise. After the dropwise addition, the mixture was stirred to react at this temperature for 16 h. LC-MS monitoring showed the complete reaction of the starting materials. A buffer solution was prepared by dissolving Na₂HPO₄ (260 mg) and NaH₂PO₄ (5.6 g) in 20 mL of water under stirring, and the prepared buffer solution was cooled to 0-5° C. The reaction mixture was poured into the cooled buffer solution and extracted with DCM/isopropanol=4/1 (50 mL*4). The organic phases were combined, dried, filtered, concentrated by evaporating to dryness, and purified by column chromatography using a gradient system of DCM→DCM:MeOH=50:1→30:1→20:1→15:1 to produce a yellow solid C12-1.

Step 7: Under nitrogen protection, A5-1 (547.4 mg, 1.03 mmol, 1.0eq) was dissolved in 5% anhydrous sodium sulfate in water (6.5 mL) and THF (7.8 mL). The mixture was stirred but did not dissolve completely. The temperature was lowered to 0-10° C. Then, N-methylmorpholine (NMM) (104 mg, 1.03 mmol, 1.0eq) was added. After the completion of the addtion, the reaction was allowed to proceed at this temperature for 1 hour. C12-1 (650 mg, 1.03 mmol, 1.1 eq), EDCI (296.2 mg, 1.545 mmol, 1.5eq), and ethyl cyanoglyoxylate-2-oxime (73.9 mg, 0.52 mmol, 0.5eq) were added sequentially. After the completion of the addition, the reaction was allowed to proceed at 0-10° C. for 4-5 hours. TLC monitoring showed the complete reaction of the starting materials. The reaction mixture was then added to 0.1M hydrochloric acid (13 mL, 20V/w) in an ice bath, extracted with 2-methyl tetrahydrofuran (2-Me THF) (13 mL*3), and the organic phases were combined, washed once with 0.05M hydrochloric acid (13 mL, 20V/w) and twice with water (13 mL*2), then dried, filtered, concentrated by evaporating to dryness, and purified by column chromatography using a gradient system of DCM→DCM:MeOH=50:1→30:1→20:1→15:1→12:1 to produce a yellow foamy solid (JSSW-001). LC-MS[M+H]⁺: m/z 1048.

### Example 2 Antibody-Drug Conjugate (DP001-JSSW-001 ADC, DAR=4.0)

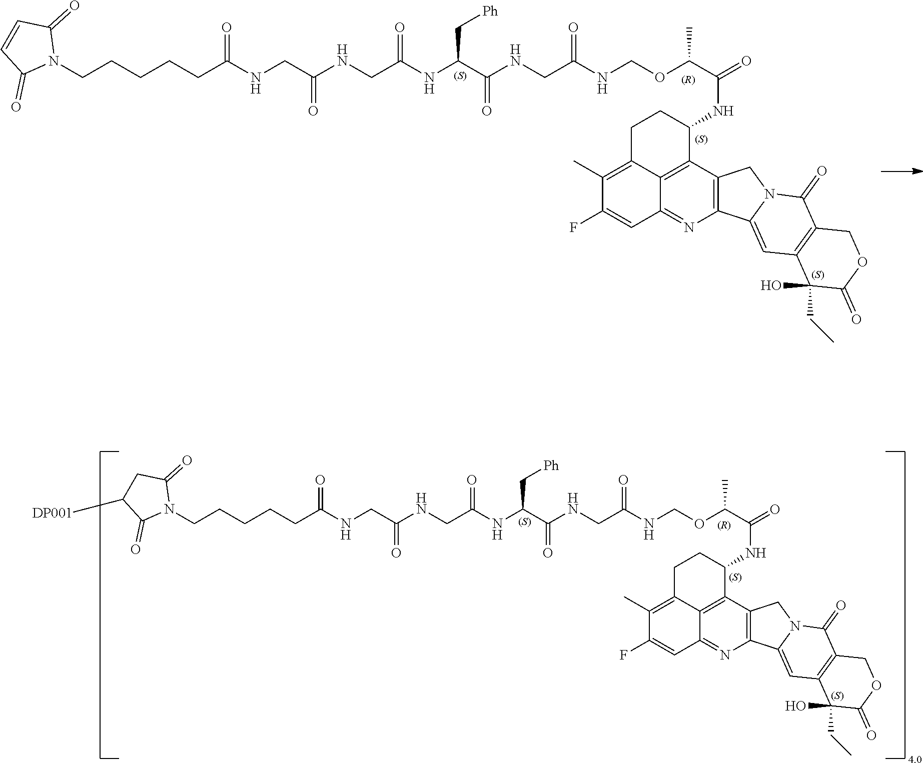

### Step 1. Reduction of Antibody:

The DP001 antibody medium, which was obtained by purifiction after the expression of plasmids encoding light and heavy chain genes that were transiently transfected into HEK293 cells using known technology, was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (17 μL; 2.5 equivalents per antibody molecule) and 1M aqueous dipotassium hydrogen phosphate solution (Tianjin Guangfu Science and Technology Development Co., Ltd., 50 μL). After confirming that the pH of the solution was within 7.4±0.1, the solution was incubated at 37° C. for 1 hour to reduce the disulfide bonds in the antibody.

### Step 2. Conjugation of Antibody and Linker-Drug Compound:

To the above solution was added a 10 mM solution of the compound JSSW-001 obtained in Example 1 in dimethylsulfoxide (31.28 μL; 4.6 equivalents per antibody molecule) at room temperature, and the mixture was mixed evenly and reacted at room temperature for 30 minutes to attach the linker-drug compound to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (DP001-JSSW-001 ADC, DAR=4.0).

### Step 4. Determination of Drug-Antibody Ratio (DAR) of Antibody-Drug Conjugate:

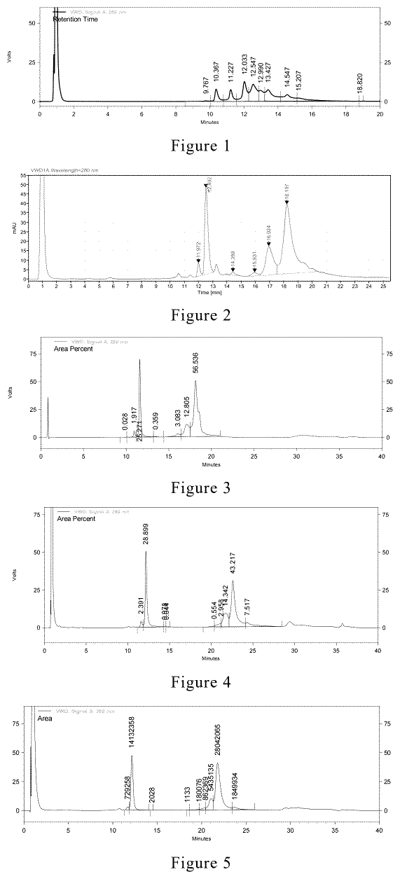

Dithiothreitol with a final concentration of 20 mM was added to 2 mg/kg antibody-drug-conjugate, and the mixture was incubated in a 37° C. water bath for 30 minutes. The sample, in which the disulfide bonds between antibody-drug conjugate chains were cleaved, was then used for HPLC analysis. The used HPLC system was the Agilent Technologies 1260 Infinity HPLC, and the used chromatograph column was the PLRP-S column (5 μm particle size; 2.1 mm×50 mm; Agilent Technologies). The column temperature was set at 80° C. The mobile phase A was 0.1% trifluoroacetic acid (TFA) in water, and the mobile phase B was 0.1% TFA in acetonitrile. The injection volume was 10 μL, and the gradient program was as follows: 0-3 minutes at 27%-27%, 3-8 minutes at 27%-35%, 8-25 minutes at 35%-43%, 25-26 minutes at 43%-95%, 26-31 minutes at 95%-95%, 31-32 minutes at 95%-27%, and 32-40 minutes at 27%-27%. Compared to the unconjugated light chain (L0) and heavy chain (H0), the hydrophobicity increased with the number of conjugated drugs for the drug-conjugated light chain (L1, conjugated with one drug) and heavy chains (H1, conjugated with one drug; H2, conjugated with two drugs; H3, conjugated with three drugs). Therefore, the elution followed the order of L0, L1, H0, H1, H2, and H3. The DAR value was calculated based on the peak area at 280 nm. A result of the DAR value is shown in FIG. 1.

$$DAR = ( \frac{L 1}{L 0 + L 1} \times 1 + \frac{H 1}{H 0 + H 1 + H 2 + H 3} \times 1 + \frac{H 2}{H 0 + H 1 + H 2 + H 3} \times 2 + \frac{H 3}{H 0 + H 1 + H 2 + H 3} \times 3 ) \times 2$$

### Example 3: Antibody-Drug Conjugate (DP001-JSSW-001 ADC, DAR=7.32)

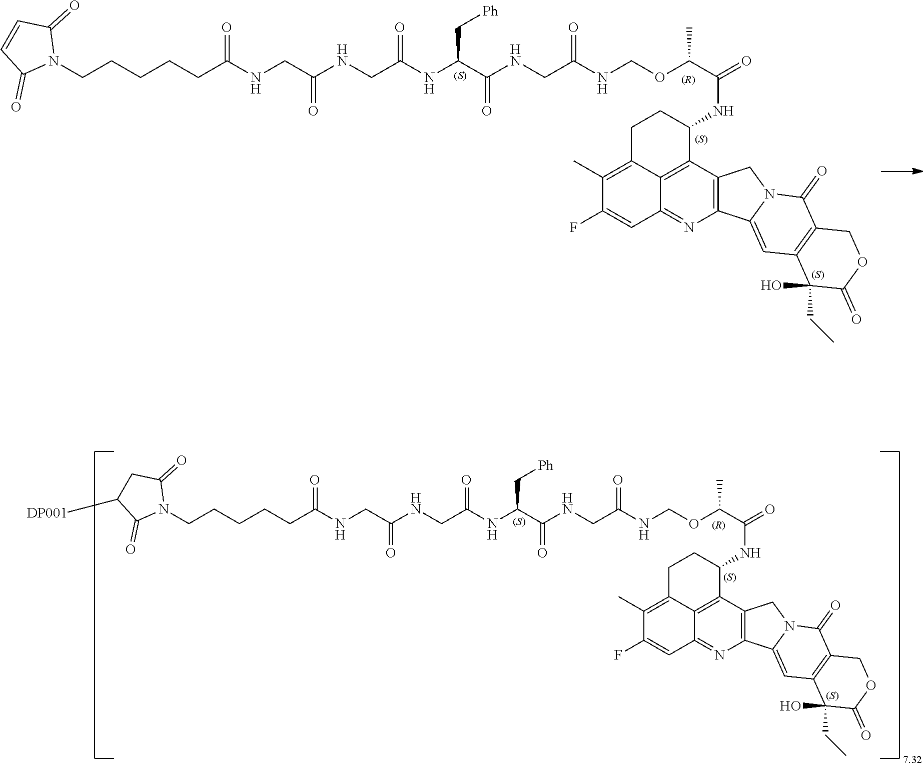

### Step 1. Reduction of Antibody:

The DP001 antibody medium, which was obtained by purification after the expression of plasmids encoding light and heavy chain genes that were transiently transfected into HEK293 cells using known technology, was exchanged with PBS6.0/EDTA to prepare an antibody solution with a concentration of 10 mg/mL. This solution (1.0 mL) was put into a 1.5 mL polypropylene tube, to which were added 100 mM aqueous TCEP (Bailingwei Science and Technology Co., Ltd.) solution (6.66 μL; 10 equivalents per antibody molecule) and 1M dipotassium hydrogen phosphate aqueous solution (Tianjin Guangfu Science and Technology Development Co., Ltd., 18 μL). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of the compound JSSW-001 obtained in Example 1 in dimethylsulfoxide (87 μL; 13 equivalents per antibody molecule) at room temperature, and the mixture was mixed evenly and reacted at room temperature for 30 minutes to attach the linker-drug to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (DP001-JSSW-001 ADC, DAR=7.32). The DAR value determination method was identical to that in step 4 of Example 2. The DAR value result is shown in FIG. 2.

### Example 4 Antibody-Drug Conjugate (Patritumab-JSSW-001 ADC, DAR=7.36)

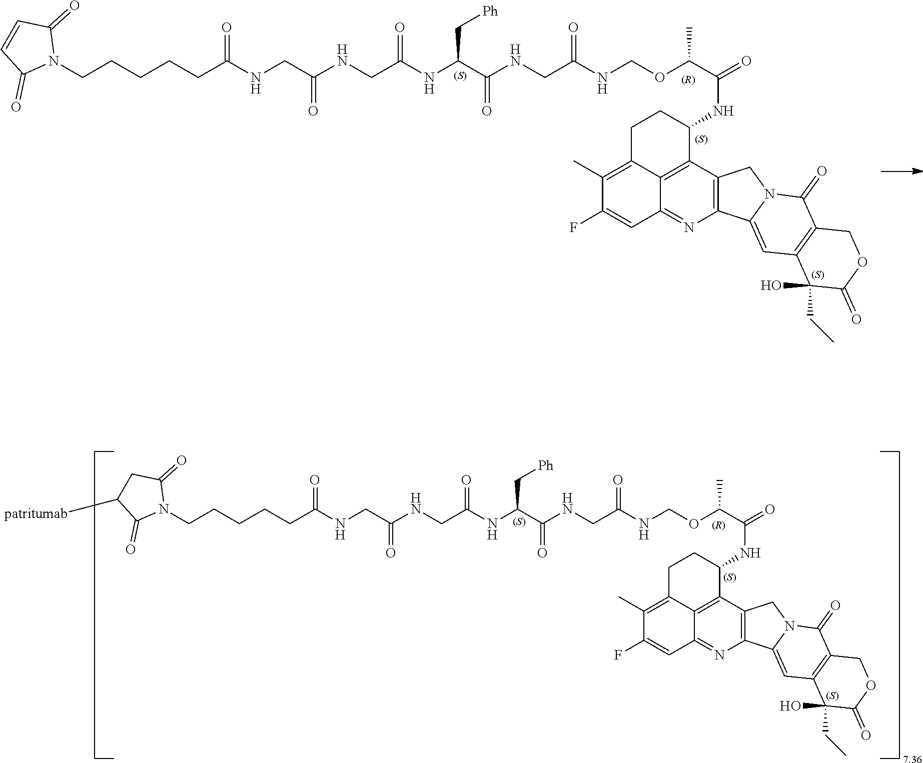

### Step 1. Reduction of Antibody:

The patritumab antibody medium, which was obtained by purification after the expression of plasmids encoding light and heavy chain genes that were transiently transfected into HEK293 cells using known technology, was exchanged with PBS6.0/EDTA to prepare a solution with a concentration of 10 mg/mL. This solution (1.0 mL) was put into a 1.5 mL polypropylene tube, to which were added 100 mM aqueous TCEP (Bailingwei Science and Technology Co., Ltd.) solution (6.66 μL; 10 equivalents per antibody molecule) and 1M dipotassium hydrogen phosphate aqueous solution (Tianjin Guangfu Science and Technology Development Co., Ltd., 18 μL). After confirming that the pH of the solution was within 7.0±0.1, the solution was incubated at 37° C. for 3 hours to reduce the disulfide bonds in the antibody.

### Step 2: Conjugation of Antibody and Linker-Drug Compound:

To the above solution was added a 10 mM solution of the compound JSSW-001 obtained in Example 1 in dimethylsulfoxide (87 μL; 13 equivalents per antibody molecule) at room temperature, and the mixture was mixed evenly and reacted at room temperature for 30 minutes to attach the linker-drug to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging factor greater than 1000 times. Thus, the non-conjugated linker-drug compound and other low molecular weight reagents were removed to obtain a purified antibody-drug conjugate (patritumab-JSSW-001 ADC, DAR=7.36). The DAR value determination method was identical to that in step 4 of Example 2. The DAR value result is shown in FIG. 3.

### Example 5 Antibody-Drug Conjugate (SWY2110-JSSW-001 ADC, DAR=7.12)

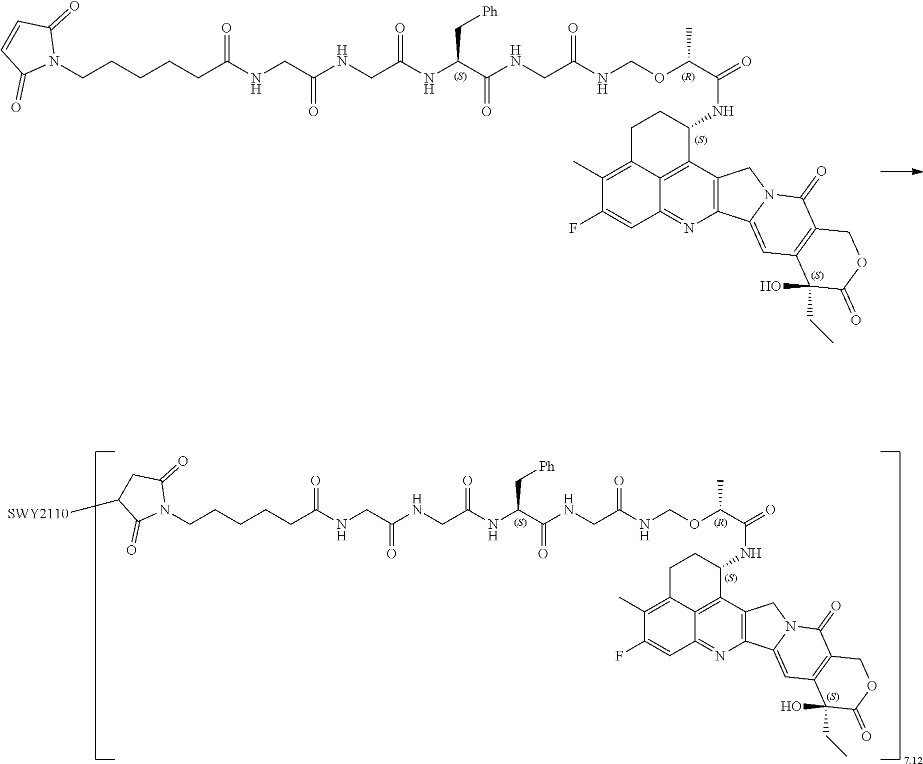

### Step 1. Reduction of Antibody:

The SWY2110 antibody medium, which was obtained by purification after the expression of plasmids encoding light and heavy chain genes that were transiently transfected into HEK293 cells using known technology, was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (66.6 μL; 10 equivalents per antibody molecule) and 1M aqueous dipotassium hydrogen phosphate solution (Tianjin Guangfu Science and Technology Development Co., Ltd.). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of the compound JSSW-001 obtained in Example 1 in dimethylsulfoxide (87 μL; 13 equivalents per antibody molecule) at room temperature, and the mixture was mixed evenly and reacted at room temperature for 30 minutes to attach the linker-drug to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution were added L-histidine 0.89 mg/ml and L-histidine hydrochloride 4.04 mg/ml as buffer, Tween80 0.03%, and sucrose 90 mg/ml, at pH=5.5. The non-conjugated linker-drug and other low molecular weight reagents were removed to produce a purified antibody-drug conjugate (SWY2110-JSSW-001 ADC, DAR=7.12). The DAR value determination method was identical to that in step 4 of Example 2. The DAR value result is shown in FIG. 4.

### Example 6 Preparation of pH-Dependent Antibodies

SWY2110 and EGFR protein were modelled and docked using computer simulation-assisted technology to obtain an antigen-antibody complex. Amino acids within 3A of the complex underwent D, E, and H scanning, which produced several pH-dependent antibodies that had a reduced affinity at pH=7.4 but basically had an unchanged affinity or had a lower decrease in affinity at pH=6.0. Several obtained antibody sequence genes were cloned into mammalian expression vectors and expressed through transfection of HEK-293 cells. After the completion of the expression, the expression supernatants were collected, and the antibodies were purified using an AKTA system equipped with Protein A pre-packed column to produce the desired antibodies.

### Example 7: Antibody Affinity Determination

MDA-MB-468 cells were digested into single cells using 0.25% trypsin-EDTA and divided equally into multiple aliquots, with each aliquot containing more than 10⁶ cells. SWY2110 (control) and antibodies obtained from Example 9 (diluted with PBS buffer solutions at pH=7.4 and pH 6.0, respectively) were added to the cells at 300 μL with serial 5-fold gradient dilutions. The mixtures were incubated at 4° C. for 1 hour and then centrifuged at 1000 rpm for 4 minutes to remove the supernatant. The cells were washed twice with PBS solution, followed by the addition of 200 μL of FITC-labelled mouse anti-human Fc secondary antibody with a dilution factor of 1:200. The mixtures were incubated at 4° C. for another hour, and then centrifuged at 1000 rpm for 4 minutes to remove the supernatant. Fluorescence intensity was detected using flow cytometry, and data were saved and analyzed using relevant software. The results of the antibody affinity determination after screening are shown in Table 1. The affinity determination results of the optimized antibodies with target antigens after the pH-dependent optimization showed that at pH=7.4, the affinity of the optimized antibodies SWY2111, SWY2112, and SWY2113 decreased significantly (about 3.0 to 9.7 times) compared to SWY2110; and at pH=6.0, the affinity of SWY2111 and SWY2112 remained basically unchanged (1.2 to 1.3 times) compared to SWY2110, while the affinity of the antibody SWY2113 decreased by 6.6 times.

TABLE 1
Result of Antibody Affinity Determination

| FACS (EC₅₀) | SWY2110 | SWY2111 | SWY2112 | SWY2113 |
|---|---|---|---|---|
| pH 7.4(nM) | 0.49 | 1.45 | 2.35 | 4.76 |
| pH 6.0(nM) | 0.51 | 0.60 | 0.67 | 3.39 |

### Example 8 Antibody-Drug Conjugate (SWY2111-JSSW-001 ADC, DAR=7.48)

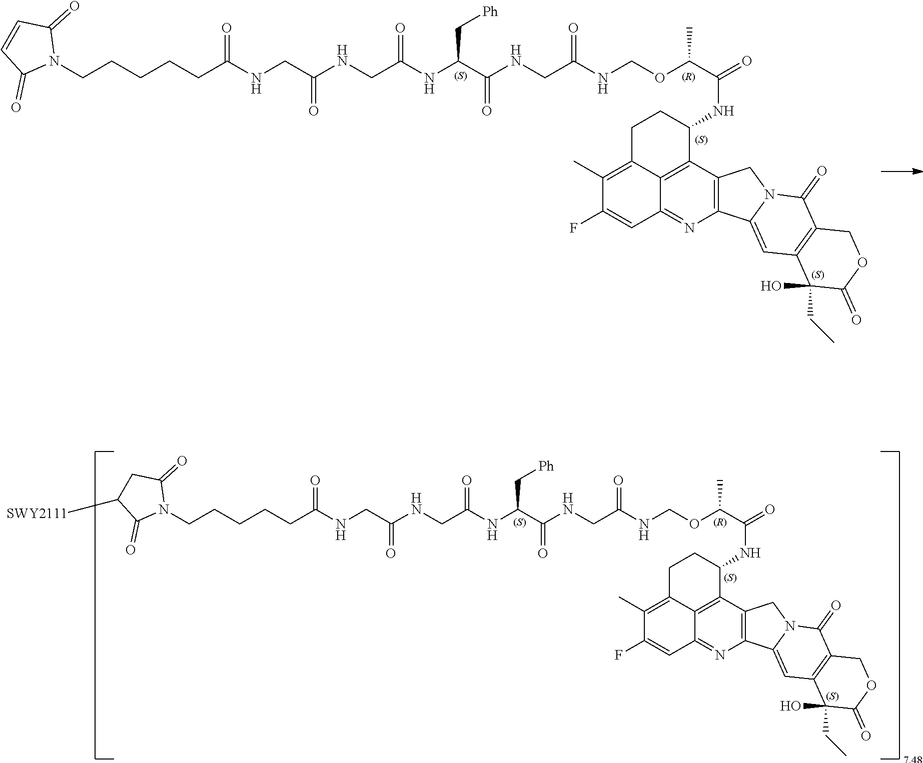

### Step 1. Reduction of Antibody:

The obtained SWY2111 antibody medium was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (66.6 μL; 10 equivalents per antibody molecule) and 1M aqueous dipotassium hydrogen phosphate solution (Tianjin Guangfu Science and Technology Development Co., Ltd.). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of the compound JSSW-001 obtained in Example 1 in dimethylsulfoxide (87 μL; 13 equivalents per antibody molecule) at room temperature, and the mixture was mixed evenly and reacted at room temperature for 30 minutes to attach the linker-drug to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution were added L-histidine 0.89 mg/ml and L-histidine hydrochloride 4.04 mg/ml as buffer, Tween80 0.03%, and sucrose 90 mg/ml, at pH=5.5. The non-conjugated linker-drug and other low molecular weight reagents were removed to produce a purified antibody-drug conjugate (SWY2111-JSSW-001 ADC, DAR=7.48). The DAR value determination method was identical to that in step 4 of Example 2. The DAR value result is shown in FIG. 5.

### Example 9 Antibody-Drug Conjugate (SWY2112-JSSW-001 ADC, DAR=7.51)

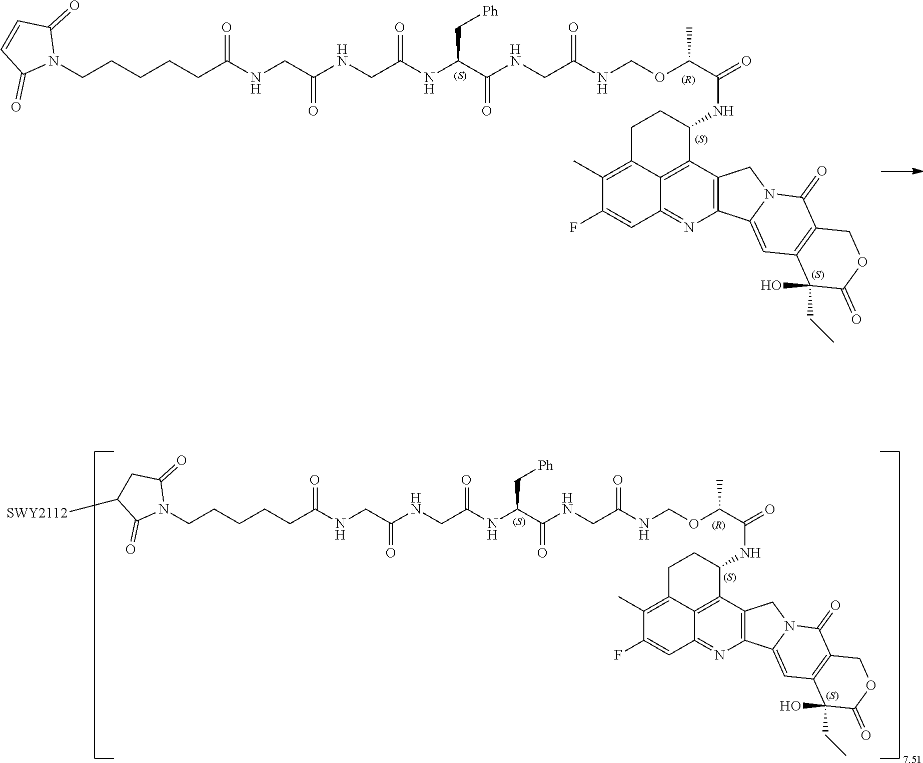

### Step 1. Reduction of Antibody:

The obtained SWY2112 antibody medium was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (66.6 μL; 10 equivalents per antibody molecule) and 1M aqueous dipotassium hydrogen phosphate solution (Tianjin Guangfu Science and Technology Development Co., Ltd.). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of the compound JSSW-001 obtained in Example 1 in dimethylsulfoxide (87 μL; 13 equivalents per antibody molecule) at room temperature, and the mixture was mixed evenly and reacted at room temperature for 30 minutes to attach the linker-drug to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

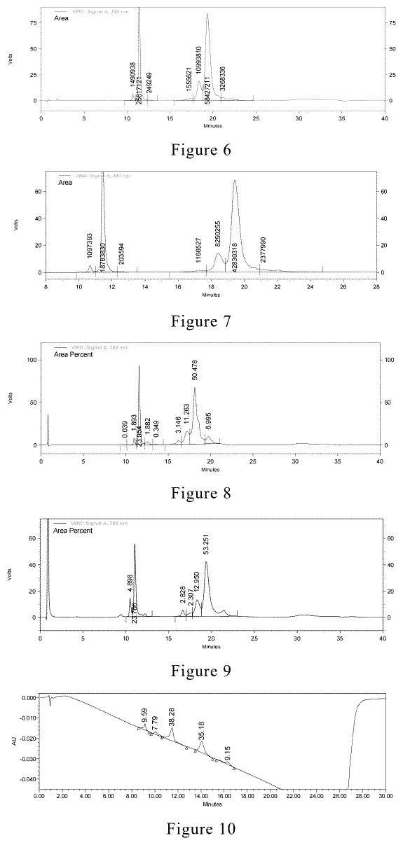

The reaction solution was purified by ultrafiltration using ultrafiltration centrifuge tubes (Merck, Ultracel® Regenerated Cellulose (30 kDa MWCO), 15 mL sample volume). To the reaction solution were added L-histidine 0.89 mg/ml and L-histidine hydrochloride 4.04 mg/ml as buffer, Tween80 0.03%, and sucrose 90 mg/ml, at pH=5.5. The non-conjugated linker-drug and other low molecular weight reagents were removed to produce a purified antibody-drug conjugate (SWY2112-JSSW-001 ADC, DAR=7.51). The DAR value determination method was identical to that in step 4 of Example 2. The DAR value result is shown in FIG. 6.

### Example 10 Antibody-Drug Conjugate (SWY2113-JSSW-001 ADC, DAR=7.50)

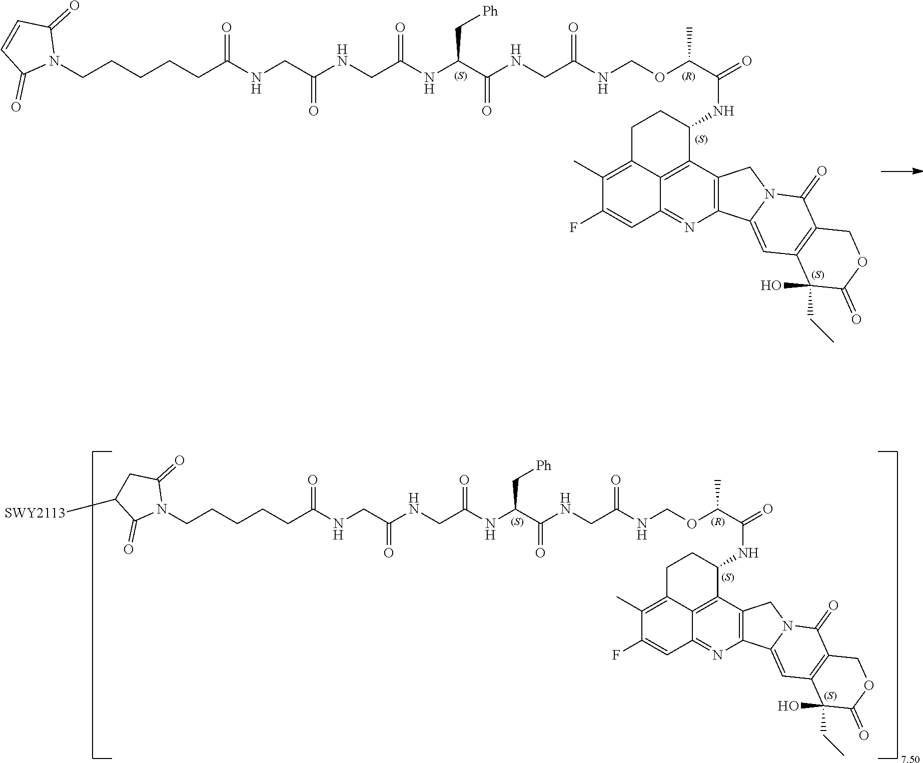

### Step 1. Reduction of Antibody:

The obtained SWY2113 antibody medium was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (66.6 μL; 10 equivalents per antibody molecule) and 1M aqueous dipotassium hydrogen phosphate solution (Tianjin Guangfu Science and Technology Development Co., Ltd.). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of the compound JSSW-001 obtained in Example 1 in dimethylsulfoxide (87 μL; 13 equivalents per antibody molecule) at room temperature, and the mixture was mixed evenly and reacted at room temperature for 30 minutes to attach the linker-drug to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution were added L-histidine 0.89 mg/ml and L-histidine hydrochloride 4.04 mg/ml as buffer, Tween80 0.03%, and sucrose 90 mg/ml, at pH=5.5. The non-conjugated linker-drug and other low molecular weight reagents were removed to produce a purified antibody-drug conjugate (SWY2113-JSSW-001 ADC, DAR=7.50). The DAR value determination method was identical to that in step 4 of Example 2. The DAR value result is shown in FIG. 7.

### Reference Example 1 Antibody-Drug Conjugate (DP001-DXD ADC, DAR=4.3)

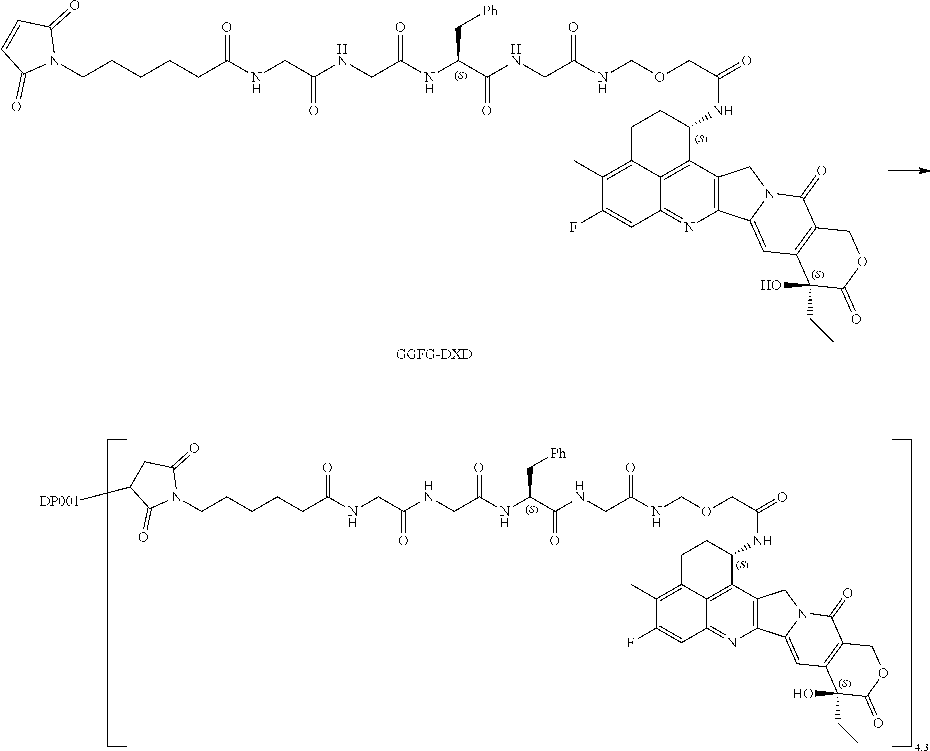

### Step 1. Reduction of Antibody:

The DP001 antibody medium, which was obtained by purification after the expression of plasmids encoding light and heavy chain genes that were transiently transfected into HEK293 cells using known technology, was exchanged with PBS6.0/EDTA to prepare a solution with a concentration of 10 mg/mL. This solution (1.0 mL) was put into a 1.5 mL polypropylene tube, to which were added 10 mM aqueous TCEP (Bailingwei Science and Technology Co., Ltd.) solution (17 μL; 2.5 equivalents per antibody molecule) and 1M aqueous dipotassium hydrogen phosphate solution (Tianjin Guangfu Science and Technology Development Co., Ltd., 50 μL). After confirming that the pH of the solution was within 7.4±0.1, the solution was incubated at 37° C. for 1 hour to reduce the disulfide bonds in the antibody.

### Step 2. Conjugation of Antibody and Linker-Drug Compound:

To the above solution was added a 10 mM solution of commercially available Deruxtecan (CAS No.: 1599440-13-7) in dimethylsulfoxide (31.28 μL; 4.6 equivalents per antibody molecule) at room temperature, and the mixture was mixed evenly and reacted at room temperature for 30 minutes to attach the linker-drug compound to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (DP001-DXD ADC, DAR=4.3). The method for measuring the drug-antibody ratio (DAR) of the antibody-drug conjugate was identical to that in step 4 of Example 2.

### Reference Example 2 Antibody-Drug Conjugate (DP001-DXD ADC, DAR=7.0)

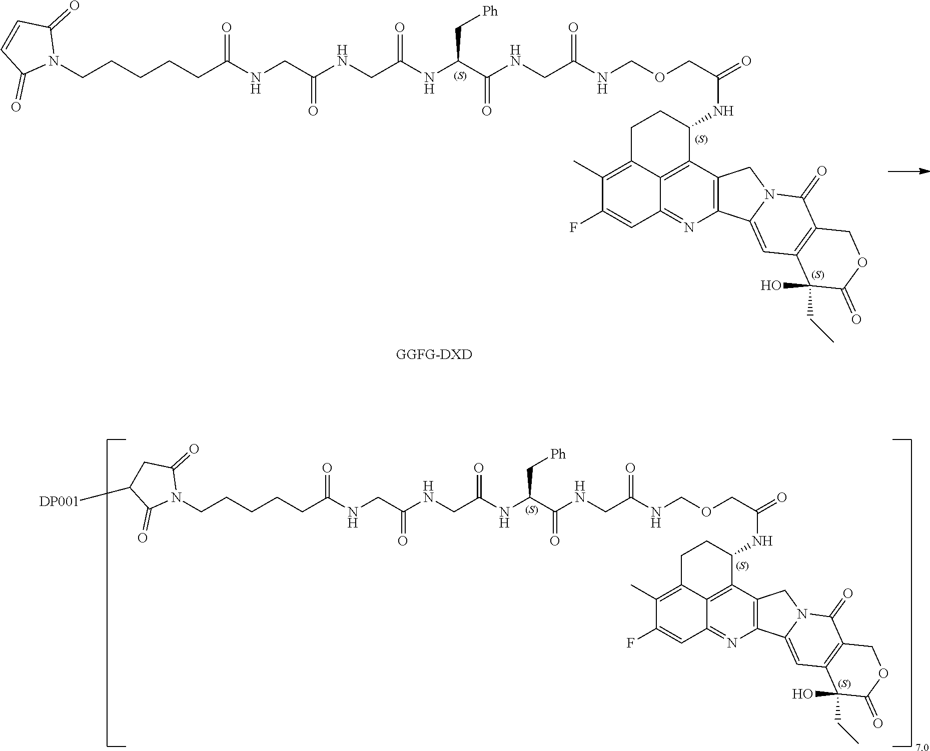

### Step 1. Reduction of Antibody:

The DP001 antibody medium was exchanged with PBS6.0/EDTA to prepare a solution having an antibody concentration of 10 mg/mL. This solution (1.0 mL) was put into a 1.5 mL polypropylene tube, to which were added 100 mM aqueous TCEP (Bailingwei Science and Technology Co., Ltd.) solution (6.66 μL; 10 equivalents per antibody molecule) and 1M dipotassium hydrogen phosphate aqueous solution (Tianjin Guangfu Science and Technology Development Co., Ltd., 18 μL). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of commercially available Deruxtecan (CAS No.:1599440-13-7) in dimethylsulfoxide (87 μL; 13 equivalents per antibody molecule) at room temperature, and the mixture was mixed evenly and reacted at room temperature for 30 minutes to attach the linker-drug compound to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel@Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (DP001-DXD ADC, DAR=7.0). The method for measuring the drug-antibody ratio (DAR) of the antibody-drug conjugate was identical to that in step 4 of Example 2.

### Reference Example 3 Antibody-Drug Conjugate (Patritumab-DXD ADC, DAR=7.3)

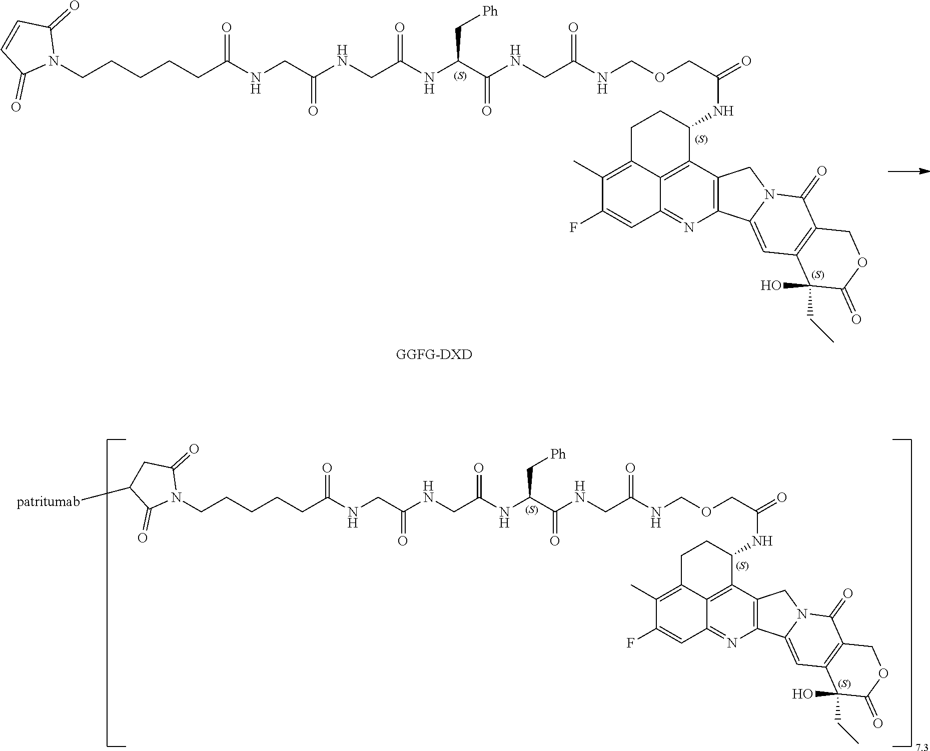

### Step 1. Reduction of Antibody:

The patritumab antibody medium, which was obtained by purification after the expression of plasmids encoding light and heavy chain genes that were transiently transfected into HEK293 cells using known technology, was exchanged with PBS6.0/EDTA to prepare an antibody solution with a concentration of 10 mg/mL. This solution (1.0 mL) was put into a 1.5 mL polypropylene tube, to which were added 100 mM aqueous TCEP (Bailingwei Science and Technology Co., Ltd.) solution (6.66 μL; 10 equivalents per antibody molecule) and 1M dipotassium hydrogen phosphate aqueous solution (Tianjin Guangfu Science and Technology Development Co., Ltd., 18 μL). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of commercially available Deruxtecan (CAS No.:1599440-13-7) in dimethylsulfoxide (87 μL; 13 equivalents per antibody molecule) at room temperature, and the mixture was mixed evenly and reacted at room temperature for 30 minutes to attach the linker-drug compound to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (patritumab-DXD ADC, DAR=7.3). The DAR value determination method was identical to that in step 4 of Example 2. The DAR value result is shown in FIG. 8.

### Reference Example 4 Antibody-Drug Conjugate (SWY2110-DXD ADC, DAR=6.93)

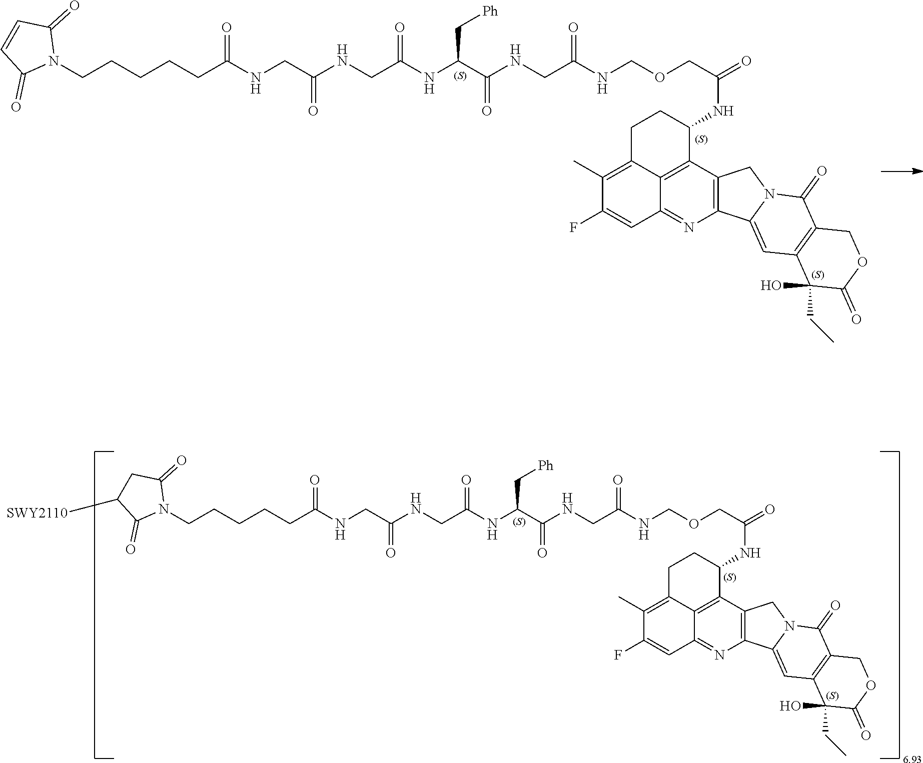

### Step 1. Reduction of Antibody:

The SWY2110 antibody medium, which was obtained by purification after the expression of plasmids encoding light and heavy chain genes that were transiently transfected into HEK293 cells using known technology, was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (66.6 μL; 10 equivalents per antibody molecule) and 1M aqueous dipotassium hydrogen phosphate solution (Tianjin Guangfu Science and Technology Development Co., Ltd.). After confirming that the pH of the solution was within 7.0±0.1, the solution was incubated at 37° C. for 3 hours to reduce the disulfide bonds in the antibody.

### Step 2. Conjugation of Antibody and Linker-Drug Compound:

To the above solution was added a 10 mM solution of commercially available Deruxtecan (CAS No.:1599440-13-7) in dimethylsulfoxide (87 μL; 13 equivalents per antibody molecule) at room temperature, and the mixture was mixed evenly and reacted at room temperature for 30 minutes to attach the linker-drug compound to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (SWY2110-DXD ADC, DAR=6.93). The DAR value determination method was identical to that in step 4 of Example 2. The DAR value result is shown in FIG. 9.

### Reference Example 5 Antibody-Drug Conjugate (SWY2110-VC MMAE ADC, DAR=4.53)

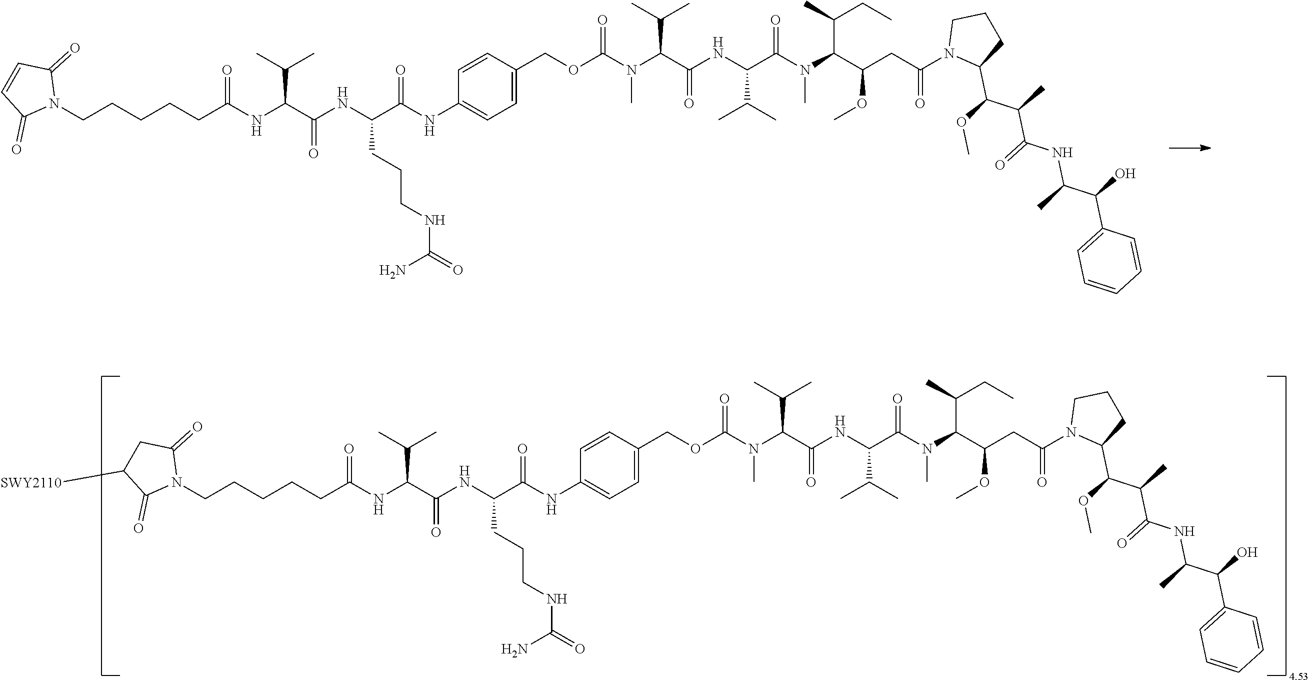

### Step 1. Reduction of Antibody:

The SWY2110 antibody medium, which was obtained by purification after the expression of plasmids encoding light and heavy chain genes that were transiently transfected into HEK293 cells using known technology, was exchanged with PBS6.0/EDTA to prepare an antibody solution with a concentration of 10 mg/mL. This solution (1.0 mL) was put into a 1.5 mL polypropylene tube, to which were added 10 mM aqueous TCEP (Bailingwei Science and Technology Co., Ltd.) solution (17 μL; 2.5 equivalents per antibody molecule) and 1M aqueous dipotassium hydrogen phosphate solution (Tianjin Guangfu Science and Technology Development Co., Ltd.). After confirming that the pH of the solution was within 7.4±0.1, the solution was incubated at 37° C. for 1 hour to reduce the disulfide bonds in the antibody.

### Step 2. Conjugation of Antibody and Linker-Drug Compound:

To the above solution was added a 10 mM solution of commercially available mc-vc-PAB-MMAE (CAS No.:646502-53-6, abbrev. Vc MMAE) in dimethylsulfoxide (31.28 μL; 4.6 equivalents per antibody molecule) at room temperature, and the mixture was mixed evenly and reacted at room temperature for 30 minutes to attach the linker-drug compound to the antibody. Next, 100 mM aqueous NAC (Bailingwei Science and Technology Co., Ltd.) solution (10.2 μL) was added, and the mixture was further stirred at room temperature for 20 minutes to terminate the reaction.

### Step 3. Purification of Antibody-Drug Conjugate:

The reaction solution was purified by ultrafiltration using ultrafiltration centrifuge tubes (Merck, Ultracel® Regenerated Cellulose (30 kDa MWCO), 15 mL sample volume). To the reaction solution was added 25 mM 2-(N-morpholine)ethanesulfonic acid (MES) as purification buffer, pH=6.5. The resulting solution was concentrated to 1-2 mL, and the purification buffer was added again to make the exchanging ratio greater than 1000 times. Thus, the non-conjugated linker-drug compound and other low molecular weight reagents were removed to obtain the purified antibody-drug conjugate (SWY2110-VC MMAE ADC, DAR=4.53).

### Step 4. Determination of Drug-Antibody Ratio (DAR) of Antibody-Drug Conjugate:

10 μg SWY2110-Vc MMAE ADC sample was loaded onto the chromatographic column (2.5 μm particle size; 4.6 mm×10 cm; TSKgel Butyl-NPR). The elution mobile phase A was 20 mM PB, pH 7.0, 1.5 M (NH₄)₂SO₄ aqueous solution, and the mobile phase B was 20 mM PB, pH 7.0, 25% isopropanol solution. The flow rate was controlled at 0.8 mL/min, the column temperature was 30° C., the detection wavelength was 280 nm, and the gradient program was set as follows: 0-20 minutes, mobile phase B from 0 to 100%; 20-25 minutes, 100% mobile phase B; and 25-30 minutes, 100% mobile phase A. Being non-conjugated was DARO, being conjugated with 2 small molecules was DAR2, being conjugated with 4 small molecules was DAR4, being conjugated with 6 small molecules was DAR6, and being conjugated with 8 small molecules was DAR8. Therefore, elution was performed in the order of DARO, DAR2, DAR4, DAR6, and DAR8. The DAR value was calculated based on the peak area at 280 nm, where A represents the percentage of each peak area. A result of the DAR value is shown in FIG. 10.

DAR=2×A_{DAR2}+4×A_{DAR4}+6×A_{DAR6}+8×A_{DAR8}

### Biological Examples

### Assay 1: Activity of Antibody-Drug Conjugate in Cells In Vitro

### 1. Assay Object:

The object of this assay was to detect the inhibitory activity of the antibody-drug conjugate ofthe present invention on the in-vitro proliferation of SK-BR-3 (ATCC/HTB-30) tumor cells, human colorectal cancer cells DiFi, human lung cancer gefitinb resistant cell PC9-GR, or human lung cancer mutant cell PC-9 (Dell9-T790M-C797). The cells were treated in vitro with compounds of different concentrations. After culture, resazurin was added to read the fluorescence values of ex550 nm/em610 nm, and the data were fitted with four parameters to obtain the IC₅₀ values, thereby calculating the biological activities of the compound.

### 2. Assay Materials and Equipments:

### Materials

| Product Name | Manufacturer/Item Number |
|---|---|
| SK-BR-3 cell line | ATCC/HTB-30 |
| DiFi cell line | Shanghai Jinmante Biotechnology |
| | Co., Ltd |
| PC9-GR cell line | Nanjing Cobioer Biosciences |
| | Co., Ltd |
| MTT Assay Kit | Abcam |
| DMEM, high glucose | Hyclone/SH30022.01 |
| 0.25% Trypsin-EDTA | Gibco/25200-072 |
| fetal bovine serum (FBS) | Gibco/16000-044 |
| 100 × bispecific antibody | Gibco/15240-062 |
| black-walled, transparent- | Corning/3603 |
| bottom tissue culture plate | |
| resazurin sodium | Sigma Aldrich/199303-25G |

### Equipments

| Name | Model | Manufacturer |
|---|---|---|
| Biosafety cabinet | 1300 series | Thermo Fisher Scientific |
| | A2 model | |
| CO₂ incubator | 3111 model | Thermo Fisher Scientific |
| Inverted | CKX31 | Olympus |
| microscope | | |
| Microplate reader | Infinite M200 | Tecan Company |
| Micro-oscillator | MM-I model | Shanghai Yarong Biochemistry |
| | | Instrument Factory |

### 3. Assay Operation Procedure 1:

3.1 Culture medium: DMEM, 10% FBS, lxbispecific antibody
3.2 Cell culturing: Frozen SK-BR-3/MDA-MB-468 cells were taken out of liquid nitrogen and revived in a 75 cm² culture vessel for culturing. The cells were grown until the confluency of cells reached >75%, and had undergone at least 3 passages.
3.2.1 If cell passage was not performed, the culture medium should be replaced every 3-4 days.
3.2.2 If the cell expansion was needed, the cells were passaged and inoculated into a larger culture vessel, ensuring that the confluency of cells reached >75% before use in assays.
3.3 Cell Collection: SK-BR-3/MDA-MB-468 cells were collected when the culture vessel was nearly full.
3.3.1 The culture medium was discarded, and the cells were washed with PBS to remove dead cells and residual culture medium.
3.3.2 2-3 mL of 0.25% Trypsin-EDTA was added, the culture vessel was gently shaken, and then incubated at 37° C. for 2-3 minutes to digest the cells.
3.3.3 5 mL of the culture medium was immediately added to the culture vessel and gently pipetted up and down to spread the cells.
3.3.4 The cell suspension was transferred to a sterile centrifuge tube and centrifuged at 200×g for 3 minutes.
3.3.5 The culture medium in the tube was discarded, and 5-10 mL of the fresh culture medium was added to resuspend the cells.
3.4 Cell density determination: The cells were counted with a cell counting plate under a microscope.
3.5 Assay plate inoculation
3.5.1 Cells were diluted to 1×10⁵ cells/mL with the culture medium and seeded at 100 μL/well in the assay plate (except rows A and H).
3.5.2 120 μL of the culture medium was added to each well in rows A and H as a blank.
3.5.3 The incubation was performed at 37° C. with 5% CO₂ for 4-6 hrs to allow the cells to adhere to the wall.
3.6 Preparation of diluted compound samples:

The compound was diluted to an initial concentration of 18 μg/ml, and then further diluted by a factor of 3 to obtain a total of 11 gradient solutions. The 12th column was left as a blank control.
3.7 Drug treatment
3.7.1 20 μL of drug diluent from each of the 1st to 11th columns of the dilution plate was taken and added to the corresponding column of the assay plate.
3.7.2 20 μL of fresh culture medium was added to each well in the 12th column and the A and H rows.
3.7.4 The plate was gently shaken on a plate shaker for 10-15 seconds, and then the culture plate was incubated at 37° C. for 3 days.
3.8 Analyses
3.8.1 After incubation, 20 μL of 0.03% resazurin (1×PBS diluted) was added to each well and gently shaken for 10-15 seconds.
3.8.2 After incubating at 37° C. for 3-4 hours, the plate was read using a microplate reader with the following parameters:
Exciting light: 550 nm
Emitting light: 610 nm
Integration time: 50
Shaking: 15 seconds, vortex
Reading: top reading
Number of reading/well: 1
Gain: optimization setting (between 35 and 42)

If reading multiple plates, it was ensured that the gain setting was consistent for all plates.
3.8.3 EXCEL was used to plot the data and fit the IC₅₀ values for the reference standards and samples.
3.8.3.1 Model 201 was used to plot the data points.
3.8.3.2 For Fit parameters, the following commands were used:
a. A: Pre-fitted
b. B: Pre-fitted
c. C: Pre-fitted
d. D: Pre-fitted
e. No constraints for all
3.8.3.3 The output parameter C represented the IC₅₀ in unit of ng/mL.

The results are shown in Table 2.

### Assay Operation Procedure 2:

This assay verified the inhibitory effects of SWY2110 monoclonal antibody (SWY2110 mAb), SWY2110-DXD ADC (DAR=6.93), and SWY2110-JSSW-001 ADC (DAR=7.12) on the proliferation of two cell lines, DiFi (human colorectal cancer cells) and PC9-GR (gefitinib-resistant human lung cancer cells). The specific concentration settings were as follows:

DiFi: SWY2110 mAb, SWY2110-DXD ADC (DAR=6.93), and SWY2110-JSSW-001 ADC (DAR=7.12) started at a concentration of 3000 ng/ml, with a 3× serial dilution of the drug, resulting in 10 concentrations: 3000, 1000, 333.33, 111.11, 37.04, 12.35, 4.12, 1.37, 0.46, 0.15 ng/ml, respectively. The drug exposure lasted for 144 hours.

PC9-GR: SWY2110 mAb, SWY2110-DXD ADC (DAR=6.93), SWY2110-JSSW-001 ADC (DAR=7.12) started at a concentration of 1000 ng/ml, with a 5× serial dilution of the drug, resulting in 8 concentrations: 1000, 200, 40, 8, 1.6, 0.32, 0.64, 0.013 ng/ml, respectively. The drug exposure lasted for 144 hours.

Cells in logarithmic growth phase were seeded in a 96-well plate (100 μL/well) at a certain number. After the adherent cells had adhered for 24 hours, 100 μL of culture medium containing different concentrations of SWY2110 mAb, SWY2110-DXD ADC (DAR=6.93), or SWY2110-JSSW-001 ADC (DAR=7.12) was added to each well. Three replicate wells were set for each drug concentration, along with corresponding blank wells (only containing culture medium) and normal wells (drug concentration of 0). After 144 hours of drug exposure, a MTT working solution (5 mg/mL) was added to each well (20 μL per well). The plates were incubated at 37° C. for 4 hours, and then the supernatant was discarded. DMSO (analytical grade) was added (150 μL per well), and the plates were shaken on a microplate shaker to mix thoroughly. The plates were then wiped clean, and the optical density (OD) was measured at 550 nm using the microplate reader.

The inhibition rate of the cell growth was calculated with the following equation:

Inhibition Rate(%)=(OD Value_{normal well}−OD Value_{drug well})/(OD Value_{normal well}−OD Value_{blank well})×100%

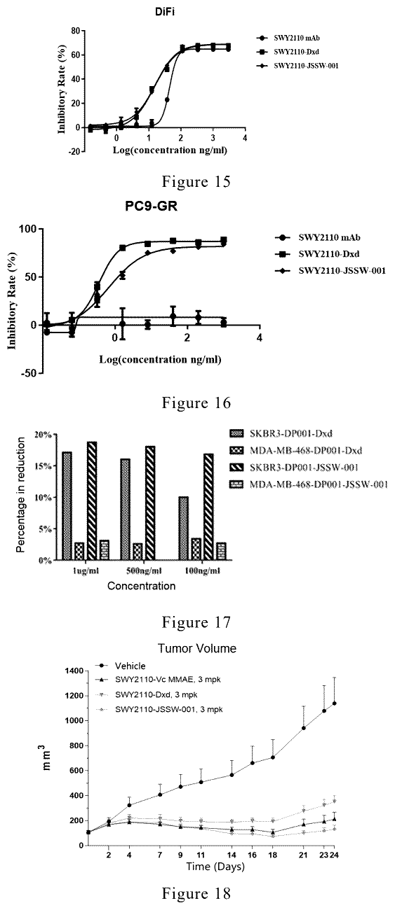

Based on the inhibition rates at different concentrations, the half-maximal inhibitory concentration (IC₅₀) of the drug was calculated using SPSS 19.0. The results are shown in Table 3, and FIGS. 15 and 16.

### Assay Data:

TABLE 2
IC₅₀ value of DP001-ADC against SK-BR-3 cells

| Compound No. | IC₅₀ value (ng/mL) |
|---|---|
| DP001-DXD ADC (DAR = 7.0) | 7.2481 |
| DP001-JSSW-001 ADC (DAR = 7.32) | 5.1824 |

It can be seen from the data recorded in Table 2 that the antibody-drug DP001-JSSW-001 ADC (DAR=7.12) of the present invention had a lower IC₅₀ value against SK-BR-3 cells than DP001-DXD ADC (DAR=6.93), showing better anti-cell proliferation effect.

TABLE 3
IC₅₀ value of SWY2110-ADC against two cell lines (ng/mL)

| | | SWY2110- | SWY2110- |
|---|---|---|---|
| | SWY2110 | DXD ADC | JSSW-001 ADC |
| Cell | mAb | (DAR = 6.93) | (DAR = 7.12) |
| DiFi | 43.94 | 14.85 | 15.84 |
| PC9-GR | — | 1.00 | 1.12 |

wherein “--” represents not showing anti-cell proliferation effect.

It can be seen from the data in Table 3 that the antibody-drug conjugates SWY2110-JSSW-001 ADC (DAR=7.12) and SWY2110-DXD ADC (DAR=6.93) of the present invention had similar IC₅₀ values for EGFR overexpressing cell line DiFi and gefitinib-resistant cell line PC9-GR, and showed better anti-cell proliferation effect compared to SWY2110 mAb.
Assay 2: Efficiency assay of antibody-drug conjugates on HER2-expressing human breast cancer JIMT-1 xenograft tumor in NU/NU mice
1. Assay animals

NU/NU mice, 5-6 weeks old, 24 mice
2. Assay object

To investigate the antitumor effect of antibody-drug conjugates in vivo using a HER2-expressing human breast cancer JIMT-1 cell xenograft tumor model in NU/NU mice.
3. Drug dosage and grouping

TABLE 4
Table of animal grouping and dosage

| | Animal | Dosage | Administration |
|---|---|---|---|
| Group | Number | (mg/kg) | Route/Frequency |
| Vehicle | 6 | 0 | i.v./once |
| DP001-DXD ADC | 6 | 3 | i.v./once |
| (DAR = 4.30) | | | |
| DP001-JSSW-001 ADC | 6 | 3 | i.v./once |
| (DAR = 4.0) | | | |
| DP001-ZW-002 ADC | 6 | 3 | i.v./once |
| (DAR = 5.78) | | | |

Note:
i.v.: intravenous injection.

4. Assay method

In this assay, human breast cancer JIMT-1 cells were used to construct a xenograft tumor model in nude mice. When the tumor volume reached about 127 mm³, the animals were evenly divided into 4 groups (d0) according to Table 4 based on tumor volume with 6 animals in each group. Intravenous administration was given once, with an administration volume of 10 mL/kg. The vehicle control group (Vehicle) received a 0.9% sodium chloride injection. Observations were made for 13 days after administration to compare the inhibitory effects of three antibody-drug conjugates in the table on HER2-expressing human breast cancer JIMT-1 xenograft tumors in nude mice.
5. Observation indicator
5.1 Evaluation indicator
(1) Tumor volume: V=½×A×B²
(2) Relative tumor volume:

$$RTV = \frac{{TV}_{nd}}{{TV}_{0 d}}$$

(3) Relative tumor volume proliferation rate:

$$\frac{T}{C} \% = \frac{Drug Group {RTV}_{xnd}}{Vehicle Group {RTV}_{xnd}} \times 100 \%$$

(4) Tumor (growth) inhibition rate:

$$TGI \% = [ 1 - \frac{{TV}_{Xn} - {TV}_{X 0}}{{TV}_{Mn} - {TV}_{M 0}} ] \times 100 \%$$

Note: V: Tumor volume
A: tumor length
B: tumor width
RTV: relative tumor volume
TV_{nd}: tumor volume on day n
TV_{0d}: tumor volume on day 0
RTV_{xnd}: average relative tumor volume on day n
TV_{Xn}: average tumor volume of drug group on day n
TV_{X0}: average tumor volume of drug group on day 0
TV_{Mn}: average tumor volume of vehicle group on day n
TV_{M0}: average tumor volume of vehicle group on day 0
6 Result
6.1 Tumor volume

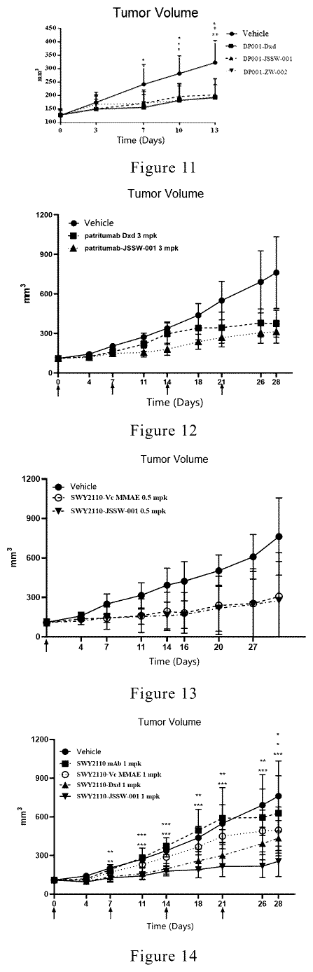

At the end of the assay, compared with the vehicle group, DP001-DXD ADC (DAR=4.3) (P<0.05), DP001-JSSW-001 ADC (DAR=4.0) (P<0.01) and DP001-ZW-002 ADC (DAR=5.78) (P<0.05) all could significantly inhibit the volume of transplantation tumor (see FIG. 11 and Table 5 for details).

TABLE 5
Tumor parameters of each group 13 days after
administration of antibody-drug conjugates

| | | | Tumor | TGI |
|---|---|---|---|---|
| | Animal | Dosage | Volume | (%) |
| Group | Number | (mg/kg) | (mm³) | Day 13 |
| Vehicle | 6 | 0 | 322.7 ± 82.2 | — |
| DP001-DXD ADC | 6 | 3 | 192.7 ± 70.2* | 67.3 |
| (DAR = 4.30) | | | | |
| DP001-JSSW-001 ADC | 6 | 3 | 202.9 ± 38.1** | 61.4 |
| (DAR = 4.0) | | | | |
| DP001-ZW-002 ADC | 6 | 3 | 194.8 ± 68.2* | 65.3 |
| (DAR = 5.78) | | | | |

*compared with the vehicle group P < 0.05,
**compared with the vehicle group P < 0.01

Assay 3: Efficacy assay of antibody-drug conjugate on human lung cancer PC9-GR (gefitinib-resistant cell) xenograft tumor in NU/NU mice
1. Assay animals

NU/NU mice, 5-6 weeks old, 16 mice
2. Assay object

To investigate the anti-tumor effect of two patritumab-ADC drugs in vivo using a human lung cancer PC-9/GR cell (gefitinib-resistant cell) xenograft tumor model in NU/NU mice.
3. Drug dosage and grouping

TABLE 6
Table of animal grouping and dosage

| | Animal | Dosage | Administration |
|---|---|---|---|
| Group | Number | (mg/kg) | Route/Frequency |
| Vehicle | 6 | 0 | i.v./qw*4w |
| patritumab-DXD ADC | 5 | 3 | i.v./qw*4w |
| (DAR = 7.3) | | | |
| patritumab-JSSW-001 ADC | 5 | 3 | i.v./qw*4w |
| (DAR = 7.36) | | | |

Note:
i.v.: intravenous injection, qw*4w: administration once weekly for 4 weeks.

4. Assay method

In this assay, human lung cancer PC9-GR cells (gefitinib-resistant cells) were used to construct a xenograft tumor model in nude mice. When the tumor volume reached approximately 110 mm³, the animals were divided into 3 groups (d0) based on tumor volume according to Table 6, with 6 animals in vehicle group, each 5 animals in patritumab-DXD ADC (DAR=7.3) group and patritumab-JSSW-001 ADC (DAR=7.36) group. Intravenous administration was given once weekly for 4 weeks, with an administration volume of 10 mL/kg. The vehicle control group (Vehicle) received a 0.9% sodium chloride injection. The assay was terminated on day 28 after the administration. The inhibitory effects of patritumab-DXD ADC (DAR=7.3) and patritumab-JSSW-001 ADC (DAR=7.36) on human lung cancer PC9-GR (Gefitinib-resistant cell) xenograft tumor in nude mice were compared.
5. Observation indicator
5.1 Evaluation indicator

See the section of Assay 2: 5.1 Evaluation indicator
6 Result
6.1 Tumor volume

At the end of the assay, the patritumab-JSSW-001 ADC (DAR=7.36) group (P<0.001) exhibited the superior effect of inhibiting the volume of xenograft tumor compared to the patritumab-DXD ADC (DAR=7.3) (P<0.01) group. For details, see FIG. 12 and Table 7.

TABLE 7
Tumor parameters of each group 28 days after administration of patritumab-ADC

| | | | | Tumor | TGI |
|---|---|---|---|---|---|
| | Animal | Dosage | Administration | Volume | (%) |
| Group | Number | (mg/kg) | Route/Frequency | (mm³) | Day 28 |
| Vehicle | 6 | 0 | i.v./qw*4w | 761.4 ± 272.6 | — |
| patritumab- | 5 | 3 | i.v./qw*4w | 373.4 ± 102.8** | 59.5 |
| DXD ADC | | | | | |
| (DAR = 7.3) | | | | | |
| patritumab- | 5 | 3 | i.v./qw*4w | 312.7 ± 86.7*** | 68.9 |
| JSSW-001 ADC | | | | | |
| (DAR = 7.36) | | | | | |

**compared with the vehicle group P < 0.01,
***compared with the vehicle group P < 0.001

Assay 4: Efficacy Assay of SWY2110-ADC on human colorectal cancer DiFi xenograft tumor in NU/NU mice
1. Assay animals

NU/NU mice, 5-6 weeks old, 14 mice
2. Assay object

To investigate the anti-tumor effect of two SWY2110-ADC drugs in vivo using a human colorectal cancer DiFi cell xenograft tumor model in NU/NU mice.
3. Drug dosage and grouping

TABLE 8
Table of animal grouping and dosage

| | Animal | Dosage | Administration |
|---|---|---|---|
| Group | Number | (mg/kg) | Route/Frequency |
| Vehicle | 5 | 0 | i.v./once |
| SWY2110-Vc MMAE ADC | 4 | 0.5 | i.v./once |
| (DAR = 4.53) | | | |
| SWY2110-JSSW-001 ADC | 5 | 0.5 | i.v./once |
| (DAR = 7.12) | | | |

Note:
i.v.: intravenous injection.

4. Assay method

In this assay, human colorectal cancer DiFi cells were used to construct a xenograft tumor model in nude mice. When the tumor volume reached approximately 110 mm³, the animals were evenly divided into 3 groups (dO) based on tumor volume according to Table 8, with each 5 animals in vehicle group and SWY2110-JSSW-001 ADC (DAR=7.12) group and 4 animals in SWY2110-Vc MMAE ADC (DAR=4.53) group. Intravenous administration was given once with an administration volume of 10 mL/kg. The vehicle control group (Vehicle) received a 0.9% sodium chloride injection. Observation was made for 27 days after the administration. The inhibitory effects of two SWY2110-ADC drugs in the table on human colorectal cancer DiFi xenograft tumor in nude mice were compared.
5. Observation indicator
5.1 Evaluation indicator

See the section of Assay 2: 5.1 Evaluation indicator
6 Result
6.1 Tumor volume

At the end of the assay, compared with the vehicle group, both SWY2110-Vc MMAE ADC (DAR=4.53) (P<0.05) and SWY2110-JSSW-001 ADC (DAR=7.12) (P<0.05) could significantly inhibit the volume of the xenograft tumor. The tumor inhibition rate on day 27 of the SWY2110-JSSW-001 ADC (DAR=7.12) group reached as high as 74.6%, followed by the SWY2110-Vc MMAE ADC (DAR=4.53) group with a tumor inhibition rate of 69.7%. Since the toxicity of the JSSW-001 small molecule was lower than that of Vc MMAE, it was suggested that SWY2110-JSSW-001 ADC (DAR=7.12) could achieve a more efficient and less toxic therapeutic effect. For details, see FIG. 13 and Table 9.

TABLE 9
Tumor parameters of each group 27 days
after administration of SWY2110-ADC

| | | | Tumor | TGI |
|---|---|---|---|---|
| | Animal | Dosage | Volume | (%) |
| Group | Number | (mg/kg) | (mm³) | Day 27 |
| Vehicle | 5 | 0 | 763.1 ± 293.8 | — |
| SWY2110-Vc MMAE | 4 | 0.5 | 306.5 ± 332.4* | 69.7 |
| ADC (DAR = 4.53) | | | | |
| SWY2110-JSSW-001 | 5 | 0.5 | 275.5 ± 297.5* | 74.6 |
| ADC (DAR = 7.12) | | | | |

*compared with the vehicle group P < 0.05

Assay 5 Efficacy assay of SWY2110-ADC on human lung cancer PC9-GR (gefitinib-resistant cell) xenograft tumor in NU/NU mice
1. Assay animals

NU/NU mice, 5-6 weeks old, 30 mice
2. Assay object

To investigate the anti-tumor effect of SWY2110 monoclonal antibody and three SWY2110-ADC drugs in vivo using a human lung cancer PC-9-GR cell (gefitinib-resistant cell) xenograft tumor model in NU/NU mice.
3. Drug dosage and grouping

TABLE 10
Table of animal grouping and dosage

| | Animal | Dosage | Administration |
|---|---|---|---|
| Group | Number | (mg/kg) | Route/Frequency |
| Vehicle | 6 | 0 | i.v./qw*4w |
| SWY2110 mAb | 6 | 1 | i.v./qw*4w |
| SWY2110-Vc MMAE ADC | 6 | 1 | i.v./qw*4w |
| (DAR = 4.53) | | | |
| SWY2110-DXD ADC | 6 | 1 | i.v./qw*4w |
| (DAR = 6.93) | | | |
| SWY2110-JSSW-001 ADC | 6 | 1 | i.v./qw*4w |
| (DAR = 7.12) | | | |

Note:
i.v.: intravenous injection, qw*4w: administration once weekly for 4 weeks.

4. Assay method

In this assay, human lung cancer PC9-GR cells (gefitinib-resistant cells) were used to construct a xenograft tumor model in nude mice. When the tumor volume reached approximately 110 mm³, the animals were evenly divided into 5 groups (d0) based on tumor volume according to Table 10, with 6 animals in each group. Intravenous administration was given once weekly for 4 weeks, with an administration volume of 10 mL/kg. The vehicle control group (Vehicle) received a 0.9% sodium chloride injection. The assay was terminated on day 28 after the administration. The inhibitory effects of SWY2110 monoclonal antibody and three SWY2110-ADC drugs on human lung cancer PC9-GR (Gefitinib-resistant cell) xenograft tumor in nude mice were compared.
5. Observation indicator
5.1 Evaluation indicator

See the section of Assay 2: 5.1 Evaluation indicator
6 Result
6.1 Tumor volume

At the end of the assay, compared with the vehicle group, SWY2110-Vc MMAE ADC (DAR=4.53) (P<0.05), SWY2110-DXD ADC (DAR=6.93) (P<0.05), and SWY2110-JSSW-001 ADC (DAR=7.12) (P<0.001) all exhibited inhibitory effects on the volume of the xenograft tumor. The antibody SWY2110 showed poor tumor inhibitory effect, with a tumor inhibition rate of only 20.3%. The SWY2110-JSSW-001 ADC (DAR=7.12) group had the most significant inhibitory effect on the xenograft tumor volume of PC9-GR, with a tumor inhibition rate as high as 77.7%. The tumor inhibition rates of SWY2110-Vc MMAE ADC (DAR=4.53) and SWY2110-DXD ADC (DAR=6.93) were 40.4% and 50.2%, respectively. SWY2110-JSSW-001 ADC (DAR=7.12) showed 154% of the tumor inhibitory effect of SWY2110-DXD ADC (DAR=6.93) on the PCR-GR, and SWY2110-JSSW-001 ADC (DAR=7.12) showed 192% of the tumor inhibitory effect of SWY2110-Vc MMAE ADC (DAR=4.53) on PCR-GR, suggesting that SWY2110-JSSW-001 ADC (DAR=7.12) still had a significant inhibitory effect on gefitinib-resistant tumor cells. For details, see FIG. 14 and Table 11.

TABLE 11
Tumor parameters of each group 28 days after administration of SWY2110-ADC

| | | | | Tumor | TGI |
|---|---|---|---|---|---|
| | Animal | Dosage | Administration | Volume | (%) |
| Group | Number | (mg/kg) | Route/Frequency | (mm³) | Day 28 |
| Vehicle | 6 | 0 | i.v./qw*4w | 761.4 ± 272.6 | — |
| SWY2110 mAb | 6 | 1 | i.v./qw*4w | 628.9 ± 290.0 | 20.3 |
| SWY2110-Vc | 6 | 1 | i.v./qw*4w | 498.0 ± 179.1* | 40.4 |
| MMAE ADC | | | | | |
| (DAR = 4.53) | | | | | |
| SWY2110- | 6 | 1 | i.v./qw*4w | 434.6 ± 136.3* | 50.2 |
| DXD ADC | | | | | |
| (DAR = 6.93) | | | | | |
| SWY2110- | 6 | 1 | i.v./qw*4w | 255.1 ± 117.7*** | 77.7 |
| JSSW-001 ADC | | | | | |
| (DAR = 7.12) | | | | | |

*compared with the vehicle group P < 0.05,
***compared with the vehicle group P < 0.001

### Assay 6: In Vitro Plasma Stability of Antibody-Drug Conjugates

DP001-DXD ADC (DAR=7.0) and DP01-JSSW-001 ADC (DAR=7.32) were incubated with 0.5 BSA-PBS and ICR mouse plasma, respectively, at concentrations of DP001-DXD ADC (DAR=7.0) (91.7 μg/mL) and DP001-JSSW-001 ADC (DAR=7.32) (80 μg/mL). Samples were collected after incubation for Oh, 24 h, 48 h, 72 h, 96 h, 120 h, 144 h, 168 h, 336 h and 504 h, respectively, under sterile conditions at 37° C. The concentration of dropped molecules in the plasma samples was detected using LC-MS/MS. The results are as follows:

TABLE 12
In vitro plasma stability

| | DP001-DXD ADC (DAR = 7.0) | | DP001-JSSW-001 ADC (DAR = 7.32) | |
|---|---|---|---|---|
| Time | 0.5% BSA-PBS | ICR | 0.5% BSA-PBS | ICR |
| (h) | Dropping rate (%) | Dropping rate (%) | Dropping rate (%) | Dropping rate (%) |
| 0 | 0.02 | 0.02 | 0.03 | 0.01 |
| 24 | — | — | 0.13 | 0.15 |
| 48 | 0.13 | 0.61 | 0.16 | 1.02 |
| 72 | 0.13 | 0.65 | 0.18 | 1.13 |
| 96 | 0.13 | 0.97 | 0.20 | 0.77 |
| 120 | 0.16 | 1.18 | — | 0.82 |
| 144 | 0.16 | 1.29 | 0.19 | 1.18 |
| 168 | 0.14 | 1.43 | 0.20 | 1.36 |
| 336 | 0.41 | 2.50 | 0.27 | 1.54 |
| 504 | 3.90 | 4.07 | 1.09 | 2.75 |

Note:
1. Calculation of the dropping rate: according to the DAR value, the mass ratio of DXD/ADC was calculated to determine DP001-DXD ADC (DAR = 7.0) (91.7 μg/mL) and DP001-JSSW-001 ADC (DAR = 7.32) (80 μg/mL). Dropping rate (%) = Ct/Ctotal*100%, wherein Ctotal represents the total small molecule concentration, and Ct represents the free small molecule concentration at each time point.
“—” indicates no sampling.

The results showed that ADCs generated free small molecules in both 0.5% BSA-PBS and ICR mouse plasma after incubation at 37° C. for Oh, 24 h, 48 h, 72 h, 96 h, 120 h, 144 h, 168 h, 336 h and 504 h. After 504 hours of incubation, the dropping rates in 0.5% BSA-PBS were DP001-DXD ADC (DAR=7.0) (3.90%) and DP001-JSSW-001 ADC (DAR=7.32) (1.09%) respectively, and in ICR mouse plasma, the dropping rates were DP001-DXD ADC (DAR=7.0) (4.07%) and DP001-JSSW-001 ADC (DAR=7.32) (2.75%) respectively. In 0.5% BSA-PBS, the dropping rates of small molecules from the antibodies were all less than 4.0%: DP001-DXD ADC (DAR=7.0)>DP001-JSSW-001 ADC (DAR=7.32), indicating that DP001-JSSW-001 ADC (DAR=7.32) was more stable in 0.5% BSA-PBS. In ICR mouse plasma, the dropping rates of small molecules from the antibodies were all less than 5.0%: DP001-DXD ADC (DAR=7.0)>DP001-JSSW-001 ADC (DAR=7.32), indicating that DP001-JSSW-001 ADC (DAR=7.32) was more stable in ICR mouse plasma than DP001-DXD ADC (DAR=7.0).

### Assay 7: Study on In Vitro Bystander Effect of Antibody-Drug

For the study on the bystander effect, positive cell line SK-BR-3 and negative control cell MDA-MB-468 were co-cultured with DP001-DXD ADC (DAR=7.0) and DP001-JSSW-001 ADC (DAR=7.32), respectively. The released small molecule payloads could cause apoptosis of co-cultured negative cells in the periphery, known as the bystander effect. In this study, reporter gene cells HEK293-Luc were co-cultured with positive cell line SK-BR-3 and negative control cell MDA-MB-468 for four days. The effect of released small molecule payloads on the growth of HEK293-Luc was determined by adding a chemiluminescent substrate. The study results showed that DP001-DXD ADC (DAR=7.0) and DP001-JSSW-001 ADC (DAR=7.32) could bind to HER2 receptors on the surface of tumor cells, enter the cells through endocytosis and release small molecule payloads. Meanwhile, DP001-JSSW-001 ADC (DAR=7.32) showed a stronger inhibitory effect on the proliferation of bystander cells.
2. Assay preparation
2.1 Assay equipments:

| Apparatus and equipment | Manufacturer | Model |
|---|---|---|
| CO₂ incubator | Thermo Fisher | 3111 model |
| Biosafety cabinet | ThermoFisher | 1300 Series A2 6 inches |
| Desktop high speed | Thermo Fisher | SORVALL Stratos |
| freezing centrifuge | | |
| Inverted microscope | Olympus | CKX31 inverted microscope |
| Pipettor | Eppendorf | Research Plus |
| Microplate reader | Tecan | M200 |

2.2 Assay materials:

| Name | Manufacturer | Item Number |
|---|---|---|
| 96-well transparent flat- | Corning | 3599 |
| bottom culture plate | | |
| cell culture plate | Corning | 3917 |
| 50 mL centrifuge tube | Corning | 430828 |
| 15 mL centrifuge tube | Corning | 430790 |
| T75 cell culture flask | Corning | 430641 |
| T25 cell culture flask | Corning | 430639 |

2.3 Assay reagents.

| Name | Manufacturer | Item Number |
|---|---|---|
| DPBS | BBI | E607009-0500 |
| Fetal Bovine Serum | Gibco | 10099-141C |
| DMEM culture medium | HyClone | SH30243.01 |
| 0.25% Trypsin-EDTA | Gibco | 25200-072 |
| Bright-GloTM Luciferase assay system | Promega | E2620 |

2.4 Cell information

| Name | Manufacturer | Culture condition |
|---|---|---|
| SK-BR-3 | ATCC | DMEM culture medium + 10% |
| MDA-MB-468 | Nanjing Cobioer | DMEM culture medium + 10% |
| HEK293-Luc | self-made in the | DMEM culture medium + 10% |
| | laboratory | FBS + 400 μg/ml G418 + |
| | | 100 μg/ml HygromycinB |

3. Assay method
3.1 Reagent preparation
3.1.1 Preparation of cell recovery medium

DMEM complete medium: Adding to a DMEM basic culture medium so that the final system contained 10% of Fetal Bovine Serium and 1% of Penicilin/Streptomycin, fully mixing uniformly, and storing at 2-8° C.
3.1.2 preparation of cell culture medium (containing a screening agent)

Adding to a DMEM basic culture medium so that the final system contained 10% of Fetal Bovine Serium and 1% of Penicilin/Streptomycin and adding 200 μg/mL of G418, fully mixing uniformly, and storing at 2-8° C.
3.2 Cell preparation
3.2.1 Cell recovery

A cell cryopreservation tube was placed in a water bath at 37° C. and shaken quickly until it was completely thawed, then transferred to an ultra-clean workbench or a biosafety cabinet. The cell suspension was diluted with a cell recovery medium. The mixture was centrifuged, and the centrifugation supernatant was discarded. Cells were resuspended by using the cell recovery medium, and the cell density and viability were measured. The cell density was adjusted with the cell culture medium. The cell suspension was uniformly mixed, transferred to a cell culture flask. After the microscopic examining, it was transferred to an incubator for culturing at 37° C. with 5% CO₂.
3.2.2 cell passages

The cell culture flask was removed from the CO₂ incubator and transferred to the ultra-clean workbench or biosafety cabinet. After the cells were washed with PBS, digested with trypsin, and the digestion was terminated with cell growth medium, the cell suspension was collected, and cell density and viability were measured. An appropriate amount of the cell suspension was added to a suitable amount of the fresh medium (with or without a screening agent), and the cells were passaged and expanded according to their growth characteristics and experimental needs. After passaging, the cells were placed in an incubator for continued culture.
3.3 Bystander effect test procedure
3.3.1 When the cell confluence reached 90%, SK-BR-3, MDA-MB-468, and HEK293-Luc cells were digested with trypsin and collected separately, and the cells were resuspended in their respective media.
3.3.2 The resuspended target cells were gently pipetted several times to form a single-cell suspension. Cell viability and cell count were identified using the trypan blue staining method, and the cell density was adjusted to 2.0×105 cells/mL.
3.3.3 The density-adjusted SK-BR-3 and MDA-MB-468 cells were evenly mixed with HEK293-Luc cell suspension at a 1:1 ratio. Fifty microliters per well were added to a 96-well transparent flat-bottom culture plate. The positive cell group was a mixture of SK-BR-3 and HEK293-Luc cells, while the negative cell group was a mixture of MDA-MB-468 and HEK293-Luc cells.
3.3.4 DP001-DXD ADC (DAR=7.0) and DP001-JSSW-001 ADC (DAR=7.32) groups (drug administration groups) were provided at three concentrations with final concentrations of 100 ng/ml, 500 ng/ml, and 1000 ng/ml. Fifty microliters per well were added to the cell-inoculated 96-well transparent flat-bottom culture plate and placed in a 37° C. CO₂ incubator for 4 days.
3.3.5 After incubation, the plate was removed from the incubator, and the temperature was allowed to equilibrate to room temperature. Bright-Glo™ Luciferase Assay System was added to the plate at 100 μL per well, and chemiluminescence was read after 5-15 minutes in the dark.
4. Test result: DP001-DXD ADC (DAR=7.0) and DP001-JSSW-001 ADC (DAR=7.32) could bind to HER2 receptors on the surface of tumor cells, enter the cells through endocytosis and release small molecule payloads. Meanwhile, DP001-JSSW-001 ADC (DAR=7.32) showed a stronger inhibitory effect on the proliferation of bystander cells than DP001-DXD ADC (DAR=7.0). See Table 13 and FIG. 17 for the test result.

TABLE 13
Inhibition rate of the antibody-drug conjugates
of the present invention on fluorescent cells

| | SKBR3 + | | | MDA-MB-468 + |
|---|---|---|---|---|
| | HEK293- | MDA-MB-468 + | SKBR3 + HEK293- | HEK293- |
| | GLP1/ | HEK293- | GLP1/ | GLP1/DP001- |
| | DP001-DXD | GLP1/DP001- | DP001-JSSW- | JSSW-001 |
| | ADC((DAR = | DXD ADC | 001 ADC | ADC |
| Concentration/ | 7.0) | (DAR = 7.0) | (DAR = 7.32) | (DAR = 7.32) |
| ng/ml | Inhibition rate | | | |
| 1000 | 17.1% | 2.7% | 18.7% | 3.1% |
| 500 | 16.4% | 2.6% | 18.1% | −0.4% |
| 100 | 10.0% | 3.4% | 16.8% | 2.7% |

### Assay 8: Study on the Stability of Linker-Drug Compounds

Different linker-drug compounds such as Deruxtecan and JSSW-001 were studied under high temperature (40° C., 60° C.), high humidity (25° C./75% RH and 25° C./92.5% RH), illumination (illuminance 4500Lux, near-UV energy 90 μw/cm²), or 0.5% BSA-PBS conditions. Samples were collected at different times after exposure to these conditions, and the concentration changes of the linker-drug compounds in the samples were detected using the LC-MS/MS method. The test results showed that the compound JSSW-001 had better stability.

### Assay 9: Efficacy Assay of SWY2110-ADC on Human Lung Cancer PC9-AR (PC9-Dell9/T790M/C797S, Osimertinib-Resistant Cell) Xenograft Tumor

1. Assay animals

Balb/c nude mice, 5-6 weeks old, 24 mice
2. Assay object

To investigate the anti-tumor effect of three SWY2110-ADC drugs in vivo using a human lung cancer PC9-Del19/T790M/C797S cells (osimertinib-resistant cell, hereinafter referred to as PC9-AR) xenograft tumor model in NU/NU mice.
3. Drug dosage and grouping

TABLE 14
Table of animal grouping and dosage

| | Animal | Dosage | Administration |
|---|---|---|---|
| Group | Number | (mg/kg) | Route/Frequency |
| Vehicle | 6 | 0 | i.v./once |
| SWY2110-Vc MMAE ADC | 6 | 3 | i.v./once |
| (DAR = 4.53) | | | |
| SWY2110-DXD ADC | 6 | 3 | i.v./once |
| (DAR = 6.93) | | | |
| SWY2110-JSSW-001 ADC | 6 | 3 | i.v./once |
| (DAR = 7.12) | | | |

Note:
i.v.: intravenous injection, i.v./once: administration once.

4. Assay method

In this assay, human lung cancer PC9-Del19/T790M/C797S cells (osimertinib-resistant cell) were used to construct a xenograft tumor model in nude mice. When the tumor volume reached approximately 110 mm³, the animals were evenly divided into 4 groups (d0) based on tumor volume according to Table 14, with 6 animals in each group. Intravenous administration was given once, with an administration volume of 10 mL/kg. The vehicle control group (Vehicle) received a 0.9% sodium chloride injection. The assay was terminated on day 24 after the administration. The inhibitory effects of three SWY2110-ADC drugs on human lung cancer PC9-Del19/T790M/C797S (osimertinib-resistant cell) xenograft tumor in nude mice were compared.
5. Observation indicator
5.1 Evaluation indicator

See the section of Assay 2: 5.1 Evaluation indicator
6 Result
6.1 Tumor volume

At the end of the assay, compared with the vehicle group, SWY2110-Vc MMAE ADC (DAR=4.53) (P<0.001), SWY2110-DXD ADC (DAR=6.93) (P<0.001) and SWY2110-JSSW-001 ADC (DAR=7.12) (P<0.001) all exhibited inhibitory effects on the volume of the xenograft tumor. The SWY2110-JSSW-001 ADC (DAR=7.12) group demonstrated the most significant inhibitory effect on the PC9-AR xenograft tumor volume, achieving a tumor inhibition rate of as high as 97.5%. The tumor inhibition rates of SWY2110-Vc MMAE ADC (DAR=4.53) and SWY2110-DXD ADC (DAR=6.93) were 89.7% and 76.2% respectively. SWY2110-JSSW-001 ADC (DAR=7.12) showed 128% of the tumor inhibitory effect of SWY2110-DXD ADC (DAR=6.93), and SWY2110-JSSW-001 ADC (DAR=7.12) showed 109% of the tumor inhibitory effect of SWY2110-Vc MMAE ADC (DAR=4.53), suggesting that SWY2110-JSSW-001 ADC (DAR=7.12) still had a significant inhibitory effect on osimertinib-resistant tumor cells. For details, see FIG. 18 and Table 15.

TABLE 15
Tumor parameters of each group 24 days after administration of SWY2110-ADC

| | | | | Tumor | TGI |
|---|---|---|---|---|---|
| | Animal | Dosage | Administration | Volume | (%) |
| Group | Number | (mg/kg) | Route/Frequency | (mm³) | Day 24 |
| Vehicle | 6 | 0 | i.v./once | 1138.3 | — |
| SWY2110-Vc | 6 | 3 | i.v./once | 212.3*** | 89.7 |
| MMAE ADC | | | | | |
| (DAR = 4.53) | | | | | |
| SWY2110- | 6 | 3 | i.v./once | 352.0*** | 76.2 |
| DXD ADC | | | | | |
| (DAR = 6.93) | | | | | |
| SWY2110-JSSW- | 6 | 3 | i.v./once | 131.3*** | 97.5 |
| 001 ADC | | | | | |
| (DAR = 7.12) | | | | | |

***statistical significance relative to the vehicle group p < 0.001

### Assay 10 Efficacy Assay of SWY2110-ADC, SWY2111-ADC, SWY2112-ADC, SWY2113-ADC on Human Lung Cancer NCI-H1975 (Human Lung Cancer Cell) Xenograft Tumor

1. Assay animals

NU/NU mice, 5-6 weeks old, 30 mice
2. Assay object

To investigate the anti-tumor effect of SWY2110-JSSW-001, SWY2111-JSSW-001, SWY2112-JSSW-001, SWY2113-JSSW-001 drugs in vivo using a human lung cancer NCI-H1975 cell (human lung cancer cell) xenograft tumor model in NU/NU mice.
3. Drug dosage and grouping

TABLE 16
Table of animal grouping and dosage

| | Animal | Dosage | Administration |
|---|---|---|---|
| Group | Number | (mg/kg) | Route/Frequency |
| Vehicle | 6 | — | — |
| SWY2110-JSSW- | 6 | 1 | i.v./once |
| 001 ADC | | | |
| (DAR = 7.12) | | | |
| SWY2111-JSSW- | 6 | 1 | i.v./once |
| 001 ADC | | | |
| (DAR = 7.48) | | | |
| SWY2112-JSSW- | 6 | 1 | i.v./once |
| 001 ADC | | | |
| (DAR = 7.51) | | | |
| SWY2113-JSSW- | 6 | 1 | i.v./once |
| 001 ADC | | | |
| (DAR = 7.50) | | | |

Note:
i.v.: intravenous injection,
i.v./once: administration once;

4. Assay method

In this assay, human lung cancer NCI-H1975 cells (human lung cancer cells) were used to construct a xenograft tumor model in nude mice. When the tumor volume reached approximately 110 mm³, the animals were evenly divided into 5 groups (d0) based on tumor volume according to Table 16, with 6 animals in each group. Each dosage group received a single administration at 1 mg/kg. Intravenous administration was given once, with an administration volume of 10 mL/kg. The vehicle control group (Vehicle) received a 0.9% sodium chloride injection. The assay was terminated on day 21 after the administration. The inhibitory effects of SWY2110-JSSW-001, SWY2111-JSSW-001, SWY2112-JSSW-001, SWY2113-JSSW-001 drugs on human lung cancer NCI-H1975 xenograft tumor in nude mice were compared.
5. Observation indicator
5.1 Evaluation indicator

See Assay 2: the part of 5.1 Evaluation indicator
6 Result
6.1 Tumor volume

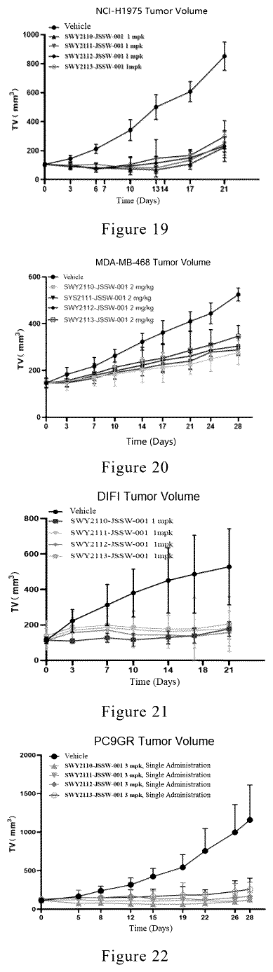

At the end of the assay, compared to the vehicle group, SWY2110-JSSW-001 ADC (DAR=7.12), SWY21 12-JSSW-001 ADC (DAR=7.5 1), SWY2111-JSSW-001 ADC (DAR=7.48), SWY2113-JSSW-001 ADC (DAR=7.50) (P<0.001) all demonstrated significant tumor inhibitory effects (P<0.001), achieving tumor inhibition rates of 84.1%, 83.0%, 80.9% and 74.2% (P<0.001) respectively. These results indicated that all four ADCs had significant inhibitory effects on human lung cancer NCI-H1975 tumor cells. For details, see FIG. 19 and Table 17.

TABLE 17
Tumor parameters of each group 21 days after
administration of SWY2110 to SWY2113-ADCs

| | | | | Tumor | TGI |
|---|---|---|---|---|---|
| | Animal | Dosage | Administration | volume | (%) |
| Group | Number | (mg/kg) | Route/Frequency | (mm³) | Day 21 |
| Vehicle | 6 | 0 | i.v./once | 852.4 ± 97.8 | — |
| SWY2110- | 6 | 1 | i.v./once | 223.3 ± 71.7*** | 84.2 |
| JSSW-001 ADC | | | | | |
| (DAR = 7.12) | | | | | |
| SWY2111- | 6 | 1 | i.v./once | 248.4 ± 80.3*** | 80.9 |
| JSSW-001 ADC | | | | | |
| (DAR = 7.48) | | | | | |
| SWY2112- | 6 | 1 | i.v./once | 231.8 ± 106.5*** | 83.0 |
| JSSW-001 ADC | | | | | |
| (DAR = 7.51) | | | | | |
| SWY2113- | 6 | 1 | i.v./once | 298.4 ± 108.4*** | 74.2 |
| JSSW-001 ADC | | | | | |
| (DAR = 7.50) | | | | | |

***compared with the vehicle group P < 0.001

### Assay 11: Efficacy Assay of SWY2110-ADC, SWY2111-ADC, SWY2112-ADC, SWY2113-ADC on Human Breast Cancer MDA-MB-468 (Human Breast Cancer Cell) Xenograft Tumor

1. Assay animals

NOD-SCID mice, 5-6 weeks old, 28 mice
2. Assay object

To investigate the anti-tumor effect of SWY2110-JSSW-001, SWY2111-JSSW-001, SWY2112-JSSW-001, SWY2113-JSSW-001 drugs in vivo using a human breast cancer MDA-MB-468 cell (human breast cancer cell) xenograft tumor model in NOD-SCID mice.
3. Drug dosage and grouping

TABLE 18
Table of animal grouping and dosage

| | Animal | Dosage | Administration |
|---|---|---|---|
| Group | Number | (mg/kg) | Route/Frequency |
| Vehicle | 8 | 0 | i.v./once |
| SWY2110-JSSW-001 ADC | 5 | 2 | i.v./once |
| (DAR = 7.12) | | | |
| SWY2111-JSSW-001 ADC | 5 | 2 | i.v./once |
| (DAR = 7.48) | | | |
| SWY2112-JSSW-001 ADC | 5 | 2 | i.v./once |
| (DAR = 7.51) | | | |
| SWY2113-JSSW-001 ADC | 5 | 2 | i.v./once |
| (DAR = 7.50) | | | |

Note:
i.v.: intravenous injection,
i.v./once: administration once;

4. Assay method

In this assay, human breast cancer MDA-MB-468 cells (human breast cancer cells) were used to construct a xenograft tumor model in mice. When the tumor volume reached approximately 150 mm³, the animals were divided into 5 groups (d0) based on tumor volume according to Table 18, with 8 animals in the control group and 5 animals in the experimental group. Intravenous administration was given once, with an administration volume of 10 mL/kg. The vehicle control group (Vehicle) received a 0.9% sodium chloride injection. The assay was terminated on day 28 after the administration. The anti-tumor effects of four ADC drugs in vivo were investigated.
5. Observation indicator
5.1 Evaluation indicator

See the section of Assay 2: 5.1 Evaluation indicator
6 Result
6.1 Tumor volume

At the end of the assay, compared to the vehicle group, the tumor inhibition rates of SWY2110-JSSW-001 (DAR=7.12), SWY2111-JSSW-001 ADC (DAR=7.48), SWY2112-JSSW-001 ADC (DAR=7.51) and SWY2113-JSSW-001 ADC (DAR=7.50) experimental groups reached 65.8%, 65.1%, 58.3%, 47.1% (P<0.001) respectively, all demonstrating good tumor inhibition activity. It was suggested that four ADC drugs had significant inhibitory effect on human breast cancer MDA-MB-468 tumor cells. For details, see FIG. 20 and Table 19.

TABLE 19
Tumor parameters of each group 28 days after
administration of SWY2110 to SWY2113-ADCs

| | | | | Tumor | TGI |
|---|---|---|---|---|---|
| | Animal | Dosage | Administration | Volume | (%) |
| Group | Number | (mg/kg) | Route/Frequency | (mm³) | Day 28 |
| Vehicle | 8 | — | — | 525.0 ± 28.4 | — |
| SWY2110- | 5 | 2 | i.v./once | 275.9 ± 50.6*** | 65.8 |
| JSSW-001 ADC | | | | | |
| (DAR = 7.12) | | | | | |
| SWY2111- | 5 | 2 | i.v./once | 278.8 ± 22.9*** | 65.1 |
| JSSW-001 ADC | | | | | |
| (DAR = 7.48) | | | | | |
| SWY2112- | 5 | 2 | i.v./once | 304.3 ± 40.6*** | 58.3 |
| JSSW-001 ADC | | | | | |
| (DAR = 7.51) | | | | | |
| SWY2113- | 5 | 2 | i.v./once | 346.9 ± 46.2*** | 47.1 |
| JSSW-001 ADC | | | | | |
| (DAR = 7.50) | | | | | |

***compared with the vehicle group P < 0.001

### Assay 12 Efficacy Assay of SWY2110-ADC, SWY2111-ADC, SWY2112-ADC, SWY2113-ADC on Human Colorectal Cancer DiFi (Human Colorectal Cancer Cell) Xenograft Tumor

1. Assay animals

NU/NU mice, 5-6 weeks old, 35 mice
2. Assay object

To investigate the anti-tumor effect of SWY2110-JSSW-001, SWY2111-JSSW-001, SWY2112-JSSW-001, SWY2113-JSSW-001 drugs in vivo using a human colorectal cancer DiFi (human colorectal cancer cell) xenograft tumor model in NU/NU mice.
3. Drug dosage and grouping

TABLE 20
Table of animal grouping and dosage

| | Animal | Dosage | Administration |
|---|---|---|---|
| Group | Number | (mg/kg) | Route/Frequency |
| Vehicle | 7 | — | — |
| SWY2110-JSSW-001 | 7 | 1 | i.v./qw*twice |
| ADC (DAR = 7.12) | | | |
| SWY2111-JSSW-001 | 7 | 1 | i.v./qw*twice |
| ADC (DAR = 7.48) | | | |
| SWY2112-JSSW-001 | 7 | 1 | i.v./qw*twice |
| ADC (DAR = 7.51) | | | |
| SWY2113-JSSW-001 | 7 | 1 | i.v./qw*twice |
| ADC (DAR = 7.50) | | | |

Note:
i.v.: intravenous injection,
i.v./qw*twice: administration twice weekly;

4. Assay method

In this assay, human colorectal cancer DiFi cells (human colorectal cancer cells) were used to construct a xenograft tumor model in mice. When the tumor volume reached approximately 100 mm³, the animals were divided into 5 groups (d0) based on tumor volume according to Table 20, with 7 animals in each group. Intravenous administration was given twice, with an administration volume of 10 mL/kg. The vehicle control group (Vehicle) received a 0.9% sodium chloride injection. The assay was terminated on day 21 after the administration. The anti-tumor effects of four ADC drugs in vivo were investigated.
5. Observation indicator
5.1 Evaluation indicator

See the section of Assay 2: 5.1 Evaluation indicator
6 Result
6.1 Tumor volume

At the end of the assay, compared to the vehicle group, the tumor inhibition rates of SWY2110-JSSW-001 (DAR=7.12), SWY2111-JSSW-001 ADC (DAR=7.48), SWY2112-JSSW-001 ADC (DAR=7.51), SWY2113-JSSW-001 ADC (DAR=7.50) experimental groups reached 86.5%, 82.3%, 83.5%, 76.8% (P<0.001) respectively, all demonstrating good tumor inhibition activity. It was suggested that four ADC drugs had significant inhibitory effect on human colorectal cancer DiFi tumor cells. For details, see FIG. 21 and Table 21.

TABLE 21
Tumor parameters of each group 21 days after
administration of SWY2110 to SWY2113-ADCs

| | | | | Tumor | TGI |
|---|---|---|---|---|---|
| | Animal | Dosage | Administration | Volume | (%) |
| Group | Number | (mg/kg) | Route/Frequency | (mm³) | Day 28 |
| Vehicle | 7 | 0 | — | 562.2 ± 272.8 | — |
| SWY2110- | 7 | 1 | i.v./qw*twice | 161.9 ± 41.2*** | 86.5 |
| JSSW-001 ADC | | | | | |
| (DAR = 7.12) | | | | | |
| SWY2111- | 7 | 1 | i.v./qw*twice | 180.3 ± 98.8*** | 82.3 |
| JSSW-001 ADC | | | | | |
| (DAR = 7.48) | | | | | |
| SWY2112- | 7 | 1 | i.v./qw*twice | 175.3 ± 36.7*** | 83.5 |
| JSSW-001 ADC | | | | | |
| (DAR = 7.51) | | | | | |
| SWY2113- | 7 | 1 | i.v./qw*twice | 205.3 ± 155.6*** | 76.8 |
| JSSW-001 ADC | | | | | |
| (DAR = 7.50) | | | | | |

***compared with the vehicle group P < 0.001

### Assay 13: Efficacy Assay of SWY2110-ADC, SWY2111-ADC, SWY2112-ADC, SWY2113-ADC on Human Lung Cancer PC9-GR (Human Lung Cancer Gefitinib-Resi Stance Acquired Cell Line) Xenograft Tumor

1. Assay animals

NU/NU mice, 5-6 weeks old, 30 mice
2. Assay object

To investigate the anti-tumor effect of SWY2110-JSSW-001, SWY2111-JSSW-001, SWY2112-JSSW-001, SWY2113-JSSW-001 drugs in vivo using a human lung cancer cell PC9-GR xenograft tumor model in NU/NU mice.
3. Drug dosage and grouping

TABLE 22
Table of animal grouping and dosage

| | Animal | Dosage | Administration |
|---|---|---|---|
| Group | Number | (mg/kg) | Route/Frequency |
| Vehicle | 6 | — | — |
| SWY2110-JSSW-001 ADC | 6 | 3 | i.v./once |
| (DAR = 7.12) | | | |
| SWY2111-JSSW-001 ADC | 6 | 3 | i.v./once |
| (DAR = 7.48) | | | |
| SWY2112-JSSW-001 ADC | 6 | 3 | i.v./once |
| (DAR = 7.51) | | | |
| SWY2113-JSSW-001 ADC | 6 | 3 | i.v./once |
| (DAR = 7.50) | | | |

Note:
i.v.: intravenous injection,
i.v./once: administration once;

4. Assay method

In this assay, human lung cancer cell PC9-GR cells were used to construct a xenograft tumor model in mice. When the tumor volume reached approximately 110 mm³, the animals were divided into 5 groups (d0) based on tumor volume according to Table 22, with 6 animals in each group. Intravenous administration was given once, with an administration volume of 10 mL/kg. The vehicle control group (Vehicle) received a 0.9% sodium chloride injection. The assay was terminated on day 28 after the administration. The anti-tumor effects of four ADC drugs in vivo were investigated.
5. Observation indicator
5.1 Evaluation indicator

See the section of Assay 2: 5.1 Evaluation indicator
6 Result
6.1 Tumor volume

At the end of the assay, compared to the vehicle group, the tumor inhibition rates of SWY2110-JSSW-001 (DAR=7.12), SWY2111-JSSW-001 ADC (DAR=7.48), SWY2112-JSSW-001 ADC (DAR=7.51), SWY2113-JSSW-001 ADC (DAR=7.50) experimental groups reached 99.0%, 100.1%, 92.4%, 89.3% (P<0.001) respectively, all demonstrating good tumor inhibitory effect. It was suggested that four ADC drugs had significant inhibitory effect on human lung cancer cell PC9-GR tumor cells. For details, see FIG. 22 and Table 23.

TABLE 23
Tumor parameters of each group 28 days after
administration of SWY2110 to SWY2113-ADCs

| | | | | Tumor | TGI |
|---|---|---|---|---|---|
| | Animal | Dosage | Administration | Volume | (%) |
| Group | Number | (mg/kg) | Route/Frequency | (mm³) | Day 28 |
| Vehicle | 6 | — | — | 1159.3 | — |
| SWY2110-JSSW- | 6 | 3 | i.v./once | 124.3*** | 99.0 |
| 001 ADC | | | | | |
| (DAR = 7.12) | | | | | |
| SWY2111-JSSW- | 6 | 3 | i.v./once | 112.9*** | 100.1 |
| 001 ADC | | | | | |
| (DAR = 7.48) | | | | | |
| SWY2112-JSSW- | 6 | 3 | i.v./once | 193.0*** | 92.4 |
| 001 ADC | | | | | |
| (DAR = 7.51) | | | | | |
| SWY2113-JSSW- | 6 | 3 | i.v./once | 225.4*** | 89.3 |
| 001 ADC | | | | | |
| (DAR = 7.50) | | | | | |

***compared with the vehicle group P < 0.001

Assay 14 Efficacy Assay of antibody-drug conjugate on human lung cancer PC9-DTC (Del19/T790M/C797S, osimertinib-resistant cell) xenograft tumor in NOD-SCID mice
1. Assay animals

NOD-SCID mice, 5-6 weeks old, 18 mice
2. Assay object

To investigate the anti-tumor effect of two patritumab-ADC drugs in vivo using a human lung cancer PC9-DTC cell (Dell 9/T790M/C797S, osimertinib-resistant cell) xenograft tumor model in NU/NU mice.
3. Drug dosage and grouping

TABLE 24
Table of animal grouping and dosage

| | Animal | Dosage | Administration |
|---|---|---|---|
| Group | Number | (mg/kg) | Route/Frequency |
| Vehicle | 6 | 0 | i.v./qw*3w |
| patritumab-DXD ADC | 6 | 10 | i.v./qw*3w |
| (DAR = 7.3) | | | |
| patritumab-JSSW-001 ADC | 6 | 10 | i.v./qw*3w |
| (DAR = 7.36) | | | |

Note:
i.v.: intravenous injection,
qw*3w: administration once weekly for three weeks.

4. Assay method

In this assay, human lung cancer PC9-DTC cells (Del19/T790M/C797S, osimertinib-resistant cells) were used to construct a xenograft tumor model in nude mice. When the tumor volume reached approximately 115 mm³, the animals were divided into 3 groups (d0) based on tumor volume according to Table 24, with each 6 animals in the vehicle group, the patritumab-DXD ADC (DAR=7.3) group and patritumab-JSSW-001 ADC (DAR=7.36) group. Intravenous administration was given once weekly for 3 weeks, with an administration volume of 10 mL/kg. The vehicle control group (Vehicle) received a 0.9% sodium chloride injection. The assay was terminated on day 26 after the administration. The inhibitory effects of patritumab-DXD ADC (DAR=7.3) and patritumab-JSSW-001 ADC (DAR=7.36) on human lung cancer PC9-DTC (Del19/T790M/C797S, osimertinib-resistant cell) xenograft tumor in nude mice were compared.
5. Observation indicator
5.1 Evaluation indicator

See the section of Assay 2: 5.1 Evaluation indicator
6 Result
6.1 Tumor volume

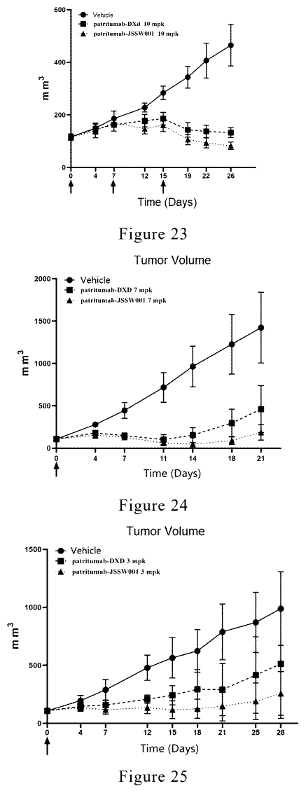

At the end of the assay, the patritumab-JSSW-001 ADC (DAR=7.36) group (P<0.001) exhibited the superior effect of inhibiting the volume of xenograft tumor compared to the patritumab-DXD ADC (DAR=7.3) (P<0.001) group. For details, see FIG. 23 and Table 25

TABLE 25
Tumor parameters of each group 26 days after administration of patritumab-ADC

| | | | | Tumor | TGI |
|---|---|---|---|---|---|
| | Animal | Dosage | Administration | Volume | (%) |
| Group | Number | (mg/kg) | Route/Frequency | (mm³) | Day 26 |
| Vehicle | 6 | 0 | i.v./qw*3w | 465.1 ± 78.9 | — |
| patritumab- | 6 | 10 | i.v./qw*3w | 132.9 ± 18.8*** | 71.3 |
| DXD ADC | | | | | |
| (DAR = 7.3) | | | | | |
| patritumab- | 6 | 10 | i.v./qw*3w | 82.9 ± 14.4*** | 82.3 |
| JSSW-001 ADC | | | | | |
| (DAR = 7.36) | | | | | |

***compared with the vehicle group P < 0.001

### Assay 15 Efficacy Assay of Antibody-Drug Conjugates on Human Colon Cancer SW620 Xenograft Tumor in NU/NU Mice

1. Assay animals

NU/NU mice, 5-6 weeks old, 18 mice
2. Assay object

To investigate the anti-tumor effect of two patritumab-ADC drugs in vivo using a human colon cancer SW620 cell xenograft tumor model in NU/NU mice.
3. Drug dosage and grouping

TABLE 26
Table of animal grouping and dosage

| | Animal | Dosage | Administration |
|---|---|---|---|
| Group | Number | (mg/kg) | Route/Frequency |
| Vehicle | 6 | 0 | i.v./single |
| | | | administration |
| patritumab-DXD ADC | 6 | 7 | i.v./single |
| (DAR = 7.3) | | | administration |
| patritumab-JSSW-001 ADC | 6 | 7 | i.v./single |
| (DAR = 7.36) | | | administration |

4. Assay method

In this assay, human colon cancer SW620 cells were used to construct a xenograft tumor model in nude mice. When the tumor volume reached approximately 110 mm³, the animals were divided into 3 groups (d0) based on tumor volume according to Table 26, with each 6 animals in the vehicle group, the patritumab-DXD ADC (DAR=7.3) group and the patritumab-JSSW-001 ADC (DAR=7.36) group. Intravenous administration was given once, with an administration volume of 10 mL/kg. The vehicle control group (Vehicle) received a 0.9% sodium chloride injection. The assay was terminated on day 26 after the administration. The inhibitory effects of patritumab-DXD ADC (DAR=7.3) and patritumab-JSSW-001 ADC (DAR=7.36) on human colon cancer SW620 xenograft tumor in nude mice were compared.
5. Observation indicator
5.1 Evaluation indicator

See the section of Assay 2: 5.1 Evaluation indicator
6 Result
6.1 Tumor volume

At the end of the assay, the patritumab-JSSW-001 ADC (DAR=7.36) group (P<0.001) exhibited the superior effect of inhibiting the volume of xenograft tumor compared to the patritumab-DXD ADC (DAR=7.3) (P<0.001) group. For details, see FIG. 24 and Table 27.

TABLE 27
Tumor parameters of each group 21 days after administration of patritumab-ADC

| | | | | Tumor | TGI |
|---|---|---|---|---|---|
| | Animal | Dosage | Administration | Volume | (%) |
| Group | Number | (mg/kg) | Route/Frequency | (mm³) | Day 21 |
| Vehicle | 6 | 0 | i.v./single | 1422.8 ± 416.0 | — |
| | | | administration | | |
| patritumab- | 6 | 7 | i.v./single | 461.3 ± 278.1*** | 73.2 |
| DXD ADC | | | administration | | |
| (DAR = 7.3) | | | | | |
| patritumab- | 6 | 7 | i.v./single | 187.5 ± 91.6*** | 94.0 |
| JSSW-001 ADC | | | administration | | |
| (DAR = 7.36) | | | | | |

***compared to the vehicle control group P < 0.001

### Assay 16 Efficacy Assay of Antibody-Drug Conjugates on Human Colon Cancer DiFi Xenograft Tumor in NU/NU Mice

1. Assay animals

NU/NU mice, 5-6 weeks old, 18 mice
2. Assay object

To investigate the anti-tumor effect of two patritumab-ADC drugs in vivo using a human colorectal cancer DiFi cell xenograft tumor model in NU/NU mice.
3. Drug dosage and grouping

TABLE 28
Table of animal grouping and dosage

| | Animal | Dosage | Administration |
|---|---|---|---|
| Group | Number | (mg/kg) | Route/Frequency |
| Vehicle | 6 | 0 | i.v./single |
| | | | administration |
| patritumab-DXD ADC | 6 | 3 | i.v./single |
| (DAR = 7.3) | | | administration |
| patritumab-JSSW-001 ADC | 6 | 3 | i.v./single |
| (DAR = 7.36) | | | administration |

Note:
i.v.: intravenous injection.

4. Assay method

In this assay, human colorectal cancer DiFi cells were used to construct a xenograft tumor model in nude mice. When the tumor volume reached approximately 1 10 mm³, the animals were divided into 3 groups (d0) based on tumor volume according to Table 28, with 6 animals in vehicle group, each 5 animals in patritumab-DXD ADC (DAR=7.3) group and patritumab-JSSW-001 ADC (DAR=7.36) group. Intravenous administration was given once, with an administration volume of 10 mL/kg. The vehicle control group (Vehicle) received a 0.9% sodium chloride injection. The assay was terminated on day 28 after the administration. The inhibitory effects of patritumab-DXD ADC (DAR=7.3) and patritumab-JSSW-001 ADC (DAR=7.36) on human colorectal cancer DiFi xenograft tumor in nude mice were compared.
5. Observation indicator
5.1 Evaluation indicator

See the section of Assay 2: 5.1 Evaluation indicator
6 Result
6.1 Tumor volume

At the end of the assay, the patritumab-JSSW-001 ADC (DAR=7.36) group (P<0.01) exhibited the superior effect of inhibiting the volume of xenograft tumor compared to the patritumab-DXD ADC (DAR=7.3) (P<0.05) group. For details, see FIG. 25 and Table 29.

TABLE 29
Tumor parameters of each group 28 days after administration of patritumab-ADC

| | | | | Tumor | TGI |
|---|---|---|---|---|---|
| | Animal | Dosage | Administration | Volume | (%) |
| Group | Number | (mg/kg) | Route/Frequency | (mm³) | Day 28 |
| Vehicle | 6 | 0 | i.v./qw*3w | 989.8 ± 317.6 | — |
| patritumab-DXD | 6 | 10 | i.v./qw*3w | 512.5 ± 470.8* | 54.0 |
| ADC (DAR = 7.3) | | | | | |
| patritumab- | 6 | 10 | i.v./qw*3w | 257.9 ± 189.1** | 82.9 |
| JSSW-001 ADC 1 | | | | | |
| (DAR = 7.36) | | | | | |

*compared with the vehicle group P < 0.05,
**compared with the vehicle group P < 0.01

### Assay 17: Safety Evaluation Study

In this assay, six appropriately aged crab-eating macaques were selected, with three males and three females, and were intravenously injected with SWY2110-JSSW-001, SWY2111-JSSW-001, and SWY2113-JSSW-001. The administration regimen was designed as shown in the table below, with intravenous injections once every three weeks for three successive injections. After the administration, a continuous observation was made for 7 days, a general observation was made twice daily, and a detailed observation was made once daily. Changes in body weight were observed before and after administration, and hematological tests were performed. Assay results showed that compared to the ADCs prepared with pH-dependent antibodies (SWY2111-JSSW-001 and SWY2113-JSSW-001), SWY2110-JSSW-001 exhibited reduced toxicity in skin and gastrointestinal tracts, and no obvious target-related toxicity (such as skin ulceration, scabbing, loose stools, etc.), indicating that the pH-modified ADCs had reduced toxicity to normal tissues, however, there was no significant difference in the haematological toxicity caused by the three ADC molecules.

TABLE 30
Pre-toxicology experiment design scheme

| | Administration frequency | | | |
|---|---|---|---|---|
| | Q3W × 3 | | | |
| | Group | D1 | D22 | D43 |
| Administration | Group 1: SWY2110- | 6 mg/kg | 30 mg/kg | 30 mg/kg |
| dosage | JSSW-001 | | | |
| | Group 2: SWY2111- | 6 mg/kg | 30 mg/kg | 30 mg/kg |
| | JSSW-001 | | | |
| | Group 3: SWY2113- | 6 mg/kg | 30 mg/kg | 30 mg/kg |
| | JSSW-001 | | | |

Animal number 1 animal/sex/group

TABLE 31
Pre-toxicity assay results

| | |
|---|---|
| General | Group 1 showed loose stools, skin flakes on |
| observation | limbs, ulceration, and scabbing; |
| | Group 2 showed soft stools, mild skin flakes, |
| | and no ulceration; |
| | Group 3 showed no abnormalities. |
| Body Weight | Group 1 showed a decrease in body weight within |
| | 7 days after each administration, with partial |
| | recovery before the next administration; |
| | Group 2 showed a slight decrease in body weight |
| | within 7 days after each administration, with |
| | full recovery before the next administration; |
| | Group 3 showed no significant decrease in body |
| | weight within 7 days after each administration. |
| Hematology | Group 1 HGB, WBC, LYMP↓; |
| | Group 2 HGB, WBC, LYMP↓; |
| | Group 3 HGB, WBC, LYMP↓; |

HGB: haemoglobin; WBC: white blood count; LYMP: lymphocyte absolute number.

### FORMULATION EXAMPLES

### Formulation Example 1

The pH value is an important factor affecting the stability of biological products. It regulates the charge distribution on the protein surface, thereby affecting intermolecular and intramolecular forces of the protein, and influencing the protein conformation and colloidal stability, while also affecting the chemical stability of the protein, such as degradation reactions like deamidation. An appropriate amount of buffer salt has the function of maintaining a stable pH of the solution; the type of buffer salt plays an important role in the physicochemical properties and stability of biological products.

Histidine-histidine hydrochloride and citric acid-sodium citrate were selected as candidate buffer salts, with a buffer salt ion concentration of 25 mM. The pH value of the histidine-histidine hydrochloride buffer salt was adjusted to 5.0, 5.5, 6.0, and 6.5, and the pH of the citric acid-sodium citrate buffer salt was adjusted to 5.5, 6.0, 6.5, and 7.0. Sucrose was added to each buffer solution to prepare 8 groups of solutions (at 5% sucroce concentration) with different pH values and buffer salts. The antibody-drug conjugate (SWY2110-JSSW-001) sample was exchanged into the above 8 groups of solutions through ultrafiltration, with a final concentration of the antibody-drug conjugate of approximately 5 mg/ml. The appearance of the samples after ultrafiltration exchanging was observed. The histidine-histidine hydrochloride (pH 6.0, 6.5) groups and the citric acid-sodium citrate (pH 5.5, 6.0) groups all showed significant turbidity, and the antibody-drug conjugates precipitated; these 4 groups of samples were discarded. The remaining 4 groups of samples were placed under high temperature (40±2° C.) conditions and sampled on the 7th and 14th days; they were placed under light exposure (5±3° C., white light illuminance: 5000 Lux, UV intensity: 90 μw/cm²) conditions and sampled on the 10th day. The protein concentrations of the antibody-drug conjugates were detected by ultraviolet spectrophotometry, and the molecular size variants of the antibody-drug conjugates were detected by size exclusion chromatography (SEC-HPLC) to investigate the stability of the antibody-drug conjugate in the 4 groups of buffer solutions after high temperature and light exposure treatment. After 7 days at high temperature, the pH 5.5 test group showed protein precipitation with a significant decrease in protein concentration, and a significant decrease in monomer using SEC. After 10 days of light exposure, the citric acid test group showed white precipitate with a significant decrease in protein concentration, and a significant decrease in monomer using SEC. In summary, the 25 mM histidine-histidine hydrochloride at pH=5.0 was selected as the target buffer.

### Detection Methods:

Detection of protein concentration: the protein concentration was detected using a Nanodrop2000 microspectrophotometer, using a protein-free placebo for zero calibration, based on the extinction coefficient of the antibody-drug conjugate of 1.25 1.

SFC-HPLC: According to Chinese Pharmacopoeia 2020 edition, Part IV, General Rule 0512 High Performance Liquid Chromatography, analysis and detection were performed using a chromatographic column packed with a gel suitable for separating proteins having a molecular weight of 10-500 kD. The proportions of the protein monomer peak, the high molecular weight substance (HMWS) peak and the low molecular weight substance (LMWS) peak were calculated according to the area normalization method.

Detection result is shown below:

TABLE 31
Appearance result

| | | Appearance | | | | |
|---|---|---|---|---|---|---|
| | | | 5 °C, | | | |
| Buffer salt | | | protected | 5 °C, light | 40 °C, high | 40 °C, high |
| (containing | pH | | from light, | exposure, | temperature, | temperature, |
| 5% sucrose) | value | 0 d | 10 d | 10 d | 7 d | 14 d |
| Histidine- | 5.0 | Clear | Clear | Clear | Clear | Clear |
| histidine | 5.5 | Clear | Clear | Clear | Slight | More |
| hydrochloride | | | | | protein | protein |
| | | | | | precipitate | precipitate |
| Citric acid- | 6.5 | Clear | Clear | Slight | Clear | Clear |
| sodium | | | | protein | | |
| citrate | | | | precipitate | | |
| | 7.0 | Clear | Clear | Slight | Clear | Clear |
| | | | | protein | | |
| | | | | precipitate | | |

TABLE 32
Detection result of protein concentrations

| | | Protein concentrations (mg/ml) | | | | |
|---|---|---|---|---|---|---|
| | | | 5 °C, | | | |
| | | | protected | 5 °C, | | |
| Buffer salt | | | from | light | 40 °C, high | 40 °C, high |
| (containing 5% | pH | | light, | exposure, | temperature, | temperature, |
| sucrose) | value | 0 d | 10 d | 10 d | 7 d | 14 d |
| Histidine-histidine | 5.0 | 5.56 | 5.30 | 5.35 | 5.32 | 5.47 |
| hydrochloride | 5.5 | 4.72 | 4.57 | 4.61 | 3.24 | 2.49 |
| Citric acid-sodium | 6.5 | 5.58 | 5.39 | 3.49 | 5.48 | 5.44 |
| citrate | 7.0 | 5.44 | 5.45 | 3.85 | 5.49 | 5.52 |

TABLE 33
Detection result of SEC-HPLC

| | | SEC-HPLC concentrations (%) | | | | | | | | |
|---|---|---|---|---|---|---|---|---|---|---|
| | | | | | 40 °C, high | | | 5 °C, light | | |
| Buffer | | 0 d | | | temperature, 7 d | | | exposure, 10 d | | |
| (containing | pH | | Main | | | Main | | | Main | |
| 5% sucrose) | value | Aggrelgate | peak | Fraglment | Aggrelgate | peak | Fraglment | Aggrelgate | peak | Fraglment |
| Histidine- | 5.0 | 1.57 | 97.30 | 1.13 | 3.77 | 94.41 | 1.82 | 5.17 | 93.61 | 1.23 |
| histidine | 5.5 | 1.33 | 97.55 | 1.12 | 12.72 | 85.10 | 2.18 | 8.63 | 89.87 | 1.51 |
| hydrochloride | | | | | | | | | | |
| Citric acid- | 6.5 | 3.53 | 95.30 | 1.17 | 8.39 | 89.98 | 1.65 | 23.06 | 75.08 | 1.87 |
| sodium citrate | 7.0 | 4.5 | 94.41 | 1.10 | 9.13 | 89.06 | 1.81 | 47.66 | 50.89 | 1.47 |

After replacing SWY2110-JSSW-001 with the antibody-drug conjugates SWY2111-JSSW-001 and SWY2113-JSSW-001, the stability of the antibody-drug conjugates in the buffer was investigated using the same method described above. The test results were substantially similar to those of SWY2110-JSSW-001. The 25 mM histidine-histidine hydrochloride at pH=5.0 was selected as the target buffer.

### Formulation Example 2

Saccharides are widely used stabilizers in monoclonal antibody and ADC products, serving to stabilize monoclonal antibody drugs, and can also be used as excipients for lyophilized products, while also having the function of adjusting the osmotic pressure of the product.

The 25 mM histidine-histidine hydrochloride buffer at pH=5.0 was prepared, and sucrose or trehalose was added separately to prepare 4 groups of solutions with different saccharides and saccharide concentrations (5% or 9%). The antibody-drug conjugate (SWY2110-JSSW-001) sample was exchanged into the above 4 groups of solutions through ultrafiltration, with a final concentration of the antibody-drug conjugate of approximately 10 mg/ml. The thermal stability of the initial samples was detected by differential scanning calorimetry (DSC). The samples were placed under high temperature (40±2° C.) conditions and sampled on the 7th and 14th days; they were placed under light exposure (5±3° C., white light illuminance: 5000 Lux, UV intensity: 90 μw/cm²) conditions and sampled on the 10th day. The molecular size variants of the antibody-drug conjugates were detected by size exclusion chromatography (SEC-HPLC), and the charge variants of the antibody-drug conjugates were detected by ion exchange chromatography (CEX-HPLC) to investigate the stability of the antibody-drug conjugates in the 8 groups of solutions after high temperature and light exposure treatments. Additionally, under the same lyophilization process, the appearances of two lyophilized powders with 9% sucrose and trehalose were compared.

DSC test results showed that Tₒₙₛₑₜ and Tm₂ of the test groups with 9% saccharide added were significantly higher than those of the test groups with 5% saccharide. There was no significant difference between sucrose and trehalose. The SEC test results indicated that when the saccharide concentration was raised from 5% to 9%, the level of SEC monomer content remained significantly high. The CEX test results showed that in the high temperature test, neither the addition of saccharide nor its concentration had a significant effect on the main peak purity; whereas in the light exposure test, significant changes occurred in the acidic and basic variants of the sample, and the chromatogram thereof could no longer distinguish the acidic peak, main peak, and basic peak. Therefore, the CEX data were not statistically analyzed. Comparing the morphology of lyophilized powders with different stabilizers, the shrinkage at the bottom of lyophilized powder from the sucrose test group was significantly more severe than that from the trehalose test group. In summary, the addition of 9% trehalose was selected for the stabilizer.

### Detection Method

DSC: A sample to be tested was diluted with the same buffer therein to a protein concentration of 1 mg/mL. Then, 350 μL each of the test sample and the corresponding buffer were added to the designated wells of a 96-well plate. The 96-well plate was placed into a sample holder, and the temperature of the sample holder was set to 15° C. Instrument parameters were set as follows: initial temperature: 20° C., final temperature: 100° C., and heating rate: 90° C./min.

SEC-HPLC: Detection method was identical to that in Formulation Example 1.

CEX-HPLC: According to Chinese Pharmacopoeia 2020 edition, Part IV, General Rule 0512 High Performance Liquid Chromatography, analysis and detection were performed using a cation exchange column as the chromatographic column. The proportions of the acidic peak, main peak, and basic peak of the test sample were calculated according to the area normalization method.

Detection results:

TABLE 34
DSC detection results

| Item | Tₒₙₛₑₜ(°C) | Tm₁(°C) | Tm₂(°C) | Tm₃(°C) |
|---|---|---|---|---|
| pH 5.0 5% sucrose | 48.87 | 58.45 | 70.24 | 79.17 |
| pH 5.0 9% sucrose | 49.61 | 59.55 | 70.82 | 79.70 |
| pH 5.0 5% trehalose | 48.77 | 58.19 | 70.24 | 79.18 |
| pH 5.0 9% trehalose | 49.22 | 59.09 | 70.82 | 79.76 |

TABLE 35
SEC-HPLC detection result

| | SEC-HPLC purity (%) | | | | | | | | | | | |
|---|---|---|---|---|---|---|---|---|---|---|---|---|
| | | | | 40 °C, high | | | 40 °C, high | | | 5 °C, light | | |
| | 0 d | | | temperature, 7 d | | | temperature, 14 d | | | exposure, 10 d | | |
| | | Main | | | Main | | | Main | | | Main | |
| Item | Aggregate | peak | Fragment | Aggregate | peak | Fragment | Aggregate | peak | Fragment | Aggregate | peak | Fragment |
| pH 5.0 | 2.20 | 95.83 | 1.97 | 6.21 | 91.11 | 2.67 | 11.45 | 84.75 | 3.81 | 10.75 | 86.93 | 2.32 |
| 5% | | | | | | | | | | | | |
| sucrose | | | | | | | | | | | | |
| pH 5.0 | 2.16 | 95.88 | 1.96 | 5.01 | 92.42 | 2.58 | 7.99 | 88.35 | 3.66 | 9.69 | 87.34 | 2.97 |
| 9% | | | | | | | | | | | | |
| sucrose | | | | | | | | | | | | |
| pH 5.0 | 2.28 | 95.69 | 2.02 | 7.92 | 89.51 | 2.57 | 11.29 | 84.93 | 3.77 | 10.89 | 86.18 | 2.93 |
| 5% | | | | | | | | | | | | |
| trehalose | | | | | | | | | | | | |
| pH 5.0 | 2.06 | 95.88 | 2.05 | 5.55 | 91.82 | 2.63 | 8.18 | 88.07 | 3.76 | 10.65 | 86.12 | 3.23 |
| 9% | | | | | | | | | | | | |
| trehalose | | | | | | | | | | | | |

TABLE 36
CEX-HPLC detection result

| | CEX-HPLC concentration (%) | | | | | | | | |
|---|---|---|---|---|---|---|---|---|---|
| | | | | 40 °C, high | | | 40 °C, high | | |
| | 0 d | | | temperature, 7 d | | | temperature, 14 d | | |
| | Acidic | Main | Basic | Acidic | Main | Basic | Acidic | Main | Basic |
| Item | peak | peak | peak | peak | peak | peak | peak | peak | peak |
| pH 5.0 | 25.55 | 44.75 | 29.70 | 28.40 | 43.78 | 27.82 | 32.58 | 37.60 | 29.82 |
| 5% | | | | | | | | | |
| sucrose | | | | | | | | | |
| pH 5.0 | 26.44 | 43.99 | 29.57 | 28.33 | 43.89 | 27.78 | 32.84 | 37.38 | 29.78 |
| 9% | | | | | | | | | |
| sucrose | | | | | | | | | |
| pH 5.0 | 26.41 | 44.35 | 29.24 | 27.74 | 43.72 | 28.54 | 32.63 | 37.71 | 29.66 |
| 5% | | | | | | | | | |
| trehalose | | | | | | | | | |
| pH 5.0 | 26.43 | 44.80 | 28.77 | 27.74 | 44.65 | 27.61 | 33.17 | 37.53 | 29.30 |
| 9% | | | | | | | | | |
| trehalose | | | | | | | | | |

TABLE 37
Detection results for basic evaluation
indicators of lyophilized powders

| | | Moisture | |
|---|---|---|---|
| | Appearance of lyophilized | content | Reconstitution |
| Stabilizer | powder | (%) | time |
| 9% | White loose solid, slight | 1.45 | ≤1 min |
| trehalose | shrinkage at the bottom | | |
| 9% | White loose solid, moderately | 1.85 | ≤1 min |
| sucrose | severe shrinkage at the | | |
| | bottom | | |

After replacing SWY2110-JSSW-001 with the antibody-drug conjugates SWY2111-JSSW-001 and SWY2113-JSSW-001, the effect of the stabilizer on the antibody-drug conjugates was investigated using the same method described above. The test results were substantially similar to those of SWY2110-JSSW-001. Trehalose was selected as the stabilizer.

### Formulation Example 3

25 mM histidine-histidine hydrochloride buffer at pH=5.0 was prepared, and trehalose was added at 9%. The antibody-drug conjugate (SWY2110-JSSW-001) was exchanged into the above solution through ultrafiltration and polysorbate 80 was added to prepare samples with a final concentration of the antibody-drug conjugate of approximately 10 mg/ml. The samples were dispensed into injection vials, and a stability study was conducted under freezing-thawing conditions (frozen at −20° C. and thawed at room temperature for a total of 6 cycles). Changes in subvisible particles in samples under different conditions were detected using a microflow imaging instrument (Flowcam). The results showed that adding polysorbate 80 as the surfactant could effectively control the increase in the number of particles. Adding 0.01% polysorbate 80 showed a significant inhibitory effect on the formation of particles. Adding 0.03% polysorbate 80 as the surfactant was preferred.

TABLE 38
Effect of freezing-thawing on insoluble particles in samples
with different addition amounts of polysorbate 80

| | | PS80 Content | | | | | | | |
|---|---|---|---|---|---|---|---|---|---|
| | | 0 | | 0.01% | | 0.03% | | 0.05% | |
| | | | 6 cycles | | 6 cycles | | 6 cycles | | 6 cycles |
| | | | of | | of | | of | | of |
| | | | freezing- | | freezing- | | freezing- | | freezing- |
| Particle | | T0 | thawing | T0 | thawing | T0 | thawing | T0 | thawing |
| Particle | NSP 2- | 2481 | 15799 | 62 | 72 | 49 | 119 | 7 | 25 |
| number | 10 μm | | | | | | | | |
| (P/mL) | NSP | 1008 | 15410 | 7 | 15 | 0 | 10 | 0 | 5 |
| | 10 + μm | | | | | | | | |

After replacing SWY2110-JSSW-001 with the antibody-drug conjugates SWY2111-JSSW-001 and SWY2113-JSSW-001, the effect of the surfactant on the stability of the antibody-drug conjugates was investigated using the same method described above. The test results were substantially similar to those of SWY2110-JSSW-001. Polysorbate 80 was selected as the surfactant.

### Formulation Example 4

A formula of 10 mg/ml of the antibody-drug conjugate (SWY2110-JSSW-001), 25 mmol/L of histidine-histidine hydrochloride, 0.03% of polysorbate 80, and 9% of trehalose at pH=5.0 was selected as base formula. The glass transition temperature and collapse temperature of this base formula were detected. Low-temperature DSC detection showed the glass transition temperature of the sample was −29.28° C., and the lyophilization microscopy detection showed the collapse temperature of the sample was −25.7° C.

Pre-freezing: Based on the glass transition temperature, the pre-freezing temperature was set to −45° C. First, the shelf temperature was rapidly lowered to −5° C. and held at this temperature for 30 minutes. Then the product was cooled to the pre-freezing temperature of −45° C. either rapidly (2° C./min) or slowly (0.5° C./min) and maintained at this temperature for at least 180 minutes. The lyophilization processes were subsequently carried out under the same primary and secondary drying conditions. No significant difference in moisture contents of the lyophilized powders was observed between two pre-freezing methods. The rapid freezing led to the formation of smaller ice crystals, but it did not increase the moisture content compared with the slow freezing; therefore, the rapid freezing was preferably selected for the pre-freezing.

TABLE 39
Effect of rapid and slow freezing at different
positions on moisture content

| | Moisture content (%) | |
|---|---|---|
| | | slow freezing |
| Sample Position Number | rapid freezing (2 °C/min) | (0.5 °C/min) |
| A | 1.10 | 1.19 |
| B | 1.17 | 1.16 |
| C | 1.22 | 1.25 |
| D | 1.16 | 1.20 |
| E | 1.10 | 1.19 |
| Average | 1.44 | 1.39 |

Note:
C was the center position of the shelf, A was a corner of the shelf near the door, E was the diagonal corner of A near the isolation valve, B and D were the midpoints of AC and CE, respectively.

Primary drying: The shelf temperature was set above the allowable collapse temperature of the product, ensuring that the temperature of the non-sublimed portion of the product did not exceed its collapse temperature. The rapid freezing was preferably used in the pre-freezing process. At the primary drying temperature of −20° C., the lyophilization effects under the vacuum values of the lyophilization chamber of 0.20 mbar and 0.25 mbar were compared. Both batches of lyophilized powders were white cake-like loose solids, showing no collapse or significant shrinkage, with essentially identical moisture contents and reconstitution times. With essentially the same moisture contents of the lyophilized powders, the primary drying time at −20° C. and 0.20 mbar was 200 minutes shorter than that at −20° C. and 0.25 mbar. Therefore, the primary drying was preferably carried out at −20° C. and 0.20 mbar.

TABLE 40
Morphology, moisture content, and reconstitution time of lyophilized
powders under different primary drying pressures

| Batch number | DP221 | DP228 |
|---|---|---|
| Primary drying condition | −20 °C 0.25 mbar 4000 min | −20 °C 0.20 mbar 3800 min |
| Moisture content of sample A | 0.84% | 1.10% |
| Moisture content of sample B | 1.12% | 1.17% |
| Moisture content of sample C | 1.20% | 1.22% |
| Average moisture content | 1.09% | 1.16% |
| Lyophilized powder | White, cake-like, loose | White, cake-like, loose |
| morphology | solid | solid |
| Reconstitution time | ≤1 min | ≤1 min |

Note:
C was the center position of the shelf, A was a corner near the door, B was the midpoint of AC.

Secondary drying: After the completion of primary drying, the shelf temperature was set below the maximum allowable temperature for the product. The secondary drying temperature was set to 30° C. and the chamber pressure was set to 0.20 mbar, consistent with the primary drying condition.

The key qualities of the lyophilized samples were compared before and after lyophilization. The results showed no significant changes in product quality after lyophilization, indicating that the lyophilization process did not have a significant impact on the product quality.

TABLE 41
Impact of lyophilization process on the product quality

| | Stock solution batch number: | | Stock solution batch number: | |
|---|---|---|---|---|
| | DS218 | | DS228 | |
| | | Reconstitution | | Reconstitution |
| | Stock | solution of | Stock | solution of |
| | solution | lyophilized powder | solution | lyophilized powder |
| Item | (DS218) | (DP221) | (DS228) | (DP228) |
| Moisture | — | 1.1 | — | 1.2 |
| content (%) | | | | |
| Reconstitution | — | <3 min | — | <3 min |
| time (min) | | | | |
| pH value | 5.0 | 5.0 | 5.0 | 5.0 |
| Protein content | 10.1 | 9.8 | 9.9 | 10.2 |
| (mg/ml) | | | | |
| SEC-HPLC | HMWS: 1.2% | 1.1% | HMWS: 1.3% | 1.3% |
| | Monomer: 97.4% | 97.5% | Monomer: 96.7% | 96.6% |
| | LMWS: 1.4% | 1.4% | LMWS: 2.0% | 2.2% |
| CEX-HPLC | Acidic peak: 24.2% | 24.3% | Acidic peak: 22.5% | 22.5% |
| | Main peak: 47.5% | 48.4% | Main peak: 47.2% | 48.5% |
| | Basic peak: 28.3% | 27.3% | Basic peak: 30.3% | 29.0% |
| NR-CE-SDS | Monomer: 97.2% | 97.6% | Monomer: 97.1% | 96.5% |
| | LMWS: 2.8% | 2.4% | LMWS: 2.9% | 3.5% |
| DAR | DAR value: 8.0 | 8.0 | DAR value: 8.0 | 8.0 |
| Antigen | 86% | 106% | 100% | 105% |
| binding activity | | | | |
| Bioactivity | 94% | 96% | 100% | 114% |

After replacing SWY2110-JSSW-001 with the antibody-drug conjugates SWY2111-JSSW-001 and SWY2113-JSSW-001, the impact of the lyophilization process on the product quality was investigated using the same method described above. The test results were substantially similar to those of SWY2110-JSSW-001, indicating that the lyophilization process did not have a significant impact on the product quality.

### Formulation Example 5

Stability studies at 5±3° C. were carried out separately on the stock solution of the antibody-drug conjugate (SWY2110-JSSW-001) before lyophilization and the finished product of the antibody-drug conjugate (SWY2110-JSSW-001) after lyophilization. The study results showed that the quality of the product in the lyophilized state was more stable than that in the liquid state. The product in the liquid state showed significantly increased high molecular-weight fragments than the product in the lyophilized state. Moreover, the product in the liquid state was more prone to generating free small molecule payloads, with an obviously higher releasing rate compared to the lyophilized powder. The lyophilized dosage form was more conducive to the long-term storage of the product.

### Detection Methods:

SEC-HPLC: According to Chinese Pharmacopoeia 2020 edition, Part IV, General Rule 0512 High Performance Liquid Chromatography, analysis and detection were performed using a chromatographic column packed with a gel suitable for separating proteins with a molecular weight of 10-500 kD (e.g., XbridgeR BEH200Å, 7.8×300 mm, 200Å, 3.5 μm or other suitable chromatographic columns). The mobile phase included 100 mmol/L phosphate buffer, 100 mmol/L NaCl, and 10% isopropanol at pH=6.7±0.1, the flow rate was 0.8 mL/min, and the detection wavelength was 280 nm. An appropriate amount of the test sample solution was injected into the liquid chromatograph. The proportions of the protein monomer peak, the high molecular weight substance (HMWS) peak and the low molecular weight substance (LMWS) peak were calculated according to the area normalization method.

CEX-HPLC: According to Chinese Pharmacopoeia 2020 edition, Part IV, General Rule 0512 High Performance Liquid Chromatography, analysis and detection were performed using an ion exchange column (e.g., BioPro IEX SF, 4.6×100 mm or other suitable chromatographic columns). The mobile phase A included 20 mmol/L MES (morpholinoethanesulfonic acid) at pH=6.1, and the mobile phase B included 20 mmol/L MES and 500 mmol/L NaCl at pH=6.1, the flow rate was 1 mL/min, and the detection wavelength was 280 nm. An appropriate amount of the test sample solution was injected into the liquid chromatograph for gradient elution. The proportions of the acidic peak, main peak, and basic peak of the test sample were calculated according to the area normalization method.

NR-CE-SDS: Detection was performed according to Chinese Pharmacopoeia 2020 edition, Part IV, General Rule 3127 Determination of Molecular Size Variants of Monoclonal Antibodies. The proportions of the monomer peak and low molecular weight substance (LMWS) peak were calculated according to the area normalization method.

icIEF: Detection was performed according to Chinese Pharmacopoeia 2020 edition, Part IV, General Rule 3129, Determination of Charge Variants of Monoclonal Antibodies. Based on the isoelectric point (PI) characteristics of different charge variants, they were separated by capillary electrophoresis (General Rule 0542). The isoelectric points of the charge variants were determined and their relative percentages were calculated.

DAR distribution: According to Chinese Pharmacopoeia 2020 edition, Part IV, General Rule 0512 High Performance Liquid Chromatography, analysis and detection were performed using a hydrophobic chromatographic column (e.g., TSKgel Butyl-NPR, 4.6×100 mm or other suitable columns) as the chromatographic column. The mobile phase A included LOM ammonium sulfate and 25 mM phosphate buffer (pH=7.0), and the mobile phase B included 25% isopropanol and 75% 25 mM phosphate buffer (pH=7.0). The flow rate was 0.8 ml/min, and the detection wavelength was 280 nm. An appropriate amount of the test sample solution was injected into the liquid chromatograph for gradient elution. The DAR value was calculated.

Free small molecule payload content: According to Chinese Pharmacopoeia 2020 edition, Part IV, General Rule 0512 High Performance Liquid Chromatography and 0431 Mass Spectrometry, analysis and detection were performed using a chromatographic column packed with an octadecylsilyl silica gel (e.g., Waters ACQUITY UPLC® BEH-C18, 2.1×50 mm, particle size 1.7 μm or other suitable chromatographic columns). The mobile phase A was an aqueous solution containing 0.1% FA, and the mobile phase B was an acetonitrile solution containing 0.1% FA. The column temperature was 40° C., and the flow rate was 0.4 ml/min. 50 μL of the test sample (10 mg/mL) was taken, and an internal standard working solution was added. The mixture was precipitated with acetonitrile in the ratio of 1:3, placed in a −20° C. ice bath for 30 minutes, and then centrifuged. The supernatant was taken and loaded onto the column. Different concentrations of small molecule payloads were used as reference standards and loaded onto the column. After gradient elution by liquid chromatography, the sample was sent to the mass spectrometer detector. After setting parameters such as capillary and cone voltages, ion source temperature, collision energy, and nebulizer flow rate, the analysis was performed. The free small molecule payload content in the test sample was calculated according to the standard curve.

Free small molecule payloads content(%)=Detection concentration of free small molecule payloads/ADC concentration×100%

Antigen binding activity: Determined by enzyme-linked immunosorbent assay (ELISA).

Bioactivity: Determined by cell proliferation inhibition assay.

### Detection Results:

TABLE 42
Stability results for key quality indicators of GMP Batch 1

| | GMP Batch 1 | | | |
|---|---|---|---|---|
| | Stock solution (5 ± 3 °C) | | Lyophilized powder (5 ± 3 °C) | |
| Items | 0 months | 6 months | 0 months | 6 months |
| SEC-HPLC | HMWS: 1.4% | 2.1% | HMWS: 1.4% | 1.1% |
| | Monomer: 97.4% | 96.2% | Monomer: 97.4% | 97.4% |
| | LMWS: 1.3% | 1.7% | LMWS: 1.3% | 1.5% |
| NR-CE-SDS | LC + HC: 97.0% | 97.3% | HC + LC: 97.3% | 97.5% |
| | Other peak: 3.0% | 2.7% | Other peak: 2.7% | 2.5% |
| CEX-HPLC | Acidic peak: 25.1% | 20.6% | Acidic peak: 23.8% | 19.9% |
| | Main peak: 47.1% | 46.2% | Main peak: 49.1% | 50.8% |
| | Basic peak: 27.8% | 33.2% | Basic peak: 27.1% | 29.3% |
| icIEF | Acidic peak: 35.4% | 32.1% | Acidic peak: 35.1% | 30.8% |
| | Main peak: 40.9% | 41.3% | Main peak: 41.3% | 41.7% |
| | Basic peak: 23.6% | 26.6% | Basic peak: 23.6% | 27.6% |
| DAR | DAR value: 8.0 | 8.0 | DAR value: 8.0 | 8.0 |
| Free small | 0.000037% | 0.0010% | 0.00018% | 0.00050% |
| molecule payloads | | | | |
| Antigen binding | 105% | 103% | 96% | 97% |
| activity | | | | |
| Bioactivity | 102% | 101% | 93% | 103% |

TABLE 43
Stability results for key quality indicators of GMP Batch 2

| | GMP Batch 2 | | | |
|---|---|---|---|---|
| | Stock solution (5 ± 3 °C) | | Lyophilized powder (5 ± 3 °C) | |
| Items | 0 months | 6 months | 0 months | 6 months |
| SEC-HPLC | HMWS: 0.9% | 1.8% | HMWS: 0.9% | 1.1% |
| | Monomer: 96.8% | 96.5% | Monomer: 96.7% | 97.3% |
| | LMWS: 2.3% | 1.7% | LMWS: 2.3% | 1.6% |
| NR-CE-SDS | LC + HC: 97.0% | 97.5% | HC + LC: 97.3% | 97.2% |
| | Other peak: 3.0% | 2.5% | Other peak: 2.7% | 2.8% |
| CEX-HPLC | Acidic peak: 21.9% | 19.6% | Acidic peak: 22.3% | 20.0% |
| | Main peak: 46.4% | 46.9% | Main peak: 46.6% | 50.8% |
| | Basic peak: 31.7% | 33.5% | Basic peak: 31.2% | 29.2% |
| icIEF | Acidic peak: 33.0% | 31.9% | Acidic peak: 34.0% | 30.0% |
| | Main peak: 42.8% | 41.4% | Main peak: 42.8% | 43.4% |
| | Basic peak: 24.3% | 26.7% | Basic peak: 23.2% | 26.6% |
| DAR | DAR value: 8.0 | 8.0 | DAR value: 8.0 | 8.0 |
| Free small | 0.000048% | 0.0012% | 0.00078% | 0.00059% |
| molecule payloads | | | | |
| Antigen binding | 106% | 104% | 103% | 100% |
| activity | | | | |
| Bioactivity | 94% | 104% | 98% | 101% |

After replacing SWY2110-JSSW-001 with antibody-drug conjugates SWY2111-JSSW-001 and SWY2113-JSSW-001, stability studies at 5±3° C. were carried out separately on the stock solutions of the antibody-drug conjugates before lyophilization and the finished products of the antibody-drug conjugates after lyophilization using the same method described above. The study results and conclusions were substantially similar to those of SWY2110-JSSW-001, i.e., the quality of the product in the lyophilized state was more stable than that in the liquid state. The product in the liquid state showed significantly increased high molecular-weight fragments than the product in the lyophilized state. Moreover, the product in the liquid state was more prone to generating free small molecule payloads, with an obviously higher releasing rate compared to the lyophilized powder. The lyophilized dosage form was more conducive to the long-term storage of the product.

### Formulation Example 6

The antibody-drug conjugate (patritumab-JSSW-001) was respectively exchanged into 25 mM histidine-histidine hydrochloride buffer, citric acid-sodium citrate buffer and succinic acid-sodium hydroxide buffer at pH=5.0, 5.5, 6.0 and 6.5. The dilution was carried out to the extent that the protein concentration was about 20 mg/ml. Screening tests were conducted under high temperature (40±2° C., 75%±5% RH) and light exposure (5±3° C., white light: 5000±500 Lux, UV: 90 μW/cm²). Table 44 shows the appearance changes of the antibody-drug conjugate during the high temperature and light exposure tests. After 3 days of light exposure, the appearance of all 12 test groups changed significantly. The sodium citrate test groups and the succinate sodium test groups at pH=5.5, 6.0, and 6.5 showed phase separation or turbidity. The histidine test groups and the succinate sodium test group at pH=5.0 appeared slightly yellow. After 5 days at high temperature, except for the sodium citrate test groups at pH=6.0 and 6.5 which became turbid, the other test groups remained clear. The histidine test groups at pH=5.0, 5.5 and 6.0 and the succinate sodium test group at pH=5.0 were selected for the determination of SEC data after light exposure and high temperature tests. Table 45 shows the SEC changes for the pH and buffer salt type screening. After 5 days at high temperature, there was no significant difference in the SEC main peak among the four test groups, whereas after 3 days of light exposure, the SEC main peak of the histidine test group at pH=5.0 was significantly higher than that of the other test groups. Compared to high temperature, the antibody-drug conjugates are more sensitive to light. Histidine-histidine hydrochloride at pH=5.0 was an optimal buffer system.

### Detection Methods:

SEC-HPLC: According to Chinese Pharmacopoeia 2020 edition, Part IV, General Rule 0512 High Performance Liquid Chromatography, analysis and detection were performed using a chromatographic column packed with a gel suitable for separating proteins with a molecular weight of 10-500 kD. The proportions of the protein monomer peak, the high molecular weight substance (HMWS) peak and the low molecular weight substance (LMWS) peak were calculated according to the area normalization method.

### Detection Results:

TABLE 44
Appearance result

| | | | High |
|---|---|---|---|
| Buffer | 0 d | Light exposure, 3 d | temperature, 5 d |
| pH 5.0H | Clear | Slightly yellow | Clear |
| pH 5.5H | Clear | Slightly yellow | Clear |
| pH 6.0H | Clear | Slightly yellow | Clear |
| pH 6.5H | Clear | Turbid | Clear |
| pH 5.0C | Clear | Upper layer colorless, | Clear |
| | | lower layer yellow | |
| pH 5.5C | Clear | Upper layer colorless, | Clear |
| | | lower layer yellow | |
| pH 6.0C | Clear | Upper layer turbid, | Slightly turbid |
| | | lower layer yellow | |
| pH 6.5C | Clear | Upper layer turbid, | Turbid |
| | | lower layer yellow | |
| pH 5.0S | Clear | Slightly yellow | Clear |
| pH 5.5S | Clear | Turbid | Clear |
| pH 6.0S | Clear | Turbid | Clear |
| pH 6.5S | Clear | Turbid | Clear |

Note:
H: histidine, C: sodium citrate, S: sodium succinate

TABLE 45
SEC-HPLC detection result

| | SEC-HPLC purity (%) | | | | | | | | |
|---|---|---|---|---|---|---|---|---|---|
| | 0 d | | | 5 °C, light exposure, 3 d | | | 40 °C, high temperature, 5 d | | |
| | | Main | | | Main | | | Main | |
| | Aggregate | peak | Fragment | Aggregate | peak | Fragment | Aggregate | peak | Fragment |
| Buffer | (%) | (%) | (%) | (%) | (%) | (%) | (%) | (%) | (%) |
| pH 5.0H | 1.11 | 98.17 | 0.72 | 28.96 | 70.15 | 0.89 | 3.29 | 95.81 | 0.9 |
| pH 5.5H | 1.41 | 97.89 | 0.7 | 42.91 | 56.22 | 0.87 | 3.26 | 96.02 | 0.72 |
| pH 6.0H | 1.37 | 97.99 | 0.64 | 55.91 | 42.92 | 1.17 | 7.51 | 91.44 | 1.05 |
| pH 5.0S | 1.59 | 97.69 | 0.72 | 32.95 | 63.63 | 3.42 | 3.94 | 95.43 | 0.63 |

Note:
H: histidine,
S: sodium succinate

### Formulation Example 7

The antibody-drug conjugate (patritumab-JSSW-001) was respectively exchanged into different concentrations (15 mM, 25 mM, 35 mM) of histidine-histidine hydrochloride buffers containing 6% sucrose at pH=5.0. The dilution was carried out to the extent that the protein concentration was about 20 mg/ml. Screening tests were conducted under high temperature and light exposure conditions. SEC and CEX were determined to screen for the optimal histidine concentration. Table 46 shows the DSC data for screening the histidine concentrations. Table 47 shows the SEC data for screening the histidine concentrations. In the light exposure test, the SEC main peak decreased significantly as the histidine concentration increased; in the high temperature test, the SEC main peak decreased slightly as the histidine concentration increased; due to the relatively strong photosensitivity of the antibody-drug conjugate, the CEX chromatogram from the light exposure test could no longer distinguish the acidic peak, main peak, and basic peak, so only the CEX data from the high temperature test were statistically analyzed. Table 48 shows the CEX main peak data from the high temperature test for screening the histidine concentrations. The results indicated that histidine concentrations had little effect on the CEX main peak. Based on the SEC and CEX data, 15 mM was an optimal concentration for histidine.

### Detection Method

DSC: A sample to be tested was diluted with the same buffer therein to a protein concentration of 1 mg/mL. Then, 350 μL each of the test sample and the corresponding buffer were added to the designated wells of a 96-well plate. The 96-well plate was placed into a sample holder, and the temperature of the sample holder was set to 15° C. Instrument parameters were set as follows: initial temperature: 20° C., final temperature: 100° C., and heating rate: 90° C./min.

SEC-HPLC: Detection method was identical to that in Formulation Example 6.

CEX-HPLC: According to Chinese Pharmacopoeia 2020 edition, Part IV, General Rule 0512 High Performance Liquid Chromatography, analysis and detection were performed using a cation exchange column as the chromatographic column. The proportions of the acidic peak, main peak, and basic peak of the test sample were calculated according to the area normalization method.

### Detection Results:

TABLE 46
DSC detection results

| Histidine concentration | Tₒₙₛₑₜ(°C) | Tm₁(°C) | Tm₂(°C) |
|---|---|---|---|
| 15 mM | 51.3 | 58.3 | 78.5 |
| 25 mM | 50.2 | 58.7 | 77.2 |
| 35 mM | 49.7 | 60.7 | 77.2 |

TABLE 47
SEC-HPLC detection result

| | 0 d SEC | | | Light exposure 5 d SEC | | | Light exposure 10 d SEC | | |
|---|---|---|---|---|---|---|---|---|---|
| | | Main | | | Main | | | Main | |
| Histidine | Aggregate | peak | Fragment | Aggregate | peak | Fragment | Aggregate | peak | Fragment |
| concentration | (%) | (%) | (%) | (%) | (%) | (%) | (%) | (%) | (%) |
| 15 mM | 0.96 | 98.48 | 0.56 | 12.50 | 85.21 | 2.29 | 13.14 | 84.08 | 2.78 |
| 25 mM | 1.03 | 98.39 | 0.58 | 14.89 | 83.14 | 1.97 | 15.61 | 81.89 | 2.50 |
| 35 mM | 1.09 | 98.33 | 0.58 | 18.63 | 79.33 | 2.04 | 19.43 | 78.00 | 2.57 |
| | | | | High temperature 7 d | | | High temperature 14 d | | |
| | 0 d SEC | | | SEC | | | SEC | | |
| | | Main | | | Main | | | Main | |
| Histidine | Aggregate | peak | Fragment | Aggregate | peak | Fragment | Aggregate | peak | Fragment |
| concentration | (%) | (%) | (%) | (%) | (%) | (%) | (%) | (%) | (%) |
| 15 mM | 0.96 | 98.48 | 0.56 | 1.37 | 97.61 | 1.02 | 1.55 | 97.24 | 1.21 |
| 25 mM | 1.03 | 98.39 | 0.58 | 1.81 | 97.10 | 1.09 | 2.58 | 96.05 | 1.37 |
| 35 mM | 1.09 | 98.33 | 0.58 | 2.08 | 96.82 | 1.10 | 2.97 | 95.67 | 1.36 |

TABLE 48
CEX-HPLC detection result

| | | | | High temperature 7 d | | | High temperature 14 d | | |
|---|---|---|---|---|---|---|---|---|---|
| | 0 d CEX | | | CEX | | | CEX | | |
| | Acidic | Main | Basic | Acidic | Main | Basic | Acidic | Main | Basic |
| Histidine | peak | peak | peak | peak | peak | peak | peak | peak | peak |
| concentration | (%) | (%) | (%) | (%) | (%) | (%) | (%) | (%) | (%) |
| 15 mM | 38.35 | 58.3 | 3.35 | 44.26 | 52.13 | 3.61 | 51.64 | 44.42 | 3.94 |
| 25 mM | 37.79 | 58.97 | 3.24 | 44.15 | 51.94 | 3.91 | 51.62 | 43.59 | 4.79 |
| 35 mM | 38.05 | 57.07 | 4.88 | 43.19 | 53.4 | 3.41 | 49.25 | 46.03 | 4.72 |

### Formulation Example 8

15 mM histidine-histidine hydrochloride buffer at pH=5.0 was prepared, and sucrose or trehalose was added separately at each of 4%, 6%, 8%, and 10%. The antibody-drug conjugate (patritumab-JSSW-001) was exchanged through ultrafiltration into the above 8 buffers, with the protein concentration diluted to approximately 20 mg/ml. After filtering through a 0.22 μm microporous filter membrane, the thermal stability of the antibody-drug conjugate was detected by DSC, and stability tests were performed under high temperature and light exposure.

Table 49 shows the thermal stability data of the antibody-drug conjugate in buffer systems with different concentrations of sucrose and trehalose. The type and addition amount of the stabilizer had no significant effect on the thermal denaturation onset temperature Tonset (° C.), while the test group with the highest Tml value was the 8% sucrose group. Table 50 shows the effect of high temperature and light exposure on the SEC monomer of the antibody-drug conjugate in buffer systems with different types and concentrations of stabilizers. After 14 days at high temperature, the SEC monomer of the 4% test group was significantly lower than that of the other test groups. Under the same saccharide concentration, there was no significant difference in SEC between the sucrose and trehalose groups in the high temperature test. Table 51 shows the effect of high temperature on the CEX main peak of the antibody-drug conjugate in buffer systems with different types and concentrations of stabilizers. After the high temperature test, there was no significant difference in the CEX main peak among the 8 test groups. In summary, 8% sucrose or trehalose could be added as a stabilizer. Considering that the cost of trehalose was higher than that of sucrose, 8% sucrose was selected for this product.

TABLE 49
DSC detection results

| Stabilizer | Tₒₙₛₑₜ(°C) | Tm₁(°C) | Tm₂(°C) |
|---|---|---|---|
| 4% sucrose | 51.3 | 59.0 | 77.8 |
| 6% sucrose | 51.7 | 59.4 | 78.0 |
| 8% sucrose | 51.5 | 61.4 | 78.2 |
| 10% sucrose | 52.5 | 59.0 | 78.7 |
| 4% trehalose | 50.8 | 59.0 | 77.8 |
| 6% trehalose | 51.1 | 59.2 | 78.0 |
| 8% trehalose | 52.5 | 58.4 | 78.6 |
| 10% trehalose | 51.7 | 60.0 | 78.5 |

TABLE 50
SEC detection result

| | | | | High temperature | | | High temperature | | |
|---|---|---|---|---|---|---|---|---|---|
| | 0 d SEC | | | 7 d SEC | | | 14 d SEC | | |
| | | Main | | | Main | | | Main | |
| | Aggregate | peak | Fragment | Aggregate | peak | Fragment | Aggregate | peak | Fragment |
| Stabilizer | (%) | (%) | (%) | (%) | (%) | (%) | (%) | (%) | (%) |
| 4% sucrose | 1.00 | 98.22 | 0.78 | 1.31 | 97.50 | 1.19 | 2.26 | 95.87 | 1.87 |
| 6% sucrose | 1.02 | 98.26 | 0.72 | 1.18 | 97.63 | 1.19 | 2.04 | 96.18 | 1.78 |
| 8% sucrose | 0.99 | 98.26 | 0.75 | 1.13 | 97.60 | 1.27 | 1.97 | 96.26 | 1.77 |
| 10% sucrose | 1.01 | 98.27 | 0.72 | 1.18 | 97.60 | 1.22 | 1.84 | 96.41 | 1.75 |
| 4% trehalose | 1.08 | 98.11 | 0.81 | 1.32 | 97.51 | 1.17 | 2.38 | 95.82 | 1.80 |
| 6% trehalose | 1.05 | 98.18 | 0.77 | 1.21 | 97.71 | 1.08 | 2.07 | 96.24 | 1.69 |
| 8% trehalose | 1.06 | 98.12 | 0.82 | 1.29 | 97.51 | 1.20 | 2.04 | 96.29 | 1.67 |
| 10% trehalose | 1.06 | 98.08 | 0.86 | 1.24 | 97.58 | 1.18 | 1.98 | 96.23 | 1.79 |
| | | | | Light exposure | | | Light exposure | | |
| | 0 d SEC | | | 5 d SEC | | | 10 d SEC | | |
| | | Main | | | Main | | | Main | |
| | Aggregate | peak | Fragment | Aggregate | peak | Fragment | Aggregate | peak | Fragment |
| Stabilizer | (%) | (%) | (%) | (%) | (%) | (%) | (%) | (%) | (%) |
| 4% sucrose | 1.00 | 98.22 | 0.78 | 13.05 | 84.07 | 2.89 | 17.27 | 78.62 | 4.12 |
| 6% sucrose | 1.02 | 98.26 | 0.72 | 12.02 | 85.23 | 2.76 | 15.49 | 80.53 | 3.99 |
| 8% sucrose | 0.99 | 98.26 | 0.75 | 11.16 | 86.10 | 2.75 | 14.36 | 81.75 | 3.89 |
| 10% sucrose | 1.01 | 98.27 | 0.72 | 11.18 | 86.11 | 2.71 | 14.32 | 81.76 | 3.93 |
| 4% trehalose | 1.08 | 98.11 | 0.81 | 13.93 | 83.40 | 2.67 | 17.86 | 78.24 | 3.90 |
| 6% trehalose | 1.05 | 98.18 | 0.77 | 11.79 | 85.52 | 2.70 | 14.96 | 81.18 | 3.87 |
| 8% trehalose | 1.06 | 98.12 | 0.82 | 12.46 | 84.81 | 2.74 | 15.97 | 80.13 | 3.91 |
| 10% trehalose | 1.06 | 98.08 | 0.86 | 11.61 | 85.78 | 2.61 | 14.91 | 81.28 | 3.81 |

TABLE 51
CEX detection result

| | | | | High temperature | | | High temperature | | |
|---|---|---|---|---|---|---|---|---|---|
| | 0 d | | | 7 d CEX | | | 14 d CEX | | |
| | Acidic | Main | Basic | Acidic | Main | Basic | Acidic | Main | Basic |
| | peak | peak | peak | peak | peak | peak | peak | peak | peak |
| Stabilizer | (%) | (%) | (%) | (%) | (%) | (%) | (%) | (%) | (%) |
| 4% sucrose | 35.03 | 61.54 | 3.43 | 47.22 | 51.18 | 1.6 | 52.09 | 45.85 | 2.06 |
| 6% sucrose | 37.48 | 59.32 | 3.2 | 46.92 | 51.74 | 1.34 | 53.74 | 44.28 | 1.98 |
| 8% sucrose | 36.05 | 59.81 | 4.14 | 47.09 | 51.37 | 1.54 | 53.09 | 45.06 | 1.85 |
| 10% sucrose | 37.62 | 59.46 | 2.92 | 47.24 | 51.11 | 1.65 | 53.16 | 44.91 | 1.93 |
| 4% trehalose | 36.71 | 59.66 | 3.63 | 45.85 | 52.37 | 1.78 | 53.36 | 44.44 | 2.20 |
| 6% trehalose | 37.82 | 58.85 | 3.33 | 46.34 | 51.89 | 1.77 | 52.68 | 45.52 | 1.80 |
| 8% trehalose | 37.97 | 58.52 | 3.51 | 45.37 | 52.95 | 1.68 | 52.94 | 45.29 | 1.77 |
| 10% trehalose | 35.85 | 60.6 | 3.55 | 46.52 | 51.74 | 1.74 | 53.47 | 44.81 | 1.72 |

### Formulation Example 9

15 mM histidine-histidine hydrochloride buffer at pH=5.0 was prepared, and sucrose was added at 8% o. The antibody-drug conjugate (patritumab-JSSW-001) sample was exchanged into the above solution through ultrafiltration. Different concentrations of polysorbate 80 were added to prepare samples with an antibody-drug conjugate concentration of 20 mg/ml. The samples were dispensed into injection vials, and a stability study was conducted under freezing-thawing conditions (frozen at −20° C. and thawed at room temperature for a total of 6 cycles) and shaking (5° C., 200 rpm for 10 days). The changes in subvisible particles in the samples under different conditions were detected using a microflow imaging instrument (Flowcam). Table 52 shows the effect of freezing-thawing and shaking on insoluble particles in samples with different addition amounts of polysorbate 80. The results showed that the addition of 0.005% polysorbate 80 could significantly inhibit particle formation, while 0.02% polysorbate 80 was preferred for preventing aggregate formation of the antibody-drug conjugate during long-term storage.

TABLE 52
Effect of freezing-thawing and shaking on insoluble particles
in samples with different addition amounts of polysorbate 80

| | Particle number (P/mL) | | | | | |
|---|---|---|---|---|---|---|
| | | | 6 cycles of freezing- | | | |
| | T0 | | thawing | | shaking 10 d | |
| PS80 | NSP(2- | | NSP(2- | | NSP(2- | |
| concentration | 10 μm) | NSP(≥10 μm) | 10 μm) | NSP(≥10 μm) | 10 μm) | NSP(≥10 μm) |
| 0% | 197 | 15 | 2306 | 254 | 5381 | 580 |
| 0.005% | 8 | 1 | 29 | 0 | 438 | 2 |
| 0.02% | 35 | 2 | 42 | 2 | 556 | 0 |
| 0.04% | 66 | 2 | 88 | 1 | 1225 | 0 |
| 0.06% | 38 | 1 | 86 | 0 | 416 | 0 |

### Formulation Example 10: Lyophilization Process Development

A formula of 20 mg/ml of the antibody-drug conjugate (patritumab-JSSW-001), 15 mmol/L of histidine-histidine hydrochloride, 0.02% of polysorbate 80, and 8% of sucrose at pH=5.0 was selected as base formula. The glass transition temperature and collapse temperature of a sample having this base formula were detected. Low-temperature DSC detection showed the glass transition temperature of the sample was −28.79° C., and the lyophilization microscopy detection showed the collapse temperature of the sample was −28.1° C.

The pre-freezing temperature was pre-determined to −40° C. Before the pre-freezing temperature reached −40° C., the shelf temperature was first brought to 5° C. over 30 minutes and maintained at 5° C. for another 30 minutes to ensure that all samples could start cooling from 5° C. Pre-freezing was performed at three cooling rates: 0.5° C./min, 2° C./min, and 1° C./min. The lyophilized powders obtained with different pre-freezing cooling rates were all white, cake-like, and loose solids. After adding 5.0 ml of water for injection, all three batches of lyophilized powders could be reconstituted to produce a clear liquid within 3 minutes. Table 53 shows the moisture content of lyophilized powders containing the antibody-drug conjugate from 5 different positions. The lyophilized powder obtained at the cooling rate of 0.5° C./min had the lowest average moisture content.

TABLE 53
Comparison of moisture contents of lyophilized powders
at different cooling rates of pre-freezing

| | Measured moisture content (%) | | |
|---|---|---|---|
| | Cooling rate: | Cooling rate: | Cooling rate: |
| Sample position No. | 0.5 °C/min | 1 °C/min | 2 °C/min |
| A | 1.19 | 1.15 | 1.13 |
| B | 1.25 | 1.37 | 1.34 |
| C | 1.15 | 1.43 | 1.29 |
| D | 1.21 | 1.37 | 1.39 |
| E | 1.18 | 1.15 | 1.09 |
| Average | 1.19 | 1.28 | 1.24 |

Note:
C was the center position of the shelf, A was a corner of the shelf near the door, E was the diagonal corner of A near the isolation valve, B and D were the midpoints of AC and CE, respectively.

Primary drying: The shelf temperature was set above the allowable collapse temperature of the product, ensuring that the temperature of the non-sublimed portion of the product did not exceed its collapse temperature. With pre-freezing performed at a cooling rate of 0.5° C./min and secondary drying conducted at 35° C. and 0.2 mbar for 900 minutes, lyophilization trials were carried out using five different shelf temperatures (-15° C., −12° C., −8° C., −5° C., and 0° C.) for primary drying. The temperature curve of the sample with the probe was observed during the early stage of primary drying (before the temperature rise inflection point appears), in which the temperature of the frozen portion of the sample contacted by the probe was in the range from −28 to −30° C., none exceeding the collapse temperature (Tc: −28.1° C.). All five batches ultimately yielded white loose lyophilized powder without severe collapse. After adding 5.0 ml of water for injection, all five batches of lyophilized powders could be reconstituted to produce a clear liquid within 3 minutes. Table 54 shows the primary drying time and moisture content of the lyophilized powder under different primary drying conditions. The primary drying time shortened as the set value of the shelf temperature increased, with the shortest time when the shelf temperature was set to 0° C. Under the same secondary drying condition (35° C., 0.2 mbar, 900 min), moisture contents of lyophilized powders obtained by different primary drying processes were compared. The moisture content of the lyophilized powder obtained through primary drying at 0° C. (1.52%) was significantly higher than that at −5° C. (1.26%). Therefore, the shelf temperature was set to −5° C. for primary drying. At the primary drying temperature set at −5° C., the lyophilization effects at 0.15 mbar, 0.20 mbar, and 0.25 mbar were compared. The batch obtained under the vacuum value of the primary drying set at 0.25 mbar had the lowest moisture content. Therefore, the vacuum value of the lyophilization chamber of the primary drying was set to 0.25 mbar.

TABLE 54
Primary drying time and moisture content of the lyophilized
powder under different primary drying conditions

| | Primary drying | Moisture |
|---|---|---|
| Primary drying condition | time(min) | content(%) |
| −15 °C, 0.2 mbar | 2500 | 1.19 |
| −12 °C, 0.2 mbar | 2300 | 1.26 |
| −8 °C, 0.2 mbar | 2000 | 1.17 |
| −5 °C, 0.2 mbar | 1800 | 1.26 |
| 0 °C, 0.2 mbar | 1600 | 1.52 |
| −5 °C, 0.15 mbar | 1800 | 1.44 |
| −5 °C, 0.25 mbar | 1800 | 1.22 |

Secondary drying: The shelf temperature was set according to the thermal stability of the product. The thermal denaturation onset temperature (Tₒₙₛₑₜ) of the stock solution of the antibody-drug conjugate determined by differential scanning calorimetry was around 50° C., so the set value of the shelf temperature for secondary drying needed to be below 50° C. The pre-freezing adopted a cooling rate of 0.5° C./min, the primary drying adopted −5° C., and the vacuum value was 0.25 mbar. Table 55 shows the comparison data of the moisture and purity of lyophilized powders obtained by two secondary drying methods. Compared to the stock solution, the purity of the antibody-drug conjugate in the lyophilized powder obtained at both secondary drying temperatures showed no significant difference, but the lyophilized powder from secondary drying at 35° C. for 900 min had a lower moisture content. In summary, the secondary drying process was determined to be performed at 35° C. under the vacuum value of 0.25 mbar.

TABLE 55
Comparison data of the moisture and purity of lyophilized
powders obtained by two secondary drying methods

| | | | | | CEX | | | |
|---|---|---|---|---|---|---|---|---|
| | Secondary | SEC | | | Acidic | Main | Basic | |
| | drying | Aggregate | Monomer | Fragment | peak | peak | peak | Moisture |
| Batch number | conditions | (%) | (%) | (%) | (%) | (%) | (%) | (%) |
| DS113 (Stock | N/A | 0.61 | 99.39 | 0.00 | 27.46 | 66.41 | 6.13 | N/A |
| solution) | | | | | | | | |
| DP116 | 30 °C, | 0.66 | 99.34 | 0.00 | 29.09 | 64.77 | 6.14 | 1.52 |
| (Reconstitution | 0.25 mbar, | | | | | | | |
| solution of | 1200 min | | | | | | | |
| lyophilized | | | | | | | | |
| powder) | | | | | | | | |
| DP129 | 35 °C, | 0.66 | 99.34 | 0.00 | 27.28 | 66.87 | 5.85 | 1.34 |
| (Reconstitution | 0.25 mbar, | | | | | | | |
| solution of | 900 min | | | | | | | |
| lyophilized | | | | | | | | |
| powder) | | | | | | | | |

### Lyophilization Pilot-Scale Process Transfer:

The lyophilization pilot-scale process transfer determined the primary drying time based on the product temperature curve and the pressure rise test (the criterion for the pressure rise at the end of primary drying was <0.01 mbar/min). The secondary drying conditions were consistent with those in the lab-scale trial. A total of four batches of lyophilization pilot-scale process transfers were conducted. For the first batch of pilot-scale process transfer, the lyophilization formula that was optimized during the lab-scale trial was used. The lyophilized powders located in the middle of the shelf showed severe pits on the sidewall, while this phenomenon was less severe at the edges of the shelf. For the second batch of pilot-scale process transfer, the shelf temperature was set to −20° C. during primary drying. However, the sidewall pits of the lyophilized powders located in the middle of the shelf showed no significant improvement. For the third batch of pilot-scale process transfer, an annealing step at −10° C. was introduced during pre-freezing to allow the recrystallization of the portions that did not fully crystallize during pre-freezing, resulting in a more uniform crystal form and size distribution and reducing the resistance of moisture escaping from the dried layer; the sidewall pits of the lyophilized powder in the middle of the shelf were slightly improved. For the fourth batch of pilot-scale process transfer, based on the introduction of the annealing step at −10° C. during pre-freezing, the vacuum value of primary drying was reduced to 0.06 mbar to enhance the escape ability of water vapor after sublimation, reduce the accumulation of moisture in the dried layer, and avoid the thawing of the dried layer due to accumulated moisture; the pitting phenomenon in the samples located in the middle of the shelf was significantly improved. Table 56 shows the summarized data of the lyophilization pilot-scale process transfer. Table 57 shows the purity comparison data between a reconstitution solution of lyophilized powder from the pilot-scale process transfer and the stock solution before lyophilization. The reconstitution solution of lyophilized powder and the stock solution before lyophilization showed no significant change in the purity.

TABLE 56
Summarized data of the lyophilization pilot-scale process transfer

| | Pre-freezing | | | | Lyophilized |
|---|---|---|---|---|---|
| | cooling rate | Primary | Secondary | Moisture | powder |
| Batch | (°C./min) | drying condition | drying condition | (%) | morphology |
| 1 | 0.5 | −5 °C, 0.25 mbar | 35 °C, | 1.22 | Severe pits |
| 2 | 0.5 | −20 °C, 0.25 mbar | 0.25 mbar | 0.77 | Severe pits |
| 3 | 0.5 (−10 °C | −5 °C, 0.25 mbar | | 0.90 | Improved |
| | annealing) | | | | pitting |
| | | | | | phenomenon |
| 4 | 0.5 (−10 °C | −5 °C, 0.06 mbar | | 0.69 | No significant |
| | annealing) | | | | pitting |

TABLE 57
Purity comparison data between reconstitution solution of lyophilized powder from
the pilot-scale process transfer and stock solution before lyophilization

| | SEC | | | CEX | | | |
|---|---|---|---|---|---|---|---|
| | Aggregate | Monomer | Fragment | Acidic | Main | Basic | |
| Batch | (%) | (%) | (%) | peak (%) | peak (%) | peak (%) | DAR |
| Stock solution | 0.65 | 99.35 | 0 | 26.81 | 66.77 | 6.42 | 8 |
| before | | | | | | | |
| lyophilization | | | | | | | |
| 1 | 0.71 | 99.29 | 0 | 27.51 | 65.84 | 6.65 | 8 |
| 2 | 0.70 | 99.30 | 0 | 29.03 | 64.56 | 6.41 | 8 |
| 3 | 0.67 | 99.33 | 0 | 27.77 | 65.94 | 6.29 | 8 |
| 4 | 0.67 | 99.30 | 0 | 28.18 | 65.73 | 6.09 | 8 |

Detection methods: SEC and CEX were identical to those in Formulation Example 7.

DAR distribution: According to Chinese Pharmacopoeia 2020 edition, Part IV, General Rule 0512 High Performance Liquid Chromatography, analysis and detection were performed using a hydrophobic chromatographic column (e.g., TSKgel Butyl-NPR, 4.6×100 mm or other suitable columns) as the chromatographic column. The mobile phase A included 10 M ammonium sulfate and 25 mM phosphate buffer (pH=7.0), and the mobile phase B included 25% isopropanol and 75% 25 mM phosphate buffer (pH=7.0). The column temperature was 30° C., the flow rate was 0.5 ml/min, and the detection wavelength was 280 nm. An appropriate amount of the test sample solution was injected into the liquid chromatograph for gradient elution. The DAR value was calculated.

### Formulation Example 11

Stability studies at 5±3° C. were carried out separately on the stock solution of the antibody-drug conjugate (patritumab-JSSW-001) before lyophilization and the finished product of the antibody-drug conjugate (patritumab-JSSW-001) after lyophilization. The study results showed that the quality of the products in the lyophilized state was more stable than that in the liquid state. The product in the liquid state showed significantly increased high molecular-weight fragments than the product in the lyophilized state and had increased acidic peak contents of charge variants. Moreover, the product the liquid state was more prone to generating free small molecule payloads, with an obviously higher releasing rate compared to the lyophilized powder. The lyophilized dosage form was more conducive to the long-term storage of the product.

### Detection Methods:

SEC-HPLC: Detection method was identical to that in Formulation Example 6.

NR-CE-SDS: Detection method was identical to that in Formulation Example 5.

CZE: Analysis and detection were performed by using the capillary zone electrophoresis separation mode with reference to capillary electrophoresis (Chinese Pharmacopoeia 2020 edition, Part IV, General Rule 0542). The components in the test sample were separated according to their respective electrophoretic and electroosmotic velocities. After separation, the proportions of the acidic peak, main peak, and basic peak were calculated according to the peak area normalization method.

DAR distribution: Detection method was identical to that in Formulation Example 10.

Free small molecule payload content: According to Chinese Pharmacopoeia 2020 edition, Part IV, General Rule 0512 High Performance Liquid Chromatography and 0431 Mass Spectrometry, analysis and detection were performed using a chromatographic column packed with an octadecylsilyl silica gel (e.g., Waters ACQUITY UPLC® BEH-C18, 2.1×50 mm, particle size 1.7 μm or other suitable chromatographic columns). The mobile phase A was an aqueous solution containing 0.10% FA, and the mobile phase B was an acetonitrile solution containing 0.1% FA. The column temperature was 40° C., and the flow rate was 0.4 ml/min. 50 μL of the test sample (10 mg/mL) was taken, and an internal standard working solution was added. The mixture was precipitated with acetonitrile in the ratio of 1:3, placed in a −20° C. ice bath for 30 minutes, and then centrifuged. The supernatant was taken and loaded onto the column. Different concentrations of small molecule payloads were used as reference standards and loaded onto the column. After gradient elution by liquid chromatography, the sample was sent to the mass spectrometer detector. After setting parameters such as capillary and cone voltages, ion source temperature, collision energy, and nebulizer flow rate, the analysis was performed. The free small molecule payload content in the test sample was calculated according to the standard curve.

Free small molecule payloads content(%)=Detection concentration of free small molecule payloads/ADC concentration×100%

TABLE 58
Stability results for key quality indicators of GMP Batch 1

| | GMP Batch 1 | | | |
|---|---|---|---|---|
| | Stock solution (5 ± 3 °C) | | Lyophilized powder (5 ± 3 °C) | |
| Items | 0 months | 6 months | 0 months | 6 months |
| SEC-HPLC | HMWS: 0.4% | 1.1% | HMWS: 0.4% | 0.4% |
| | Monomer: 99.6% | 98.9% | Monomer: 99.6% | 99.6% |
| NR-CE-SDS | LC + HC: 96.5% | 95.7% | HC + LC: 96.4% | 96.4% |
| | Other peak: 3.4% | 4.3% | Other peak: 3.6% | 3.6% |
| CZE | Acidic peak: 19.2% | 25.1% | Acidic peak: 19.7% | 20.8% |
| | Main peak: 75.1% | 68.4% | Main peak: 74.8% | 73.4% |
| | Basic peak: 5.7% | 6.5% | Basic peak: 5.5% | 5.9% |
| DAR | DAR value: 8.0 | 8.0 | DAR value: 8.0 | 8.0 |
| Free small molecule | 0.000038% | 0.00076% | 0.00012% | 0.00023% |
| payloads | | | | |
| Antigen binding | 96% | 94% | 93% | 99% |
| activity | | | | |
| Bioactivity | 103% | 105% | 107% | 106% |

TABLE 59
Stability results for key quality indicators of GMP Batch 2

| | GMP Batch 2 | | | |
|---|---|---|---|---|
| | Stock solution (5 ± 3 °C) | | Lyophilized powder (5 ± 3 °C) | |
| Items | 0 months | 6 months | 0 months | 6 months |
| SEC-HPLC | HMWS: 0.5% | 1.0% | HMWS: 0.6% | 0.4% |
| | Monomer: 99.5% | 99.0% | Monomer: 99.4% | 99.6% |
| NR-CE-SDS | LC + HC: 95.9% | 95.1% | HC + LC: 95.6% | 96.3% |
| | Other peak: 4.1% | 5.0% | Other peak: 4.4% | 3.7% |
| CZE | Acidic peak: 18.6% | 25.8% | Acidic peak: 18.1% | 18.9% |
| | Main peak: 75.0% | 67.4% | Main peak: 75.4% | 74.6% |
| | Basic peak: 6.4% | 6.7% | Basic peak: 6.4% | 6.5% |
| DAR | DAR value: 8.0 | 8.0 | DAR value: 8.0 | 8.0 |
| Free small | 0.000031% | 0.00067% | 0.00018% | 0.00025% |
| molecule payloads | | | | |
| Antigen binding | 100% | 95% | 105% | 92% |
| activity | | | | |
| Bioactivity | 102% | 104% | 105% | 106% |

### Formulation Example 12.1: Preparation Example of Formulation

(1) Buffer exchanging through ultrafiltration: A solution of 25 mM histidine-histidine hydrochloride and 9% trehalose at pH=5.0 was prepared. The buffer exchanging was performed by using a 30 kD ultrafiltration membrane pack. The antibody-drug conjugate was exchanged into the solution of 25 mM histidine-histidine hydrochloride and 9% trehalose at pH=5.0. The exchanging factor for the buffer exchanging was no less than 100-fold. The concentration of the ADC in the exchanging solution was controlled to be no less than 1.2 times the target concentration.
(2) Preparation of semi-finished product: According to the amounts in the formula, the corresponding amount of polysorbate 80 was added to the ultrafiltration exchanging solution. The ADC was diluted to the target concentration with a solution containing 25 mM histidine-histidine hydrochloride and 9% trehalose at pH=5.0. After stirring and mixing uniformly, a target solution containing 10 mg/ml ADC, 25 mM histidine-histidine hydrochloride, 9% trehalose, and 0.03% polysorbate 80 was obtained. After filtering with a two-stage 0.22 μm sterilization filter, an aqueous solution of the SWY2110-JSSW001 formulation was obtained, which contained about 10 mg/ml of the antibody-drug conjugate SWY2110-JSSW-001, about 0.33 mg/ml of histidine, about 4.79 mg/ml of histidine hydrochloride, about 0.3 mg/ml of polysorbate 80, about 90 mg/ml of trehalose, and the balance of water.
(3) Filling and lyophilization: According to the filling volume of the product, the filling of sample products and the semi-stoppering were performed on a fully automatic filling line. The semi-stoppered products were transferred via an automatic conveyor into a lyophilizer for freeze-drying. The conditions were controlled as follows: the pre-freezing was performed at −45° C. for not less than 180 minutes; the primary drying was performed at −20° C. and 0.20 mbar for not less than 4000 minutes; the secondary drying was performed at 30° C. and 0.20 mbar for not less than 1200 minutes. After the lyophilization was completed, the stoppering was performed in the lyophilizer, then the product was transferred to the capping station via an automatic conveyor to complete the capping. After passing the lamp inspection, the final product, SWY2110-JSSW001 formulation, was obtained, which contained about 10 parts by weight of antibody-drug conjugate SWY2110-JSSW-001, about 0.33 parts by weight of histidine, about 4.79 parts by weight of histidine hydrochloride, about 0.3 parts by weight of polysorbate 80, and about 90 parts by weight of trehalose.

### Formulation Example 12.2: Preparation Example of Formulation

(1) Buffer exchanging through ultrafiltration: A solution of 15 mM histidine-histidine hydrochloride and 8% sucrose at pH=5.0 was prepared. The buffer exchanging was performed by using a 30 kD ultrafiltration membrane pack. The antibody-drug conjugate was exchanged into the solution of 15 mM histidine-histidine hydrochloride and 8% sucrose at pH=5.0. The exchanging factor for the buffer exchanging was no less than 100-fold. The concentration of the ADC in the exchanging solution was controlled to be no less than 1.2 times the target concentration.
(2) Preparation of semi-finished product: According to the amounts in the formula, the corresponding amount of polysorbate 80 was added to the ultrafiltration exchanging solution. The ADC was diluted to the target concentration with a solution containing 15 mM histidine-histidine hydrochloride and 8% sucrose at pH=5.0. After stirring and mixing uniformly, a target solution containing 20 mg/ml ADC, 15 mM histidine-histidine hydrochloride, 8% sucrose, and 0.02% polysorbate 80 was obtained. After filtering with a two-stage 0.22 μm sterilization filter, an aqueous solution of the Patritumab-JSSW001 formulation was obtained, which contained about 20 mg/ml of patritumab-JSSW-001 antibody-drug conjugate, about 0.21 mg/ml of histidine, about 2.86 mg/ml of histidine hydrochloride, about 0.2 mg/ml of polysorbate 80, about 80 mg/ml of sucrose, and the balance of water.
(3) Filling and lyophilization: According to the filling volume of the product, the filling of sample products and the semi-stoppering were performed on a fully automatic filling line. The semi-stoppered products were transferred via an automatic conveyor into a lyophilizer for freeze-drying. The conditions were controlled as follows: the pre-freezing was performed at −40° C. for not less than 180 minutes; the primary drying was performed at −5° C. and 0.06 mbar for not less than 3000 minutes; the secondary drying was performed at 35° C. and 0.25 mbar for not less than 900 minutes. After the lyophilization was completed, the stoppering was performed in the lyophilizer, then the product was transferred to the capping station via an automatic conveyor to complete the capping. After passing the lamp inspection, the final product, patritumab-JSSW001 formulation, was obtained, which contained about 20 parts by weight of antibody-drug conjugate patritumab-JSSW-001, about 0.21 parts by weight of histidine, about 2.86 parts by weight of histidine hydrochloride, about 0.2 parts by weight of polysorbate 80, and about 80 parts by weight of sucrose.

### Formulation Example 13: Bioactivity Assay

An aqueous solution of the SWY2110-JSSW001 formulation and an aqueous solution of the Patritumab-JSSW001 formulation, respectively obtained in step (2) of Formulation Examples 12.1 and 12.2, were used to determine the biological activities of two formulations according to the method for determining biological activity described in the preceding Biological Examples section. The results indicated that the biological activities of the two formulations were comparable to those of the standalone ADCs.

1-12. (canceled)

13. A pharmaceutical composition containing an antibody-drug conjugate represented by formula I: 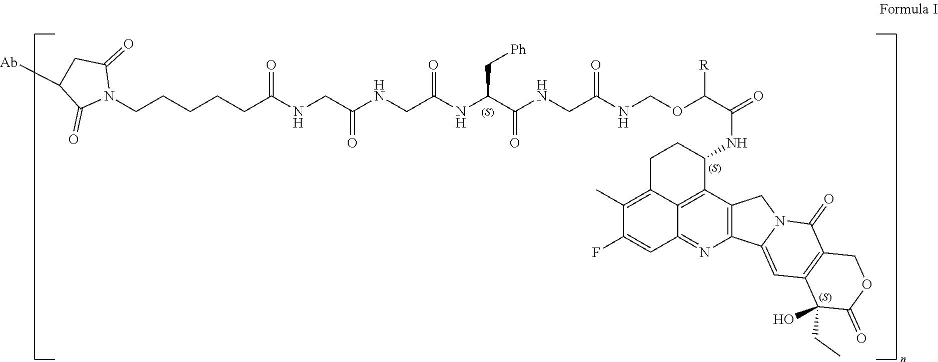
wherein Ab is an antibody targeting HER2, HER3, or EGFR or an antigen-binding fragment thereof, R is selected from C₁₋₆ alkyl, and n is an integer of 1-8 or a decimal of 1-8, preferably, n is 8;
the pharmaceutical composition is an injection, preferably an injection solution or a lyophilized injection;
preferably, the antibody-drug conjugate represented by formula I has a structure represented by formula Ia or formula Ib, or formula I-1, formula Ia-1, or formula Ib-1:

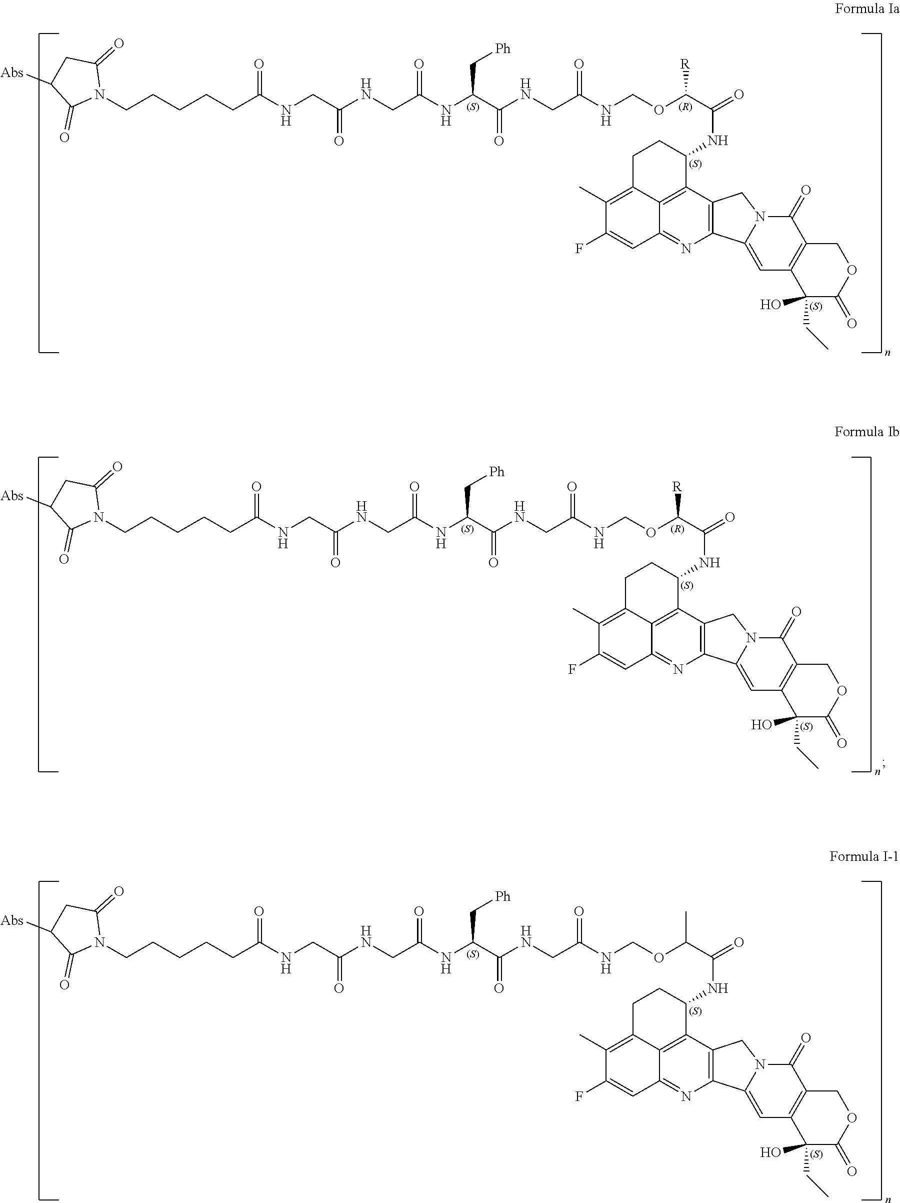

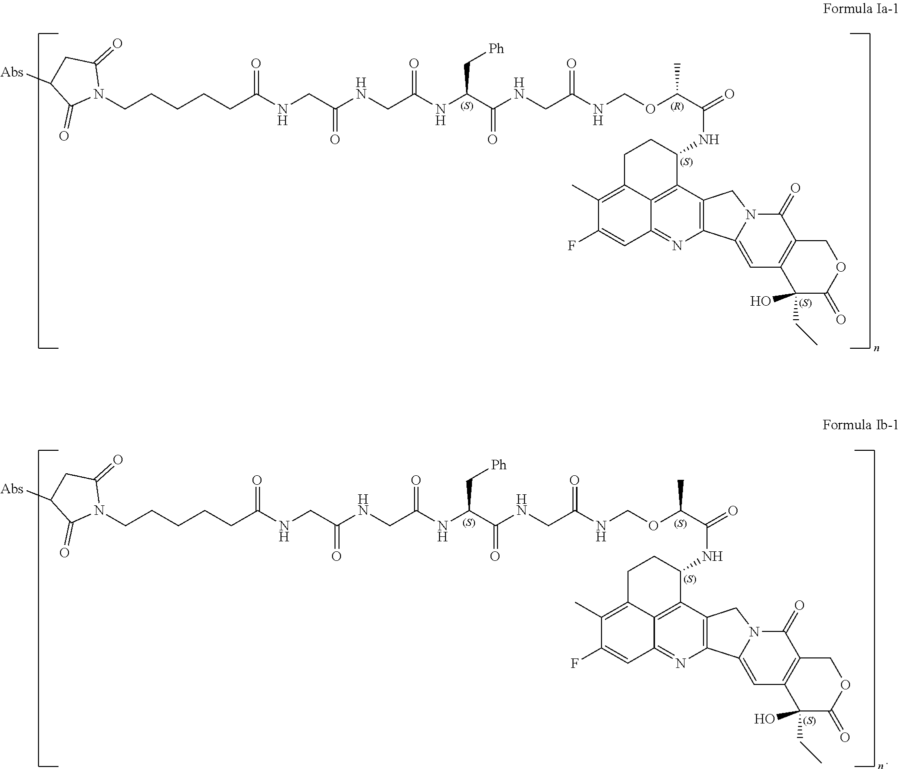

14. The pharmaceutical composition according to claim 13, wherein
in case that the Ab is an antibody targeting HER2 or an antigen-binding fragment thereof, it comprises a variable region of a heavy chain (HV) and a variable region of a light chain (LV), wherein, a sequence of the variable region of the heavy chain comprises a heavy chain complementarity determining region 1 (HCDR1), a heavy chain complementarity determining region 2 (HCDR2) and a heavy chain complementarity determining region 3 (HCDR3), a sequence of the variable region of the light chain comprises a light chain complementarity determining region 1 (LCDR1), a light chain complementarity determining region 2 (LCDR2) and a light chain complementarity determining region 3 (LCDR3), wherein the amino acid sequence of HCDR1 is represented by SEQ ID NO:1, the amino acid sequence of HCDR2 is represented by SEQ ID NO:2, the amino acid sequence of HCDR3 is represented by SEQ ID NO:3, and/or the amino acid sequence of LCDR1 is represented by SEQ ID NO:4, the amino acid sequence of LCDR2 is represented by SEQ ID NO:5, the amino acid sequence of LCDR3 is represented by SEQ ID NO:6;
further preferably, in case that the Ab is an antibody targeting HER2 or an antigen-binding fragment thereof, it comprises a variable region of a heavy chain (HV) and a variable region of a light chain (LV), wherein the amino acid sequence of HV is represented by SEQ ID NO:7, the amino acid sequence of LV is represented by SEQ ID NO:8;
more preferably, in case that the Ab is an antibody targeting HER2 or an antigen-binding fragment thereof, it comprises a heavy chain (HC) and a light chain (LC), wherein the amino acid sequence of HC is represented by SEQ ID NO:9, the amino acid sequence of LC is represented by SEQ ID NO:10;
in case that the Ab is an antibody targeting HER3 or an antigen-binding fragment thereof, it comprises a variable region of a heavy chain (HV) and a variable region of a light chain (LV), wherein, a sequence of the variable region of the heavy chain comprises a heavy chain complementarity determining region 1 (HCDR1), a heavy chain complementarity determining region 2 (HCDR2) and a heavy chain complementarity determining region 3 (HCDR3), a sequence of the variable region of the light chain comprises a light chain complementarity determining region 1 (LCDR1), a light chain complementarity determining region 2 (LCDR2) and a light chain complementarity determining region 3 (LCDR3), wherein the amino acid sequence of HCDR1 is represented by SEQ ID NO:11, the amino acid sequence of HCDR2 is represented by SEQ ID NO:12, the amino acid sequence of HCDR3 is represented by SEQ ID NO:13, and/or the amino acid sequence of LCDR1 is represented by SEQ ID NO:14, the amino acid sequence of LCDR2 is represented by SEQ ID NO:15, the amino acid sequence of LCDR3 is represented by SEQ ID NO:16;
further preferably, in case that the Ab is an antibody targeting HER3 or an antigen-binding fragment thereof, it comprises a variable region of a heavy chain (HV) and a variable region of a light chain (LV), wherein the amino acid sequence of HV is represented by SEQ ID NO:17, the amino acid sequence of LV is represented by SEQ ID NO:18;
more preferably, in case that the Ab is an antibody targeting HER3 or an antigen-binding fragment thereof, it comprises a heavy chain (HC) and a light chain (LC), wherein the amino acid sequence of HC is represented by SEQ ID NO:19, the amino acid sequence of LC is represented by SEQ ID NO:20;
in case that the Ab is an antibody targeting EGFR or an antigen-binding fragment thereof, it comprises a variable region of a heavy chain (HV) and a variable region of a light chain (LV), wherein, a sequence of the variable region of the heavy chain comprises a heavy chain complementarity determining region 1 (HCDR1), a heavy chain complementarity determining region 2 (HCDR2) and a heavy chain complementarity determining region 3 (HCDR3), a sequence of the variable region of the light chain comprises a light chain complementarity determining region 1 (LCDR1), a light chain complementarity determining region 2 (LCDR2) and a light chain complementarity determining region 3 (LCDR3), wherein the amino acid sequence of HCDR1 is represented by SEQ ID NO:21, the amino acid sequence of HCDR2 is represented by SEQ ID NO:22, the amino acid sequence of HCDR3 is represented by SEQ ID NO:23, and/or the amino acid sequence of LCDR1 is represented by SEQ ID NO:24, the amino acid sequence of LCDR2 is represented by SEQ ID NO:25, the amino acid sequence of LCDR3 is represented by SEQ ID NO:26;
in case that the Ab is an antibody targeting EGFR or an antigen-binding fragment thereof, it comprises a variable region of a heavy chain (HV) and a variable region of a light chain (LV), wherein the amino acid sequence of HCDR1 is represented by SEQ ID NO:31 or SEQ ID NO:35 or SEQ ID NO:38, the amino acid sequence of HCDR2 is represented by SEQ ID NO:22, the amino acid sequence of HCDR3 is represented by SEQ ID NO:23 or SEQ ID NO:32, and/or the amino acid sequence of LCDR1 is represented by SEQ ID NO:24, the amino acid sequence of LCDR2 is represented by SEQ ID NO:25, the amino acid sequence of LCDR3 is represented by SEQ ID NO:26;
in case that the Ab is an antibody targeting EGFR or an antigen-binding fragment thereof, it comprises a variable region of a heavy chain (HV) and a variable region of a light chain (LV), wherein the amino acid sequence of HCDR1 is represented by SEQ ID NO:31 or SEQ ID NO:35 or SEQ ID NO:38, the amino acid sequence of HCDR2 is represented by SEQ ID NO:22, the amino acid sequence of HCDR3 is represented by SEQ ID NO:32, and the amino acid sequence of LCDR1 is represented by SEQ ID NO:24, the amino acid sequence of LCDR2 is represented by SEQ ID NO:25, the amino acid sequence of LCDR3 is represented by SEQ ID NO:26;
further preferably, in case that the Ab is an antibody targeting EGFR or an antigen-binding fragment thereof, it comprises a variable region of a heavy chain (HV) and a variable region of a light chain (LV), wherein the amino acid sequence of HV is represented by SEQ ID NO:27, the amino acid sequence of LV is represented by SEQ ID NO:28;
further preferably, in case that the Ab is an antibody targeting EGFR or an antigen-binding fragment thereof, it comprises a variable region of a heavy chain (HV) and a variable region of a light chain (LV), wherein the amino acid sequence of HV is represented by SEQ ID NO:33 or SEQ ID NO:36 or SEQ ID NO:39, the amino acid sequence of LV is represented by SEQ ID NO:28;
more preferably, in case that the Ab is an antibody targeting EGFR or an antigen-binding fragment thereof, it comprises a heavy chain (HC) and a light chain (LC), wherein the amino acid sequence of HC is represented by SEQ ID NO:29, the amino acid sequence of LC is represented by SEQ ID NO:30;
more preferably, in case that the Ab is an antibody targeting EGFR or an antigen-binding fragment thereof, it comprises a heavy chain (HC) and a light chain (LC), wherein the amino acid sequence of HC is represented by SEQ ID NO:34 or SEQ ID NO:37 or SEQ ID NO:40, the amino acid sequence of LC is represented by SEQ ID NO:30.

15. The pharmaceutical composition according to claim 13, wherein the antibody-drug conjugate represented by formula I has the following structure: 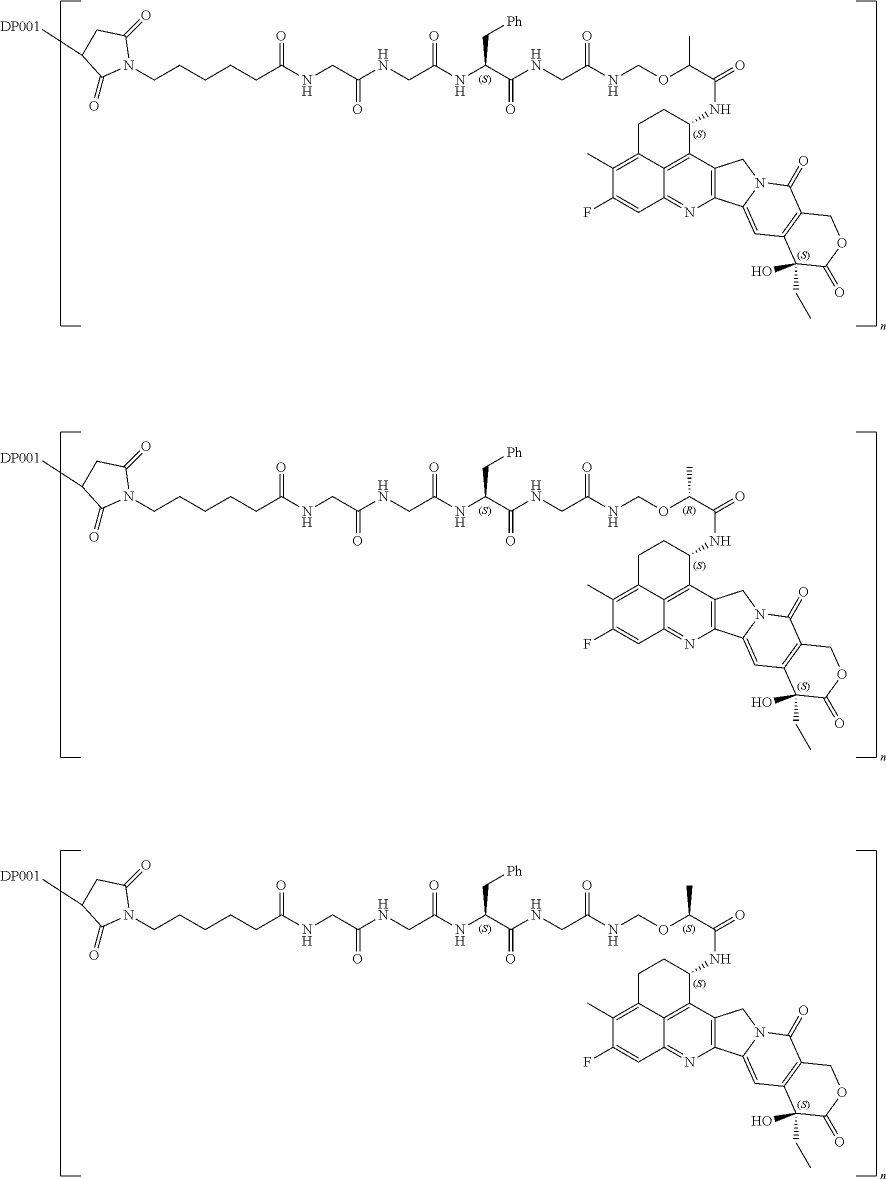 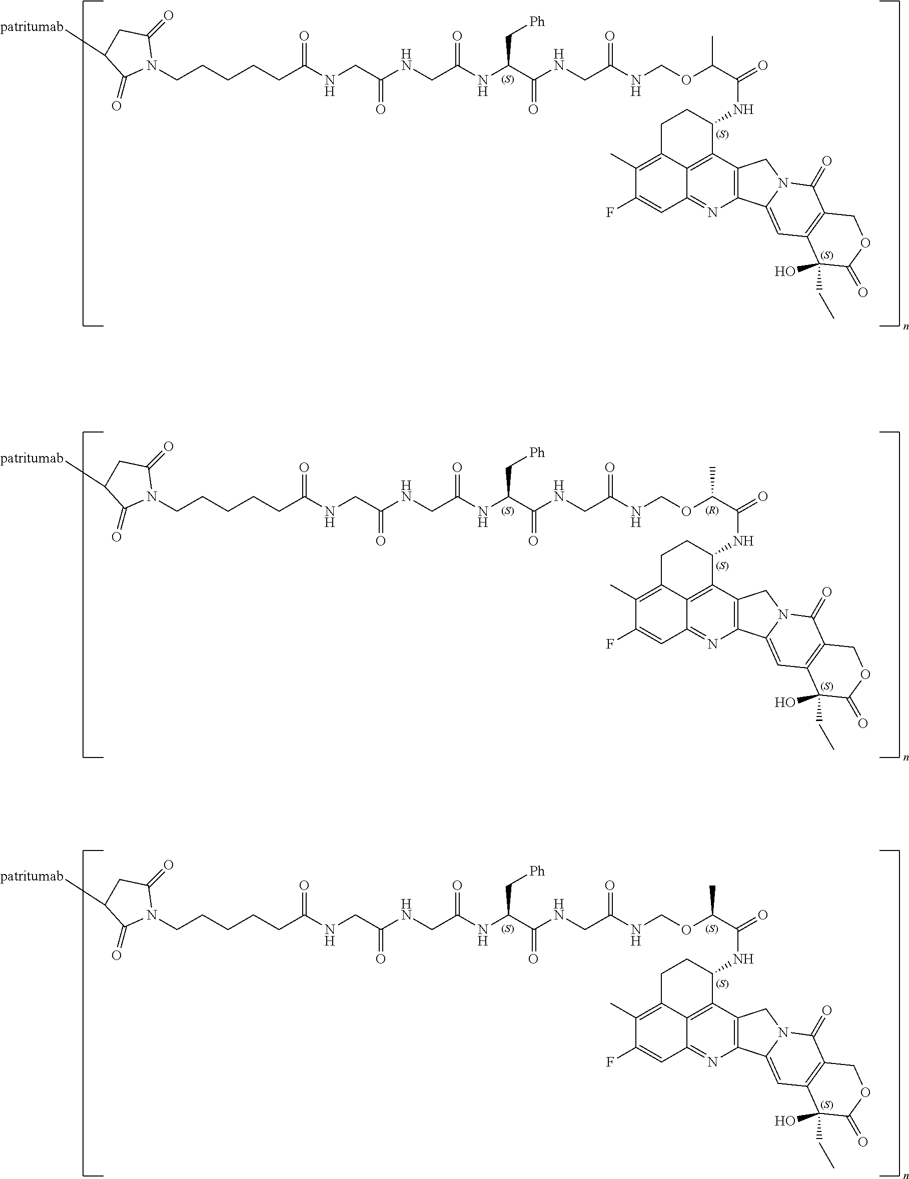 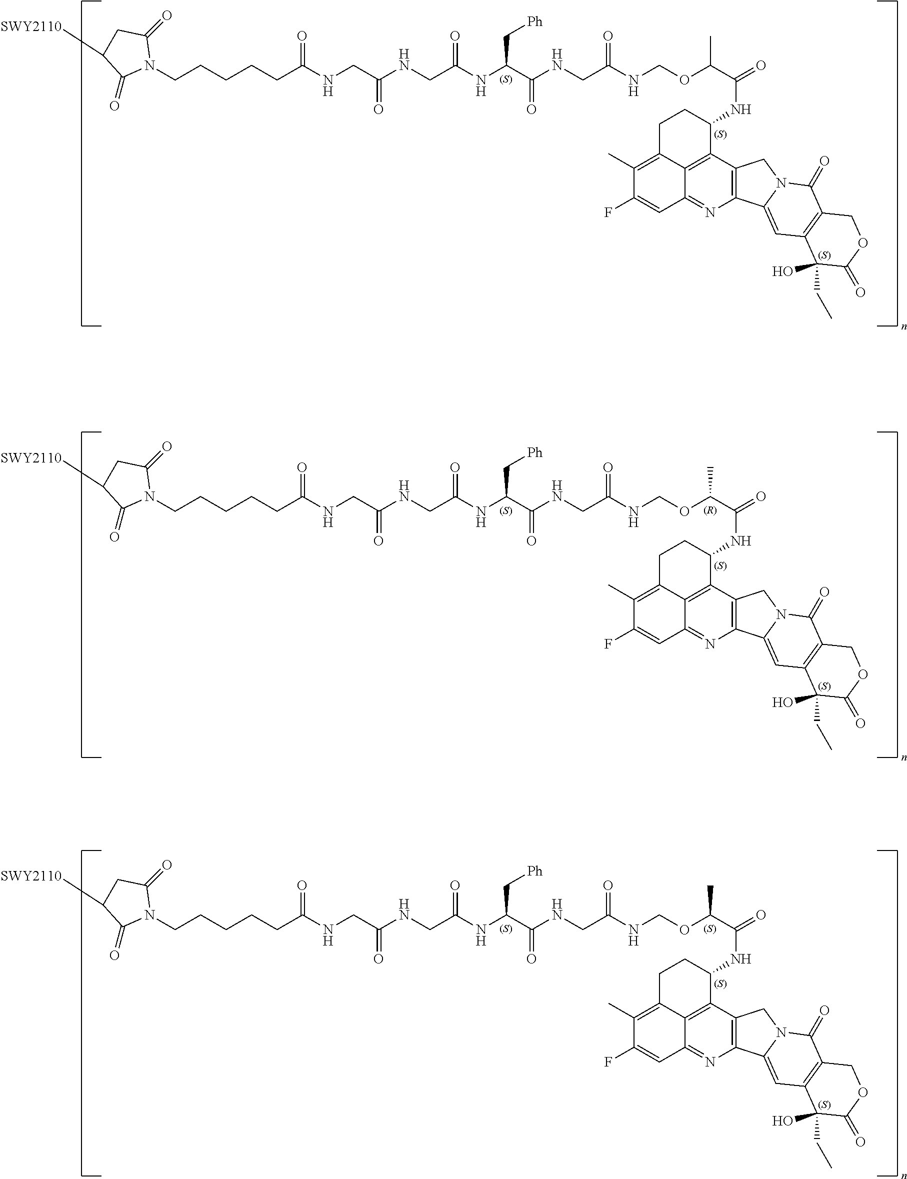 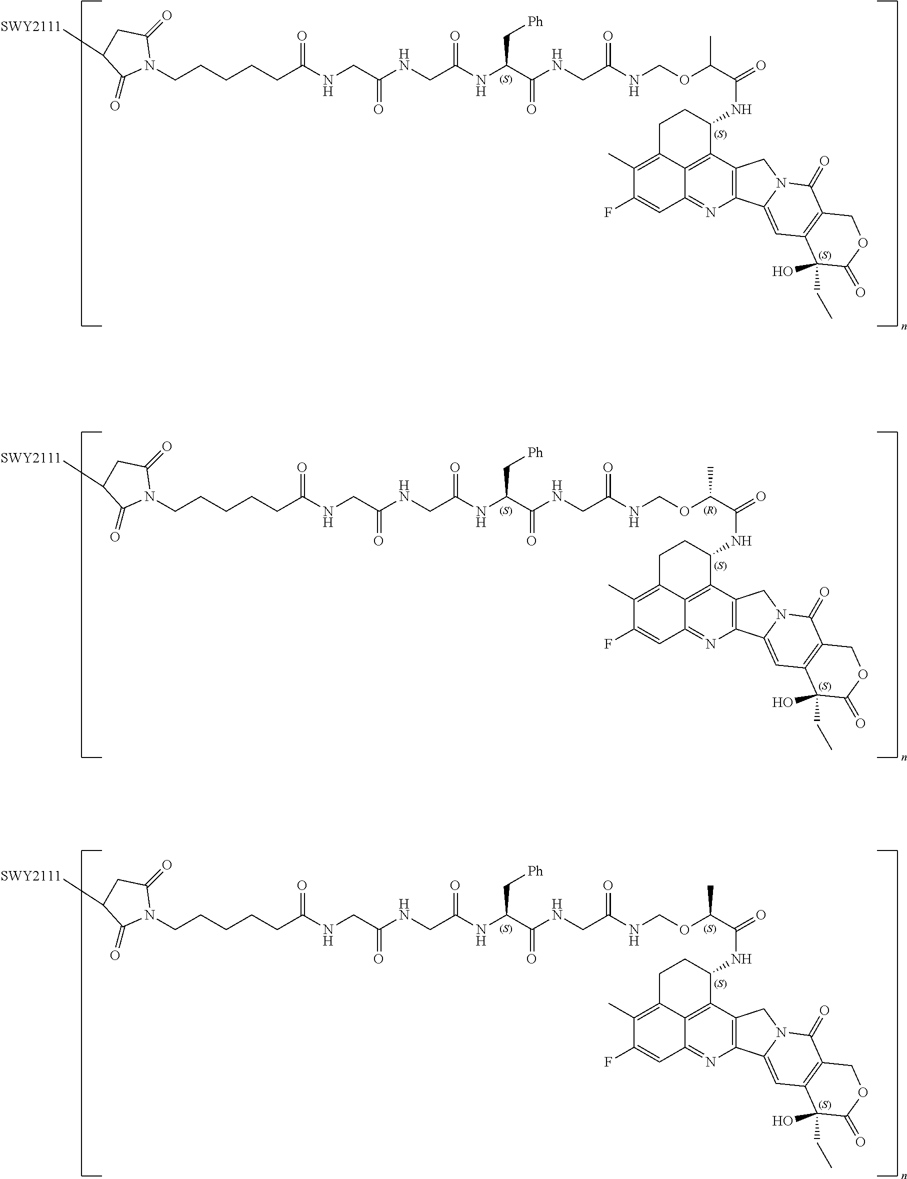 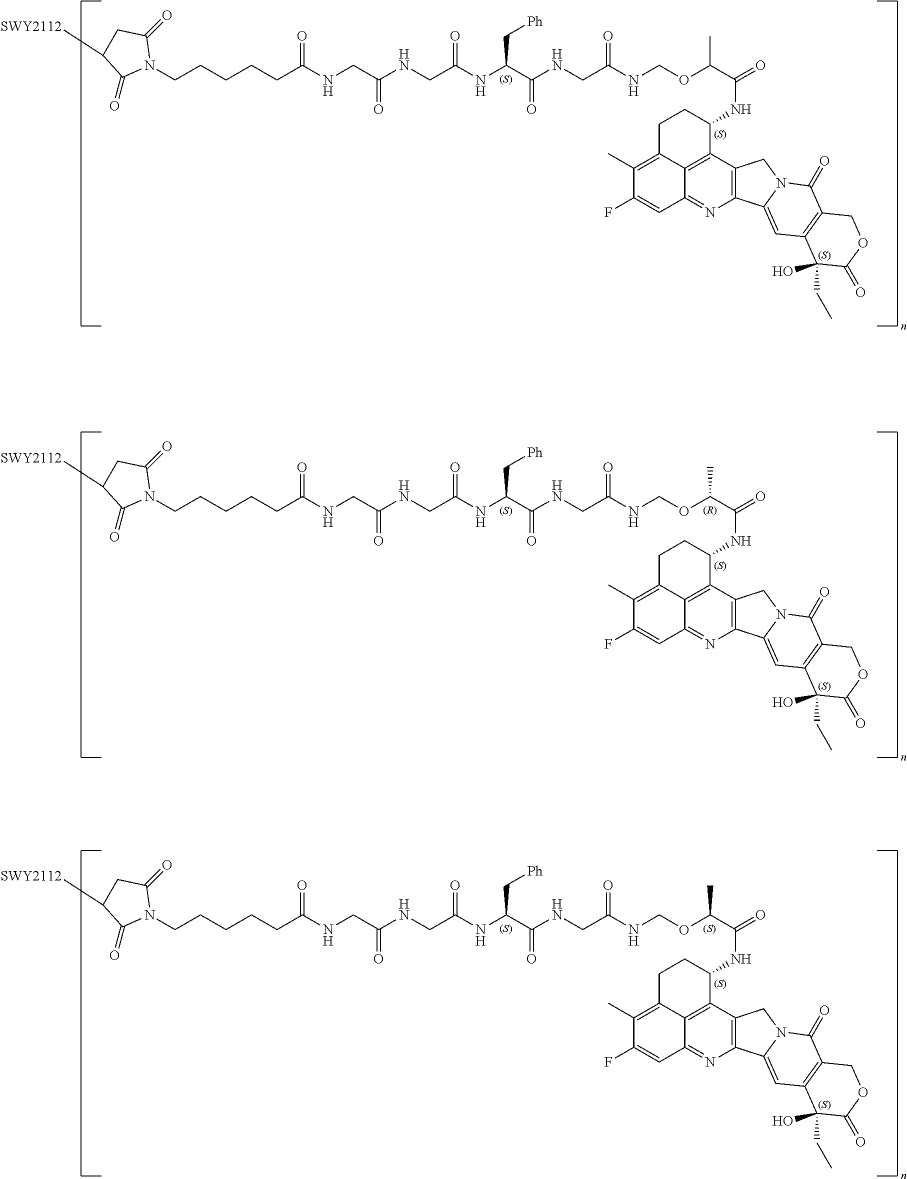 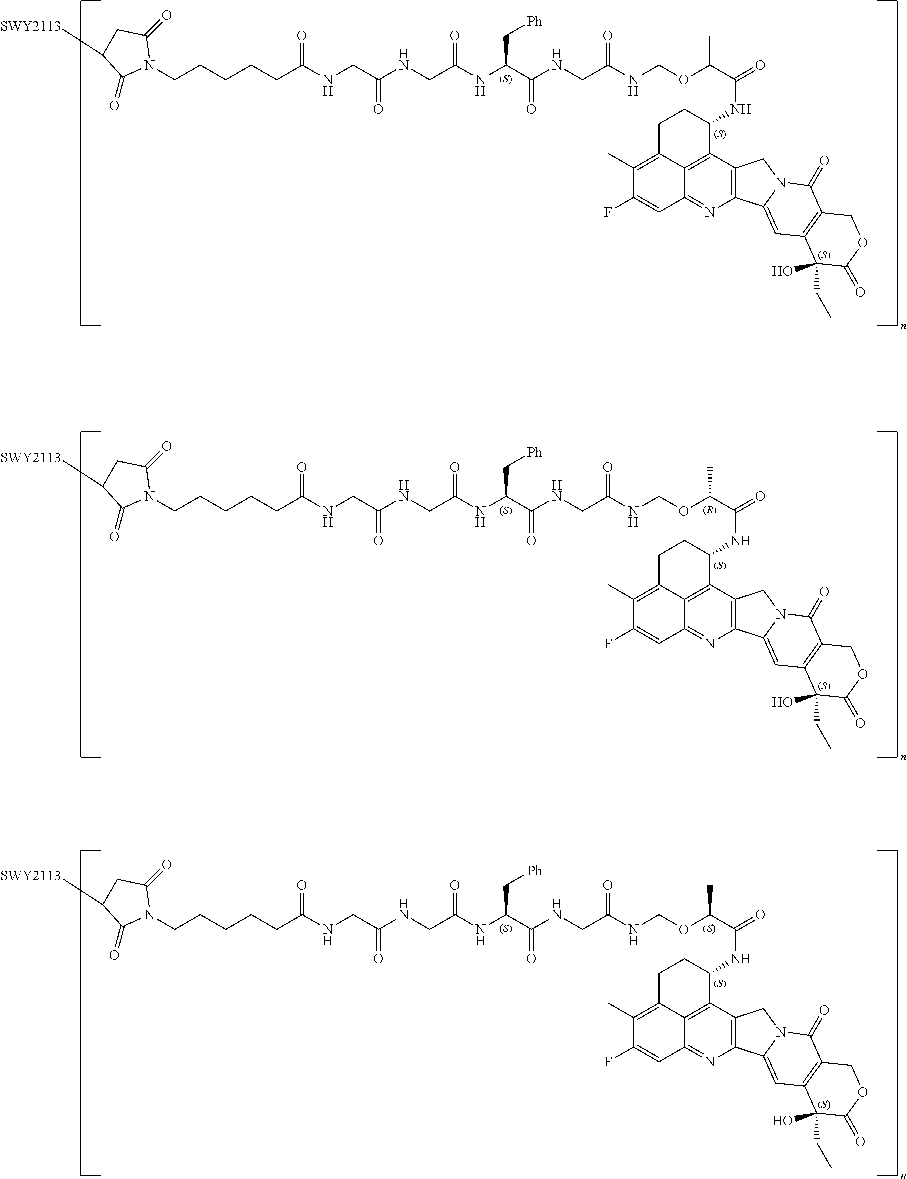
wherein:
DP001 is an antibody having two heavy chains with the amino acid sequence represented by SEQ ID NO:9 and two light chains with the amino acid sequence represented by SEQ ID NO:10;
Patritumab is an antibody having two heavy chains with the amino acid sequence represented by SEQ ID NO:19 and two light chains with the amino acid sequence represented by SEQ ID NO:20;
SWY2110 is an antibody having two heavy chains with the amino acid sequence represented by SEQ ID NO:29 and two light chains with the amino acid sequence represented by SEQ ID NO:30;
SWY2111 is an antibody having two heavy chains with the amino acid sequence represented by SEQ ID NO:34 and two light chains with the amino acid sequence represented by SEQ ID NO:30;
SWY2112 is an antibody having two heavy chains with the amino acid sequence represented by SEQ ID NO:37 and two light chains with the amino acid sequence represented by SEQ ID NO:30;
SWY2113 is an antibody having two heavy chains with the amino acid sequence represented by SEQ ID NO:40 and two light chains with the amino acid sequence represented by SEQ ID NO:30;
n is an integer of 1-8 or a decimal of 1-8, preferably n is 8.

16. The pharmaceutical composition according to claim 13, wherein the weight percentage content of the antibody-drug conjugate represented by formula I is 1-30%, or about 2%, or about 3%, or about 4%, or about 5%, or about 6%, or about 7%, or about 8%, or about 8.5%, or about 9%, or about 9.5%, or about 10%, or about 10.5%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or 5-25%, or 8-20%, or 8-15%, or 8-10%, or 15-25%, based on 100 wt % of the pharmaceutical composition; or
wherein the content of the antibody-drug conjugate represented by formula I is 1-30 mg/ml, or about 1 mg/ml, or about 2 mg/ml, or about 3 mg/ml, or about 4 mg/ml, or about 5 mg/ml, or about 6 mg/ml, or about 7 mg/ml, or about 8 mg/ml, or about 8.5 mg/ml, or about 9 mg/ml, or about 9.5 mg/ml, or about 10 mg/ml, or about 10.5 mg/ml, or about 11 mg/ml, or about 12 mg/ml, or about 13 mg/ml, or about 14 mg/ml, or about 15 mg/ml, or about 16 mg/ml, or about 17 mg/ml, or about 18 mg/ml, or about 19 mg/ml, or about 20 mg/ml, or about 21 mg/ml, or about 22 mg/ml, or about 23 mg/ml, or about 24 mg/ml, or about 25 mg/ml, or about 26 mg/ml, or about 27 mg/ml, or about 28 mg/ml, or about 29 mg/ml, or about 30 mg/ml, or 5-25 mg/ml, or 5-20 mg/ml, or 5-15 mg/ml, or 15-25 mg/ml.

17. The pharmaceutical composition according to claim 13, wherein
said pharmaceutical composition further contains a buffering agent; and/or
said pharmaceutical composition further contains a stabilizer; and/or
said pharmaceutical composition further contains a surfactant.

18. The pharmaceutical composition according to claim 17, wherein
the buffering agent is selected from acetate, succinate, gluconate, histidine salt, oxalate, lactate, phosphate, citrate, tartrate, fumarate, glycylglycine, tris(hydroxymethyl)aminomethane (Tris), 2-(N-morpholino)ethanesulfonic acid (MES), and other organic acid buffering agents, preferably histidine salt, further preferably histidine-histidine hydrochloride; and/or
the stabilizer is selected from saccharides (such as sucrose and trehalose), polyols (such as mannitol and sorbitol), amino acids (such as L-serine, sodium glutamate, alanine, glycine, and sarcosine), preferably saccharides, further preferably sucrose and/or trehalose; and/or
the surfactant is selected from polysorbates, preferably polysorbate 20 and/or polysorbate 80.

19. The pharmaceutical composition according to claim 17, wherein
the weight percentage content of the buffering agent is 1-10%, or about 1%, or about 2%, or about 2.8%, or about 2.9%, or about 3%, or about 3.1%, or about 3.2%, or about 4%, or about 4.5%, or about 4.6%, or about 4.7%, or about 4.8%, or about 4.9%, or about 5%, or about 5.1%, or about 5.2%, or about 5.3%, or about 5.4%, or about 5.5%, or about 6%, or about 6.5%, or about 7%, or about 8%, or about 9%, or about 9.9%, or about 10%, or 1-90.9%, or 2-6%, or 2-5%, or 4-6%, based on 100 wt % of the pharmaceutical composition; or the content of the buffering agent is 1-10 mg/ml, or about 1 mg/ml, or about 2 mg/ml, or about 3 mg/ml, or about 4 mg/ml, or about 4.5 mg/ml, or about 4.6 mg/ml, or about 4.7 mg/ml, or about 4.8 mg/ml, or about 4.9 mg/ml, or about 5 mg/ml, or about 5.1 mg/ml, or about 5.2 mg/ml, or about 5.3 mg/ml, or about 5.4 mg/ml, or about 5.5 mg/ml, or about 6 mg/ml, or about 6.5 mg/ml, or about 7 mg/ml, or about 8 mg/ml, or about 9 mg/ml, or about 10 mg/ml, or 2-6 mg/ml, or 2-4 mg/ml, or 3-6 mg/ml, or 4-6 mg/ml; and/or
the weight percentage content of the stabilizer is 50-90%, or about 50%, or about 60%, or about 70%, or about 75%, or about 80%, or about 81%, or about 82%, or about 83%, or about 84%, or about 85%, or about 86%, or about 87%, or about 88%, or about 89%, or about 90%, or 60-90%, or 70-90%, or 80-90%, based on 100 wt % of the pharmaceutical composition; or the content of the stabilizer is 50-110 mg/ml, or about 50 mg/ml, or about 60 mg/ml, or about 70 mg/ml, or about 75 mg/ml, or about 80 mg/ml, or about 81 mg/ml, or about 82 mg/ml, or about 83 mg/ml, or about 84 mg/ml, or about 85 mg/ml, or about 86 mg/ml, or about 87 mg/ml, or about 88 mg/ml, or about 89 mg/ml, or about 90 mg/ml, or about 91 mg/ml, or about 92 mg/ml, or about 93 mg/ml, or about 94 mg/ml, or about 95 mg/ml, or about 100 mg/ml, or about 105 mg/ml, or about 110 mg/ml, or 70-100 mg/ml, or 80-100 mg/ml, or 75-95 mg/ml; and/or
the weight percentage content of the surfactant is 0.1-1%, or about 0.1%, or about 0.15%, or about 0.16%, or about 0.17%, or about 0.18%, or about 0.19%, or about 0.2%, or about 0.25%, or about 0.26%, or about 0.27%, or about 0.28%, or about 0.29%, or about 0.3%, or about 0.31%, or about 0.32%, or about 0.33%, or about 0.34%, or about 0.35%, or about 0.4%, or about 0.45%, or about 0.5%, or about 0.55%, or about 0.6%, or about 0.7%, or about 0.8%, or about 0.9%, or about 1%, or 0.2-0.4%, or 0.15-0.4%, or 0.15-0.35%, or 0.25-0.35%, based on 100 wt % of the pharmaceutical composition; or the content of the surfactant is 0.1-1 mg/ml, or about 0.1 mg/ml, or about 0.15 mg/ml, or about 0.16 mg/ml, or about 0.17 mg/ml, or about 0.18 mg/ml, or about 0.19 mg/ml, or about 0.2 mg/ml, or about 0.25 mg/ml, or about 0.3 mg/ml, or about 0.35 mg/ml, or about 0.4 mg/ml, or about 0.45 mg/ml, or about 0.5 mg/ml, or about 0.55 mg/ml, or about 0.6 mg/ml, or about 0.7 mg/ml, or about 0.8 mg/ml, or about 0.9 mg/ml, or about 1 mg/ml, or 0.1-0.4 mg/ml, or 0.15-0.25 mg/ml, or 0.2-0.4 mg/ml, or 0.25-0.35 mg/ml.

20. The pharmaceutical composition according to claim 17, wherein the buffering agent is a histidine-histidine hydrochloride buffering agent,
wherein the weight percentage content of histidine is 0.1-1%, or about 0.1%, or about 0.2%, or about 0.25%, or about 0.26%, or about 0.27%, or about 0.28%, or about 0.29%, or about 0.3%, or about 0.31%, or about 0.32%, or about 0.33%, or about 0.34%, or about 0.35%, or about 0.4%, or about 0.45%, or about 0.5%, or about 0.55%, or about 0.6%, or about 0.7%, or about 0.8%, or about 0.9%, or about 1%, or 0.1-0.5%, or 0.2-0.4%, based on 100 wt % of the pharmaceutical composition; or wherein the content of histidine is 0.1-1 mg/ml, or about 0.1 mg/ml, or about 0.2 mg/ml, or about 0.21 mg/ml, or about 0.22 mg/ml, or about 0.23 mg/ml, or about 0.24 mg/ml, or about 0.25 mg/ml, or about 0.26 mg/ml, or about 0.27 mg/ml, or about 0.28 mg/ml, or about 0.29 mg/ml, or about 0.3 mg/ml, or about 0.31 mg/ml, or about 0.32 mg/ml, or about 0.33 mg/ml, or about 0.34 mg/ml, or about 0.35 mg/ml, or about 0.4 mg/ml, or about 0.45 mg/ml, or about 0.5 mg/ml, or about 0.55 mg/ml, or about 0.6 mg/ml, or about 0.7 mg/ml, or about 0.8 mg/ml, or about 0.9 mg/ml, or about 1 mg/ml, or 0.1-0.5 mg/ml, or 0.1-0.3 mg/ml, or 0.2-0.4 mg/ml; and/or
wherein the weight percentage content of histidine hydrochloride is 1-10%, or about 1%, or about 2%, or about 2.5%, or about 2.6%, or about 2.7%, or about 2.8%, or about 2.9%, or about 3%, or about 3.5%, or about 4%, or about 4.5%, or about 4.6%, or about 4.7%, or about 4.8%, or about 4.9%, or about 5%, or about 5.1%, or about 5.2%, or about 5.3%, or about 5.4%, or about 5.5%, or about 6%, or about 6.5%, or about 7%, or about 8%, or about 9%, or about 9.9%, or about 10%, or 1-9.9%, or 2-6%, or 2-4%, or 3-6%, or 3.5-5.5%, or 4-5%, based on 100 wt % of the pharmaceutical composition; or wherein the content of histidine hydrochloride is 1-10 mg/ml, or about 1 mg/ml, or about 2 mg/ml, or about 2.5 mg/ml, or about 2.6 mg/ml, or about 2.7 mg/ml, or about 2.8 mg/ml, or about 2.9 mg/ml, or about 3 mg/ml, or about 3.5 mg/ml, or about 4 mg/ml, or about 4.5 mg/ml, or about 4.6 mg/ml, or about 4.7 mg/ml, or about 4.8 mg/ml, or about 4.9 mg/ml, or about 5 mg/ml, or about 5.1 mg/ml, or about 5.2 mg/ml, or about 5.3 mg/ml, or about 5.4 mg/ml, or about 5.5 mg/ml, or about 6 mg/ml, or about 6.5 mg/ml, or about 7 mg/ml, or about 8 mg/ml, or about 9 mg/ml, or about 10 mg/ml, or 2-6 mg/ml, or 2-4 mg/ml, or 3-6 mg/ml, or 4-5.5 mg/ml, or 4.5-5 mg/ml.

21. The pharmaceutical composition according to claim 13, wherein the pharmaceutical composition has a pH of 4.0-7.5, or about 4.0, or about 4.5, or about 4.6, or about 4.7, or about 4.8, or about 4.9, or about 5.0, or about 5.1, or about 5.2, or about 5.3, or about 5.4, or about 5.5, or about 5.6, or about 5.7, or about 5.8, or about 5.9, or about 6.0, or about 6.5, or about 7.0, or 4.5-7.0, or 4.5-5.5, or 5.0-5.5, or about 5.0.

22. The pharmaceutical composition according to claim 13, wherein
the pharmaceutical composition is a lyophilized injection, and when the lyophilized injection is diluted with water for injection, the diluted pharmaceutical composition contains about 10 mg/ml of the antibody-drug conjugate represented by formula I, about 0.33 mg/ml of histidine, about 4.79 mg/ml of histidine hydrochloride, about 0.3 mg/ml of polysorbate 80, and about 90 mg/ml of trehalose, based on the total volume of the diluted pharmaceutical composition, and the pH of the diluted pharmaceutical composition is about 5.0, preferably, the antibody-drug conjugate represented by formula I is selected from SWY2110-JSSW-001, SWY2111-JSSW-001, and SWY2113-JSSW-001, preferably SWY2110-JSSW-001; or
the pharmaceutical composition is an injection solution, and the injection solution contains about 10 mg/ml of the antibody-drug conjugate represented by formula I, about 0.33 mg/ml of histidine, about 4.79 mg/ml of histidine hydrochloride, about 0.3 mg/ml of polysorbate 80, and about 90 mg/ml of trehalose, and the pH of the injection solution is about 5.0, preferably, the antibody-drug conjugate represented by formula I is selected from SWY2110-JSSW-001, SWY2111-JSSW-001, and SWY2113-JSSW-001, preferably SWY2110-JSSW-001; or
the pharmaceutical composition contains about 10 mg/ml of the antibody-drug conjugate represented by formula I, about 0.33 mg/ml of histidine, about 4.79 mg/ml of histidine hydrochloride, about 0.3 mg/ml of polysorbate 80, about 90 mg/ml trehalose, and the balance of water, and the pH of the pharmaceutical composition is about 5.0, preferably, the antibody-drug conjugate represented by formula I is selected from SWY2110-JSSW-001, SWY2111-JSSW-001, and SWY2113-JSSW-001, preferably SWY2110-JSSW-001; or
the pharmaceutical composition contains or consists of about 10 parts by weight of the antibody-drug conjugate represented by formula I, about 0.33 parts by weight of histidine, about 4.79 parts by weight of histidine hydrochloride, about 0.3 parts by weight of polysorbate 80, and about 90 parts by weight of trehalose, preferably, the antibody-drug conjugate represented by formula I is selected from SWY2110-JSSW-001, SWY2111-JSSW-001, and SWY2113-JSSW-001, preferably SWY2110-JSSW-001; or
the pharmaceutical composition is a lyophilized injection, and when the lyophilized injection is diluted with water for injection, the diluted pharmaceutical composition contains about 20 mg/ml of the antibody-drug conjugate represented by formula I, about 0.21 mg/ml of histidine, about 2.86 mg/ml of histidine hydrochloride, about 0.2 mg/ml of polysorbate 80, and about 80 mg/ml of sucrose, based on the total volume of the diluted pharmaceutical composition, and the pH of the diluted pharmaceutical composition is about 5.0, preferably, the antibody-drug conjugate represented by formula I is patritumab-JSSW-001; or
the pharmaceutical composition is an injection solution, and the injection solution contains about 20 mg/ml of the antibody-drug conjugate represented by formula I, about 0.21 mg/ml of histidine, about 2.86 mg/ml of histidine hydrochloride, about 0.2 mg/ml of polysorbate 80, and about 80 mg/ml of sucrose, and the pH of the injection solution is about 5.0, preferably, the antibody-drug conjugate represented by formula I is patritumab-JSSW-001; or
the pharmaceutical composition contains about 20 mg/ml of the antibody-drug conjugate represented by formula I, about 0.21 mg/ml of histidine, about 2.86 mg/ml of histidine hydrochloride, about 0.2 mg/ml of polysorbate 80, about 80 mg/ml of sucrose, and the balance of water, and the pH of the pharmaceutical composition is about 5.0, preferably, the antibody-drug conjugate represented by formula I is patritumab-JSSW-001; or
the pharmaceutical composition contains or consists of about 20 parts by weight of the antibody-drug conjugate represented by formula I, about 0.21 parts by weight of histidine, about 2.86 parts by weight of histidine hydrochloride, about 0.2 parts by weight of polysorbate 80, and about 80 parts by weight of sucrose, preferably, the antibody-drug conjugate represented by formula I is patritumab-JSSW-001.

23. A method for preparing the pharmaceutical composition according to claim 13, comprising the following steps:
S1) preparing the antibody-drug conjugate represented by formula I;
S2) preparing an ultrafiltration-exchanging buffer;
S3) exchanging the antibody-drug conjugate obtained in S1 into the buffer obtained in S2;
preferably, step S2) comprises weighing a buffering agent and a stabilizer according to the amounts in a formula, adding water for injection to dilute to the target formulation volume, stirring and mixing uniformly to obtain the ultrafiltration-exchanging buffer; step S3) comprises exchanging a certain amount of the antibody-drug conjugate obtained in S1 into the ultrafiltration-exchanging buffer obtained in S2 with an ultrafiltration membrane, diluting the antibody-drug conjugate with water for injection to the target concentration, and then adding the surfactant according to its mass fraction or its volume fraction to obtain a stock solution; optionally, the stock solution is lyophilized, preferably, the lyophilization comprises: (1) pre-freezing, (2) primary drying, and (3) secondary drying, wherein
(1) the conditions for pre-freezing are: at −60 to −30° C. for 18-1800 minutes, e.g. at −45° C. for 180 minutes;
(2) the conditions for the primary drying are: at −25 to 0° C. under 0.02-0.5 mbar (absolute pressure) for 300 minutes to 40000 minutes, e.g. at −5° C. under 0.06 mbar for 3000 minutes, or at −20° C. under 0.20 mbar for 4000 minutes;
(3) the conditions for the secondary drying are: at 15 to 45° C. under 0.1-0.5 mbar (absolute pressure) for 90 minutes to 12000 minutes, e.g. at 30° C. under 0.20 mbar for 1200 minutes, or at 35° C. under 0.25 mbar for 900 minutes.

24. A method of treating a proliferative disease, which comprises administrating the pharmaceutical composition according to claim 13 to a subject in need thereof, preferably, said proliferative disease is preferably a disease associated with abnormal expression of HER2, HER3 or EGFR, including cancer, preferably, the cancer is selected from breast cancer, ovarian cancer, cervical cancer, uterine cancer, prostate cancer, kidney cancer, urethra cancer, bladder cancer, liver cancer, gastric cancer, endometrial cancer, salivary gland cancer, esophageal cancer, melanoma, neuroglioma, neuroblastoma, sarcoma, lung cancer (e.g. small cell lung cancer and non-small cell lung cancer), colon cancer, rectal cancer, colorectal cancer, leukemia (e.g. acute lymphoblastic leukemia, acute myeloid leukemia, acute promyelocytic leukemia, chronic myelocytic leukemia, chronic lymphocytic leukemia), bone cancer, skin cancer, thyroid cancer, pancreatic cancer or lymphoma (e.g. Hodgkin lymphoma, non-Hodgkin lymphoma or relapsed anaplastic large cell lymphoma); optionally, the cancer further comprises triple-negative breast cancer, esophagus cancer, pancreatic cancer, biliary tract cancer, HR-positive breast cancer, lung squamous carcinoma, non-squamous non-small cell lung cancer, head and neck squamous carcinoma (including nasopharyngeal carcinoma), urothelial carcinoma.

25. A method of treating a drug-resistant proliferative disease, which comprises administrating the pharmaceutical composition according to claim 13 to a subject in need thereof, preferably the drug-resistant proliferative disease is a drug-resistant disease associated with abnormal expression of HER2, HER3 or EGFR, including cancer; more preferably, the drug-resistant cancer is a drug-resistant cancer caused by mutation in HER2, HER3 or EGFR genes, the cancer is preferably selected from breast cancer, ovarian cancer, cervical cancer, uterine cancer, prostate cancer, kidney cancer, urethra cancer, bladder cancer, liver cancer, gastric cancer, endometrial cancer, salivary gland cancer, esophageal cancer, melanoma, neuroglioma, neuroblastoma, sarcoma, lung cancer (e.g. small cell lung cancer and non-small cell lung cancer), colon cancer, rectal cancer, colorectal cancer, leukemia (e.g. acute lymphoblastic leukemia, acute myeloid leukemia, acute promyelocytic leukemia, chronic myelocytic leukemia, chronic lymphocytic leukemia), bone cancer, skin cancer, thyroid cancer, pancreatic cancer or lymphoma (e.g. Hodgkin lymphoma, non-Hodgkin lymphoma or relapsed anaplastic large cell lymphoma), optionally, the cancer further comprises triple-negative breast cancer, esophagus cancer, pancreatic cancer, biliary tract cancer, HR-positive breast cancer, lung squamous carcinoma, non-squamous non-small cell lung cancer, head and neck squamous carcinoma (including nasopharyngeal carcinoma), urothelial carcinoma; further preferably the cancer is colon cancer, rectal cancer, lung cancer or pancreatic cancer; wherein, the drug resistance preferably refers to being resistant to receptor tyrosine kinase inhibitors, further preferably resistant to first-generation, second-generation or third-generation EGFR inhibitors, still further preferably resistant to gefitinib, erlotinib, icotinib, afatinib, dacomitinib, osimertinib or almonertinib, especially resistant to gefitinib, afatinib, osimertinib or almonertinib, more preferably resistant to gefitinib or osimertinib.
